(19) United States
(12) Patent Application Publication
(10) Pub. No.: US 2023/0172171 A1
(43) Pub. Date: Jun. 8, 2023

(54) GENETICALLY MODIFIED NON-HUMAN ANIMAL WITH HUMAN OR CHIMERIC CD94 AND/OR NKG2A

(71) Applicant: Biocytogen Pharmaceuticals (Beijing) Co., Ltd., Beijing (CN)

(72) Inventors: Yuelei Shen, Beijing (CN); Yang Bai, Beijing (CN); Chaoshe Guo, Beijing (CN); Meiling Zhang, Beijing (CN); Rui Huang, Beijing (CN); Chengzhang Shang, Beijing (CN)

(21) Appl. No.: 17/922,692

(22) PCT Filed: May 21, 2021
(86) PCT No.: PCT/CN2021/095255

§ 371 Date: Nov. 1, 2022

(30) Foreign Application Priority Data
May 22, 2020 (CN) 202010444099.X

(51) Int. Cl. A01K 67/027; A61K 49/00; C07K 14/705; C12N 15/90

(52) U.S. Cl. CPC A01K 67/0278; A61K 49/0008; C07K 14/7056; C07K 14/70596; C12N 15/907; A01K 2217/15; A01K 2217/072; A01K 2227/105; A01K 2267/03; C12N 2015/8527

(57) ABSTRACT

The present disclosure relates to genetically modified non-human animals that express a human or chimeric (e.g., humanized) CD94 and/or NKG2A, and methods of use thereof.

### CLAIM OF PRIORITY

This application claims the benefit of Chinese Patent Application App. No. 202010444099.X, filed on May 22, 2020. The entire content of the foregoing application is incorporated herein by reference.

### TECHNICAL FIELD

This disclosure relates to genetically modified animal expressing human or chimeric (e.g., humanized) CD94 and/or NKG2A, and methods of use thereof.

### BACKGROUND

The immune system has developed multiple mechanisms to prevent deleterious activation of immune cells. One such mechanism is the intricate balance between positive and negative co-stimulatory signals delivered to immune cells. Targeting the stimulatory or inhibitory pathways for the immune system is considered to be a potential approach for the treatment of various diseases, e.g., cancers and autoimmune diseases.

The traditional drug research and development for these stimulatory or inhibitory receptors typically use in vitro screening approaches. However, these screening approaches cannot provide the body environment (such as tumor microenvironment, stromal cells, extracellular matrix components and immune cell interaction, etc.), resulting in a higher rate of failure in drug development. In addition, in view of the differences between humans and animals, the test results obtained from the use of conventional experimental animals for in vivo pharmacological test may not reflect the real disease state and the interaction at the targeting sites, resulting in that the results in many clinical trials are significantly different from the animal experimental results. Therefore, the development of humanized animal models that are suitable for human antibody screening and evaluation will significantly improve the efficiency of new drug development and reduce the cost for drug research and development.

### SUMMARY

Because the amino acid sequences of human CD94 and NKG2A are significantly different from the corresponding proteins in rodents (e.g., the sequence identity between human and mouse CD94 protein sequences is only about 55% (See FIG. 21), and the sequence identity between human and mouse NKG2A protein sequences is only about 42% (See FIG. 23)), antibodies targeting human CD94 or NKG2A protein usually do not recognize mouse CD94 or NKG2A protein, respectively. Therefore, wild-type mice cannot be used to screen and evaluate the efficacy of drugs targeting the NKG2A and its ligand CD94. In order to make pre-clinical trials more effective and minimize the failure rate of research and development, there is an urgent need in the field to develop a humanized non-human animal model of CD94 and/or NKG2A.

This disclosure is related to an animal model with human CD94 or chimeric CD94. The animal model can express human CD94 or chimeric CD94 (e.g., humanized CD94) protein in its body. It can be used in the studies on the function of CD94 gene, and can be used in the screening and evaluation of anti-human CD94 antibodies. This disclosure is also related to an animal model with human NKG2A or chimeric NKG2A. The animal model can express human NKG2A or chimeric NKG2A (e.g., humanized NKG2A) protein in its body. It can be used in the studies on the function of NKG2A gene, and can be used in the screening and evaluation of anti-human NKG2A antibodies. In some embodiments, the disclosure is related to NKG2A/CD94 double-gene humanized mice.

In addition, the animal models prepared by the methods described herein can be used in drug screening, pharmacodynamics studies, treatments for immune-related diseases, and cancer therapy for human CD94 and/or NKG2A target sites; they can also be used to facilitate the development and design of new drugs, and save time and cost. In summary, this disclosure provides a powerful tool for studying the function of CD94 and/or NKG2A protein and a platform for screening cancer drugs.

In one aspect, the disclosure is related to a genetically-modified, non-human animal whose genome comprises at least one chromosome comprising a sequence encoding a human or chimeric CD94.

In some embodiments, the sequence encoding the human or chimeric CD94 is operably linked to an endogenous regulatory element at the endogenous CD94 gene locus in the at least one chromosome.

In some embodiments, the sequence encoding a human or chimeric CD94 comprises a sequence encoding an amino acid sequence that is at least 70%, 75%, 80%, 85%, 90%, 95%, 99%, or 100% identical to human CD94 (NP_001337991.1 (SEQ ID NO: 2)).

In some embodiments, the sequence encoding a human or chimeric CD94 comprises a sequence encoding an amino acid sequence that is at least 70%, 75%, 80%, 85%, 90%, 95%, 99%, or 100% identical to SEQ ID NO: 8.

In some embodiments, the sequence encoding a human or chimeric CD94 comprises a sequence that is at least 70%, 75%, 80%, 85%, 90%, 95%, 99%, or 100% identical to amino acids of 37-179 of SEQ ID NO: 2.

In some embodiments, the animal is a mammal, e.g., a monkey, a rodent, or a mouse. In some embodiments, the animal is a mouse.

In some embodiments, the animal does not express endogenous CD94 or expresses a decreased level of endogenous CD94.

In some embodiments, the animal has one or more cells expressing human or chimeric CD94.

In some embodiments, the animal has one or more cells expressing human or chimeric CD94, and a human NKG2A can interact with the expressed human or chimeric CD94, forming a heterodimer that can recognize MHC class I molecules. In some embodiments, the animal has one or more cells expressing human or chimeric CD94, and an endogenous NKG2A can interact with the expressed human or chimeric CD94, forming a heterodimer that can recognized MHC class I molecules.

In one aspect, the disclosure is related to a genetically-modified, non-human animal, in some embodiments, the genome of the animal comprises a replacement of a sequence encoding a region of endogenous CD94 with a sequence encoding a corresponding region of human CD94 at an endogenous CD94 gene locus.

In some embodiments, the sequence encoding the corresponding region of human CD94 is operably linked to an endogenous regulatory element at the endogenous CD94 locus, and one or more cells of the animal expresses a chimeric CD94.

In some embodiments, the animal does not express endogenous CD94.

In some embodiments, the replaced sequence encodes all or a portion of the extracellular region of endogenous CD94.

In some embodiments, the animal has one or more cells expressing a chimeric CD94 having a cytoplasmic region, a transmembrane region, and an extracellular region, in some embodiments, the extracellular region comprises a sequence that is at least 50%, 60%, 70%, 80%, 90%, 95%, or 99% identical to the extracellular region of human CD94.

In some embodiments, the extracellular region of the chimeric CD94 has a sequence that has at least 10, 20, 30, 40, 50, 60, 70, 80, 90, 100, 110, 120, 130, or 140 contiguous amino acids that are identical to a contiguous sequence present in the extracellular region of human CD94.

In some embodiments, the sequence encoding a region of endogenous CD94 comprises exon 1, exon 2, exon 3, exon 4, exon 5, and/or exon 6, or a part thereof, of the endogenous CD94 gene.

In some embodiments, the animal is a mouse, and the sequence encoding a region of endogenous CD94 starts within exon 3 and ends within exon 6 of the endogenous mouse CD94 gene.

In some embodiments, the animal is heterozygous with respect to the replacement at the endogenous CD94 gene locus. In some embodiments, the animal is homozygous with respect to the replacement at the endogenous CD94 gene locus.

In one aspect, the disclosure is related to a method for making a genetically-modified, non-human animal, comprising: replacing in at least one cell of the animal, at an endogenous CD94 gene locus, a sequence encoding a region of an endogenous CD94 with a sequence encoding a corresponding region of human CD94.

In some embodiments, the sequence encoding the corresponding region of human CD94 comprises exon 2, exon 3, exon 4, exon 5, exon 6, and/or exon 7, or a part thereof, of a human CD94 gene.

In some embodiments, the sequence encoding the corresponding region of human CD94 starts within exon 4 and ends within exon 7 of a human CD94 gene.

In some embodiments, the sequence encoding the corresponding region of human CD94 encodes amino acids 37-179 of SEQ ID NO: 2.

In some embodiments, the region of an endogenous CD94 is located within the extracellular region.

In some embodiments, the sequence encoding a region of endogenous CD94 comprises exon 1, exon 2, exon 3, exon 4, exon 5, and/or exon 6, or a part thereof, of the endogenous CD94 gene.

In some embodiments, the animal is a mouse, and the sequence encoding a region of an endogenous CD94 starts within exon 3 and ends within exon 6 of the endogenous mouse CD94 gene.

In one aspect, the disclosure is related to a non-human animal comprising at least one cell comprising a nucleotide sequence encoding a chimeric CD94 polypeptide, in some embodiments, the chimeric CD94 polypeptide comprises at least 50 contiguous amino acid residues that are identical to the corresponding contiguous amino acid sequence of a human CD94, in some embodiments, the animal expresses the chimeric CD94.

In some embodiments, the chimeric CD94 polypeptide has at least 50, at least 60, at least 70, at least 80, at least 90, at least 100, at least 110, at least 120, at least 130, or at least 140 contiguous amino acid residues that are identical to the corresponding contiguous amino acid sequence of a human CD94 extracellular region.

In some embodiments, the chimeric CD94 polypeptide comprises a sequence that is at least 90%, 95%, or 99% identical to amino acids 37-179 of SEQ ID NO: 2.

In some embodiments, the nucleotide sequence is operably linked to an endogenous CD94 regulatory element of the animal.

In some embodiments, the chimeric CD94 polypeptide comprises an endogenous CD94 cytoplasmic region and/or an endogenous CD94 transmembrane region.

In some embodiments, the nucleotide sequence is integrated to an endogenous CD94 gene locus of the animal.

In some embodiments, the chimeric CD94 has at least one mouse CD94 activity and/or at least one human CD94 activity.

In one aspect, the disclosure is related to a method of making a genetically-modified non-human animal cell that expresses a chimeric CD94, the method comprising: replacing at an endogenous CD94 gene locus, a nucleotide sequence encoding a region of endogenous CD94 with a nucleotide sequence encoding a corresponding region of human CD94, thereby generating a genetically-modified non-human animal cell that includes a nucleotide sequence that encodes the chimeric CD94, in some embodiments, the non-human animal cell expresses the chimeric CD94.

In some embodiments, the animal is a mammal, e.g., a monkey, a rodent, or a mouse.

In some embodiments, the chimeric CD94 comprises: a cytoplasmic region and/or a transmembrane region of endogenous CD94; and an extracellular region of human CD94.

In some embodiments, the nucleotide sequence encoding the chimeric CD94 is operably linked to an endogenous CD94 regulatory region, e.g., promoter.

In some embodiments, the animal further comprises a sequence encoding an additional human or chimeric protein. In some embodiments, the additional human or chimeric protein is Killer Cell Lectin Like Receptor C1 (NKG2A), programmed cell death protein 1 (PD-1), B7 Homolog 3 (B7-H3), V-set domain-containing T-cell activation inhibitor 1 (B7-H4), Interleukin-2 (IL-2), Interleukin-23 subunit alpha (IL23A), C-C Motif Chemokine Receptor 2 (CCR2), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α (SIRPα) or TNF Receptor Superfamily Member 4 (OX40).

In some embodiments, the additional human or chimeric protein is NKG2A, and the animal expresses the human or chimeric NKG2A.

In some embodiments, the animal further comprises a sequence encoding human or chimeric PD-1, and the animal expresses the human or chimeric PD-1.

In one aspect, the disclosure is related to a genetically-modified, non-human animal whose genome comprises at least one chromosome comprising a sequence encoding a human or chimeric Killer cell lectin-like receptor subfamily C, member 1 (NKG2A).

In some embodiments, the sequence encoding the human or chimeric NKG2A is operably linked to an endogenous regulatory element at the endogenous NKG2A gene locus in the at least one chromosome.

In some embodiments, the sequence encoding a human or chimeric NKG2A comprises a sequence encoding an amino acid sequence that is at least 70%, 75%, 80%, 85%, 90%, 95%, 99%, or 100% identical to human NKG2A (NP_998823.1 (SEQ ID NO: 29)).

In some embodiments, the sequence encoding a human or chimeric NKG2A comprises a sequence encoding an amino acid sequence that is at least 70%, 75%, 80%, 85%, 90%, 95%, 99%, or 100% identical to SEQ ID NO: 35.

In some embodiments, the sequence encoding a human or chimeric NKG2A comprises a sequence that is at least 70%, 75%, 80%, 85%, 90%, 95%, 99%, or 100% identical to amino acids of 94-233 of SEQ ID NO: 29.

In some embodiments, the animal is a mammal, e.g., a monkey, a rodent, or a mouse. In some embodiments, the animal is a mouse.

In some embodiments, the animal does not express endogenous NKG2A or expresses a decreased level of endogenous NKG2A.

In some embodiments, the animal has one or more cells expressing human or chimeric NKG2A.

In some embodiments, the animal has one or more cells expressing human or chimeric NKG2A, and a human CD94 can interact with the expressed human or chimeric NKG2A, forming a heterodimer that can recognize MHC class I molecules. In some embodiments, the animal has one or more cells expressing human or chimeric NKG2A, and an endogenous CD94 can interact with the expressed human or chimeric NKG2A, forming a heterodimer that can recognized MHC class I molecules.

In one aspect, the disclosure is related to a genetically-modified, non-human animal, in some embodiments, the genome of the animal comprises a replacement of a sequence encoding a region of endogenous NKG2A with a sequence encoding a corresponding region of human NKG2A at an endogenous NKG2A gene locus.

In some embodiments, the sequence encoding the corresponding region of human NKG2A is operably linked to an endogenous regulatory element at the endogenous NKG2A locus, and one or more cells of the animal expresses a chimeric NKG2A.

In some embodiments, the animal does not express endogenous NKG2A.

In some embodiments, the replaced sequence encodes all or a portion of the extracellular region of endogenous NKG2A.

In some embodiments, the animal has one or more cells expressing a chimeric NKG2A having a cytoplasmic region, a transmembrane region, and an extracellular region, in some embodiments, the extracellular region comprises a sequence that is at least 50%, 60%, 70%, 80%, 90%, 95%, or 99% identical to the extracellular region of human NKG2A.

In some embodiments, the extracellular region of the chimeric NKG2A has a sequence that has at least 10, 20, 30, 40, 50, 60, 70, 80, 90, 100, 110, 120, 130, or 140 contiguous amino acids that are identical to a contiguous sequence present in the extracellular region of human NKG2A.

In some embodiments, the sequence encoding a region of endogenous NKG2A comprises exon 1, exon 2, exon 3, exon 4, exon 5, and/or exon 6, or a part thereof, of the endogenous NKG2A gene.

In some embodiments, the animal is a mouse, and the sequence encoding a region of endogenous NKG2A starts within exon 2 and ends within exon 6 of the endogenous mouse NKG2A gene.

In some embodiments, the animal is heterozygous with respect to the replacement at the endogenous NKG2A gene locus. In some embodiments, the animal is homozygous with respect to the replacement at the endogenous NKG2A gene locus.

In one aspect, the disclosure is related to a method for making a genetically-modified, non-human animal, comprising: replacing in at least one cell of the animal, at an endogenous NKG2A gene locus, a sequence encoding a region of an endogenous NKG2A with a sequence encoding a corresponding region of human NKG2A.

In some embodiments, the sequence encoding the corresponding region of human NKG2A comprises exon 3, exon 4, exon 5, exon 6, exon 7, and/or exon 8, or a part thereof, of a human NKG2A gene.

In some embodiments, the sequence encoding the corresponding region of human NKG2A starts within exon 4 and ends within exon 8 of a human NKG2A gene.

In some embodiments, the sequence encoding the corresponding region of human NKG2A encodes amino acids 94-233 of SEQ ID NO: 29.

In some embodiments, the region of an endogenous NKG2A is located within the extracellular region.

In some embodiments, the sequence encoding a region of endogenous NKG2A comprises exon 1, exon 2, exon 3, exon 4, exon 5, and/or exon 6, or a part thereof, of the endogenous NKG2A gene.

In some embodiments, the animal is a mouse, and the sequence encoding a region of an endogenous NKG2A starts within exon 2 and ends within exon 6 of the endogenous mouse NKG2A gene.

In one aspect, the disclosure is related to a non-human animal comprising at least one cell comprising a nucleotide sequence encoding a chimeric NKG2A polypeptide, in some embodiments, the chimeric NKG2A polypeptide comprises at least 50 contiguous amino acid residues that are identical to the corresponding contiguous amino acid sequence of a human NKG2A, in some embodiments, the animal expresses the chimeric NKG2A.

In some embodiments, the chimeric NKG2A polypeptide has at least 50, at least 60, at least 70, at least 80, at least 90, at least 100, at least 110, at least 120, at least 130, or at least 140 contiguous amino acid residues that are identical to the corresponding contiguous amino acid sequence of a human NKG2A extracellular region.

In some embodiments, the chimeric NKG2A polypeptide comprises a sequence that is at least 90%, 95%, or 99% identical to amino acids 94-233 of SEQ ID NO: 29.

In some embodiments, the nucleotide sequence is operably linked to an endogenous NKG2A regulatory element of the animal.

In some embodiments, the chimeric NKG2A polypeptide comprises an endogenous NKG2A cytoplasmic region and/or an endogenous NKG2A transmembrane region.

In some embodiments, the nucleotide sequence is integrated to an endogenous NKG2A gene locus of the animal.

In some embodiments, the chimeric NKG2A has at least one mouse NKG2A activity and/or at least one human NKG2A activity.

In one aspect, the disclosure is related to a method of making a genetically-modified non-human animal cell that expresses a chimeric NKG2A, the method comprising: replacing at an endogenous NKG2A gene locus, a nucleotide sequence encoding a region of endogenous NKG2A with a nucleotide sequence encoding a corresponding region of human NKG2A, thereby generating a genetically-modified non-human animal cell that includes a nucleotide sequence that encodes the chimeric NKG2A, in some embodiments, the non-human animal cell expresses the chimeric NKG2A.

In some embodiments, the animal is a mammal, e.g., a monkey, a rodent, or a mouse.

In some embodiments, the chimeric NKG2A comprises: a cytoplasmic region and/or a transmembrane region of endogenous NKG2A; and an extracellular region of human NKG2A.

In some embodiments, the nucleotide sequence encoding the chimeric NKG2A is operably linked to an endogenous NKG2A regulatory region, e.g., promoter.

In some embodiments, the animal further comprises a sequence encoding an additional human or chimeric protein. In some embodiments, the additional human or chimeric protein is Cluster of Differentiation 94 (CD94), programmed cell death protein 1 (PD-1), B7 Homolog 3 (B7-H3), V-set domain-containing T-cell activation inhibitor 1 (B7-H4), Interleukin-2 (IL-2), Interleukin-23 subunit alpha (IL23A), C-C Motif Chemokine Receptor 2 (CCR2), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α (SIRPα) or TNF Receptor Superfamily Member 4 (OX40).

In some embodiments, the additional human or chimeric protein is CD94, and the animal expresses the human or chimeric CD94.

In some embodiments, the animal further comprises a sequence encoding human or chimeric PD-1, and the animal expresses the human or chimeric PD-1.

In one aspect, the disclosure is related to a method of determining effectiveness of a therapeutic agent recognizing a NKG2A/CD94 receptor for treating cancer, comprising: administering the therapeutic agent to the animal as described herein, in some embodiments, the animal has a cancer; and determining the inhibitory effects of the therapeutic agent to the cancer.

In some embodiments, the therapeutic agent is an anti-CD94 antibody or anti-NKG2A antibody.

In some embodiments, the cancer comprises one or more cells that express MHC class I molecules (e.g., human HLA-E or mouse Qa-1).

In some embodiments, the cancer comprises one or more cancer cells that are injected into the animal.

In some embodiments, determining the inhibitory effects of the therapeutic agent to the cancer involves measuring the tumor volume in the animal.

In some embodiments, the cancer is gynecologic cancer, ovarian cancer, uterine cancer, vaginal cancer, cervical cancer, vulvar cancer, head and neck cancer, non-small cell lung cancer (NSCLC), hematological cancer, solid tumor, breast cancer, chronic lymphocytic leukemia, squamous cell carcinoma of the oral cavity, colorectal cancer, liver cancer, glioblastoma, Hodgkin lymphoma, esophagus cancer, gastric cancer, pancreas cancer, renal cancer, lung cancer, or melanoma.

In one aspect, the disclosure is related to a method of determining effectiveness of an therapeutic agent recognizing a NKG2A/CD94 receptor and an additional therapeutic agent for the treating cancer, comprising administering the therapeutic agent and the additional therapeutic agent to the animal as described herein, in some embodiments, the animal has a cancer; and determining the inhibitory effects on the cancer.

In some embodiments, the animal further comprises a sequence encoding a human or chimeric programmed cell death protein 1 (PD-1).

In some embodiments, the animal further comprises a sequence encoding a human or chimeric programmed death-ligand 1 (PD-L1).

In some embodiments, the additional therapeutic agent is an anti-PD-1 antibody, an anti-PD-L1 antibody, or an anti-EGFR (epidermal growth factor receptor) antibody..

In some embodiments, the cancer comprises one or more cancer cells that express PD-L1, or PD-L2.

In some embodiments, the cancer is caused by injection of one or more cancer cells into the animal.

In some embodiments, determining the inhibitory effects of the treatment involves measuring the tumor volume in the animal.

In some embodiments, the animal has gynecologic cancer, ovarian cancer, uterine cancer, vaginal cancer, cervical cancer, vulvar cancer, head and neck cancer, non-small cell lung cancer (NSCLC), hematological cancer, solid tumor, breast cancer, chronic lymphocytic leukemia, squamous cell carcinoma of the oral cavity, colorectal cancer, liver cancer, glioblastoma, Hodgkin lymphoma, esophagus cancer, gastric cancer, pancreas cancer, renal cancer, lung cancer, or melanoma.

In one aspect, the disclosure is related to a method of determining effectiveness of a therapeutic agent recognizing a NKG2A/CD94 receptor for treating an immune disorder, comprising: administering the therapeutic agent to the animal as described herein, in some embodiments, the animal has the immune disorder; and determining the inhibitory effects of the therapeutic agent to the immune disorder.

In some embodiments, the therapeutic agent is an anti-CD94 antibody or anti-NKG2A antibody.

In some embodiments, the cancer comprises one or more cells that express MHC class I molecules (e.g., human HLA-E or mouse Qa-1).

In some embodiments, the immune disorder is rheumatoid arthritis.

In one aspect, the disclosure is related to a protein comprising an amino acid sequence, in some embodiments, the amino acid sequence is one of the following:
(a) an amino acid sequence set forth in SEQ ID NO: 1, 2, 8, 28, 29, or 35;
(b) an amino acid sequence that is at least 90% identical to SEQ ID NO: 1, 2, 8, 28, 29, or 35;
(c) an amino acid sequence that is at least 91%, 92%, 93%, 94%, 95%, 96%, 97%, 98%, or 99% identical to SEQ ID NO: 1, 2, 8, 28, 29, or 35;
(d) an amino acid sequence that is different from the amino acid sequence set forth in SEQ ID NO: 1, 2, 8, 28, 29, or 35 by no more than 10, 9, 8, 7, 6, 5, 4, 3, 2 or 1 amino acid; and
(e) an amino acid sequence that comprises a substitution, a deletion and /or insertion of one, two, three, four, five or more amino acids to the amino acid sequence set forth in SEQ ID NO: 1, 2, 8, 28, 29, or 35.

In one aspect, the disclosure is related to a nucleic acid comprising a nucleotide sequence, in some embodiments, the nucleotide sequence is one of the following:
(a) a sequence that encodes the protein as described herein;
(b) SEQ ID NO: 3, 4, 5, 6, 7, 9, 10, 30, 31, 32, 33, 34, 36, or 37;
(c) a sequence that is at least 90% identical to SEQ ID NO: 3, 4, 5, 6, 7, 9, 10, 30, 31, 32, 33, 34, 36, or 37; and
(d) a sequence that is at least 91%, 92%, 93%, 94%, 95%, 96%, 97%, 98%, or 99% identical to SEQ ID NO: 3, 4, 5, 6, 7, 9, 10, 30, 31, 32, 33, 34, 36, or 37.

In one aspect, the disclosure is related to a cell comprising the protein and/or the nucleic acid as described herein. In one aspect, the disclosure is related to an animal comprising the protein and/or the nucleic acid as described herein.

In another aspect, the disclosure also provides a genetically-modified, non-human animal whose genome comprise a disruption in the animal’s endogenous NKG2A gene, wherein the disruption of the endogenous NKG2A gene comprises deletion of exon 1, exon 2, exon 3, exon 4, exon 5, exon 6, and/or exon 7, or part thereof of the endogenous NKG2A gene.

In some embodiments, the disruption of the endogenous NKG2A gene comprises deletion of one or more exons or part of exons selected from the group consisting of exon 2, exon 3, exon 4, exon 5, and/or exon 6 of the endogenous NKG2A gene.

In some embodiments, the disruption of the endogenous NKG2A gene comprises deletion of a portion of exon 2, exons 3-5, and a portion of exon 6 of the endogenous NKG2A gene. In some embodiments, the disruption of the endogenous NKG2A gene also comprises deletion of intron 2, intron 3, intron 4, and/or intron 5 of the endogenous NKG2A gene. In some embodiments, the disruption of the endogenous NKG2A gene comprises a nucleotide sequence within endogenous NKG2A gene exon 2 encoding the last amino acid. In some embodiments, the disruption of the endogenous NKG2A gene comprises a nucleotide sequence encoding the extracellular region of endogenous NKG2A.

In some embodiments, the disruption of the endogenous NKG2A gene further comprises deletion of one or more introns or part of introns selected from the group consisting of intron 2, intron 3, intron 4, and intron 5 of the endogenous NKG2A gene.

In some embodiments, the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, 300, 350, 400, 450, 500, 550, 600, 1000, 1500, 2000, 2500, 3000, 3400, or more nucleotides.

In some embodiments, the disruption of the endogenous gene comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, 300, 350, 400, 450, 500, 550, 600, or more nucleotides of exon 1, exon 2, exon 3, exon 4, exon 5, exon 6, and/or exon 7 (e.g., deletion of a nucleotide sequence starting from the first few nucleotide (e.g., 1 ,2 ,3, 4, or 5) of the extracellular region-encoding sequence to the last nucleotide of the extracellular region-encoding sequence).

In one aspect, the disclosure is related to a cell comprising the protein as described herein and/or the nucleic acid as described herein. In one aspect, the disclosure is related to an animal comprising the protein as described herein and/or the nucleic acid as described herein.

The disclosure also relates to non-human mammal generated through the methods as described herein. In some embodiments, the genome thereof contains human gene(s). In some embodiments, the non-human mammal is a rodent. In some embodiments, the non-human mammal is a mouse. In some embodiments, the non-human mammal expresses a protein encoded by a humanized CD94 or NKG2A gene.

The disclosure also relates to an offspring of the non-human mammal.

In another aspect, the disclosure relates to a tumor bearing non-human mammal model, characterized in that the non-human mammal model is obtained through the methods as described herein. In some embodiments, the non-human mammal is a rodent. In some embodiments, the non-human mammal is a mouse.

The disclosure also relates to a cell (e.g., stem cell or embryonic stem cell) or cell line, or a primary cell culture thereof derived from the non-human mammal or an offspring thereof, or the tumor bearing non-human mammal.

The disclosure further relates to the tissue, organ or a culture thereof derived from the non-human mammal or an offspring thereof, or the tumor bearing non-human mammal.

In another aspect, the disclosure relates to a tumor tissue derived from the non-human mammal or an offspring thereof when it bears a tumor, or the tumor bearing non-human mammal.

The disclosure further relates to a CD94 or NKG2A genomic DNA sequence of a humanized mouse, a DNA sequence obtained by a reverse transcription of the mRNA obtained by transcription thereof is consistent with or complementary to the DNA sequence; a construct expressing the amino acid sequence thereof; a cell comprising the construct thereof; a tissue comprising the cell thereof.

The disclosure further relates to the use of the non-human mammal or an offspring thereof, or the tumor bearing non-human mammal, the animal model generated through the method as described herein in the development of a product related to an immunization processes of human cells, the manufacture of a human antibody, or the model system for a research in pharmacology, immunology, microbiology and medicine.

The disclosure also relates to the use of the non-human mammal or an offspring thereof, or the tumor bearing non-human mammal, the animal model generated through the method as described herein in the production and utilization of an animal experimental disease model of an immunization processes involving human cells, the study on a pathogen, or the development of a new diagnostic strategy and /or a therapeutic strategy.

The disclosure further relates to the use of the non-human mammal or an offspring thereof, or the tumor bearing non-human mammal, the animal model generated through the methods as described herein, in the screening, verifying, evaluating or studying the CD94 or NKG2A gene function, human CD94 or NKG2A antibodies, the drugs or efficacies for human CD94 or NKG2A targeting sites, and the drugs for immune-related diseases and antitumor drugs.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a schematic diagram showing mouse CD94 gene locus.

FIG. 1B is a schematic diagram showing human CD94 gene locus.

FIG. 2 is a schematic diagram showing humanized CD94 gene locus. A mouse CD94 gene sequence starting from exon 3 and ending within exon 6 was replaced with a human CD94 gene sequence starting from exon 4 and ending within exon 7.

FIG. 3 is a schematic diagram showing a CD94 gene targeting strategy. The targeting vector includes a 5′ homologous arm, a 3′ homologous arm, and a knock-in (KI) fragment (including a human CD94 gene DNA fragment and Neo cassette). ADTA sequence is included at the downstream of the 3′ homologous arm.

FIG. 4 shows Southern Blot results of cells after recombination using the CD94-5′ Probe, CD94-3′ Probe, and Neo Probe. 1-A09, 2-B09, 2-C02, 2-D02, 2-H10, 3-B12, 3-D05, 4-C03, and 4-G06 are clone numbers. WT is a wild-type control.

FIG. 5A shows PCR identification results of F1 generation mice by primers CD94-WT-F and CD94-WT-R. CD94-F1-1 is mouse number. M is a marker. PC is a positive control (Neo cassette not removed). WT is a wild-type control. H₂O is a water control.

FIG. 5B shows PCR identification results of F1 generation mice by primers CD94-WT-F and CD94-Mut-R. CD94-F1-1 is mouse number. M is a marker. PC is a positive control (Neo cassette not removed). WT is a wild-type control. H₂O is a water control.

FIG. 6A shows a flow cytometry result of spleen cells from wild-type C57BL/6 mice (WT). The spleen cells were stained with mNK1.1-PE/Cy7, FITC Rat Anti-Mouse CD3 antibody, and APC Rat IgG2a, κ Isotype Ctrl antibody.

FIG. 6B shows a flow cytometry result of spleen cells from wild-type C57BL/6 mice (WT). The spleen cells were stained with mNK1.1-PE/Cy7, FITC Rat Anti-Mouse CD3 antibody, and mCD94-APC.

FIG. 6C shows a flow cytometry result of spleen cells from CD94 gene humanized heterozygous mice (H/+). The spleen cells were stained with mNK1.1-PE/Cy7, FITC Rat Anti-Mouse CD3 antibody, and mCD94-APC.

FIG. 6D shows a flow cytometry result of spleen cells from wild-type C57BL/6 mice (WT). The spleen cells were stained with mNK1.1-PE/Cy7, FITC Rat Anti-Mouse CD3 antibody, and hCD94-PE.

FIG. 6E shows a flow cytometry result of spleen cells from CD94 gene humanized heterozygous mice (H/+). The spleen cells were stained with mNK1.1-PE/Cy7, FITC Rat Anti-Mouse CD3 antibody, and hCD94-PE.

FIG. 7A is a schematic diagram showing mouse NKG2A gene locus.

FIG. 7B is a schematic diagram showing human NKG2A gene locus.

FIG. 8 is a schematic diagram showing humanized NKG2A gene locus. A mouse NKG2A gene sequence starting from exon 2 and ending within exon 6 was replaced with a human NKG2A gene sequence starting from exon 4 and ending within exon 8.

FIG. 9 is a schematic diagram showing a NIG2A gene targeting strategy. The targeting vector includes a 5′ homologous arm, a 3′ homologous arm, and a knock-in (KI) fragment (including a human NKG2A gene DNA fragment and Neo cassette). ADTA sequence is included at the downstream of the 3′ homologous arm.

FIG. 10A shows sgRNA activity detection results of sgRNA1-sgRNA8. Con is a negative control. PC is a positive control.

FIG. 10B shows sgRNA activity detection results of sgRNA9-sgRNA15. Con is a negative control. PC is a positive control.

FIG. 11 shows RT-PCR identification results of wild-type C57BL/6 mice (+/+) or NKG2A/CD94 double-gene humanized homozygous mice (H/H) to detect expression of mouse CD94 (mCD94, target fragment size: 452 bp), human CD94 (hCD94, target fragment size: 288 bp), mouse NKG2A (mNKG2A, target fragment size: 661 bp), human NKG2A (hNKG2A, target fragment size: 245 bp), and human GAPDH (target fragment size: 479 bp). H₂O is a water control.

FIG. 12A shows a flow cytometry result of spleen cells from wild-type C57BL/6 mouse (WT). The spleen cells were stained with an isotype control antibody (ISO) and mNK1.1-PE/Cy7.

FIG. 12B shows a flow cytometry result of spleen cells from B-hCD94/hNKG2A mouse (H/H). The spleen cells were stained with an isotype control antibody (ISO) and mNK1.1-PE/Cy7.

FIG. 12C shows a flow cytometry result of spleen cells from wild-type C57BL/6 mouse (WT). The spleen cells were stained with mCD94-APC and mNK1.1-PE/Cy7.

FIG. 12D shows a flow cytometry result of spleen cells from B-hCD94/hNKG2A mouse (H/H). The spleen cells were stained with mCD94-APC and mNK1.1-PE/Cy7.

FIG. 12E shows a flow cytometry result of spleen cells from wild-type C57BL/6 mouse (WT). The spleen cells were stained with an isotype control antibody (ISO) and mNK1.1-PE/Cy7.

FIG. 12F shows a flow cytometry result of spleen cells from B-hCD94/hNKG2A mouse (H/H). The spleen cells were stained with an isotype control antibody (ISO) and mNK1.1-PE/Cy7.

FIG. 12G shows a flow cytometry result of spleen cells from wild-type C57BL/6 mouse (WT). The spleen cells were stained with hCD94-PE and mNK1.1-PE/Cy7.

FIG. 12H shows a flow cytometry result of spleen cells from B-hCD94/hNKG2A mouse (H/H). The spleen cells were stained with hCD94-PE and mNK1.1-PE/Cy7.

FIG. 13A shows a flow cytometry result of spleen cells from wild-type C57BL/6 mouse (WT). The spleen cells were stained with an isotype control antibody (ISO) and mNK1.1-PE/Cy7.

FIG. 13B shows a flow cytometry result of spleen cells from B-hCD94/hNKG2A mouse (H/H). The spleen cells were stained with an isotype control antibody (ISO) and mNK1.1-PE/Cy7.

FIG. 13C shows a flow cytometry result of spleen cells from wild-type C57BL/6 mouse (WT). The spleen cells were stained with mNKG2A-PE and mNK1.1-PE/Cy7.

FIG. 13D shows a flow cytometry result of spleen cells from B-hCD94/hNKG2A mouse (H/H). The spleen cells were stained with mNKG2A-PE and mNK1.1-PE/Cy7.

FIG. 13E shows a flow cytometry result of spleen cells from wild-type C57BL/6 mouse (WT). The spleen cells were stained with Human IgG4, κ Isotype Control (ISO) in combination with Alexa Fluor® 647-conjugated AffiniPure F(ab′)2 Fragment Goat Anti-Human IgG, Fcy fragment Specific (AF647); and mNK1.1-PE/Cy7.

FIG. 13F shows a flow cytometry result of spleen cells from B-hCD94/hNKG2A mouse (H/H). The spleen cells were stained with Human IgG4, κ Isotype Control (ISO) in combination with Alexa Fluor® 647-conjugated AffiniPure F(ab′)2 Fragment Goat Anti-Human IgG, Fcy fragment Specific (AF647); and mNK1.1-PE/Cy7.

FIG. 13G shows a flow cytometry result of spleen cells from wild-type C57BL/6 mouse (WT). The spleen cells were stained with monalizumab in combination with Alexa Fluor® 647-conjugated AffiniPure F(ab′)₂ Fragment Goat Anti-Human IgG, Fcy fragment Specific (AF647); and mNK1.1-PE/Cy7.

FIG. 13H shows a flow cytometry result of spleen cells from B-hCD94/hNKG2A mouse (H/H). The spleen cells were stained with monalizumab in combination with Alexa Fluor® 647-conjugated AffiniPure F(ab′)₂ Fragment Goat Anti-Human IgG, Fcy fragment Specific (AF647); and mNK1.1-PE/Cy7.

FIG. 14A shows a flow cytometry result of spleen cells from wild-type C57BL/6 mouse (WT). The spleen cells were stained with PE Mouse IgG2b, κ Isotype Ctrl Antibody (ISO) and mCD94-APC.

FIG. 14B shows a flow cytometry result of spleen cells from B-hCD94/hNKG2A mouse (H/H). The spleen cells were stained with PE Mouse IgG2b, κ Isotype Ctrl Antibody (ISO) and mCD94-APC.

FIG. 14C shows a flow cytometry result of spleen cells from wild-type C57BL/6 mouse (WT). The spleen cells were stained with mNKG2A-PE and mCD94-APC.

FIG. 14D shows a flow cytometry result of spleen cells from B-hCD94/hNKG2A mouse (H/H). The spleen cells were stained with mNKG2A-PE and mCD94-APC.

FIG. 14E shows a flow cytometry result of spleen cells from wild-type C57BL/6 mouse (WT). The spleen cells were stained with Human IgG4, κ Isotype Control (ISO) in combination with Alexa Fluor® 647-conjugated AffiniPure F(ab′)2 Fragment Goat Anti-Human IgG, Fcy fragment Specific (AF647); and hCD94-PE.

FIG. 14F shows a flow cytometry result of spleen cells from B-hCD94/hNKG2A mouse (H/H). The spleen cells were stained with Human IgG4, κ Isotype Control (ISO) in combination with Alexa Fluor® 647-conjugated AffiniPure F(ab′)2 Fragment Goat Anti-Human IgG, Fcy fragment Specific (AF647); and hCD94-PE.

FIG. 14G shows a flow cytometry result of spleen cells from wild-type C57BL/6 mouse (WT). The spleen cells were stained with monalizumab in combination with Alexa Fluor® 647-conjugated AffiniPure F(ab′)₂ Fragment Goat Anti-Human IgG, Fcy fragment Specific (AF647); and hCD94-PE.

FIG. 14H shows a flow cytometry result of spleen cells from B-hCD94/hNKG2A mouse (H/H). The spleen cells were stained with monalizumab in combination with Alexa Fluor® 647-conjugated AffiniPure F(ab′)₂ Fragment Goat Anti-Human IgG, Fcy fragment Specific (AF647); and hCD94-PE.

FIG. 15 shows percentages of leukocyte subtypes in the spleen of C57BL/6 wild-type mice and NKG2A/CD94 double-gene humanized homozygous mice (B-hCD94/hNKG2A), as determined by flow cytometry.

FIG. 16 shows percentages of T cell subtypes in the spleen of C57BL/6 wild-type mice and NKG2A/CD94 double-gene humanized homozygous mice (B-hCD94/hNKG2A), as determined by flow cytometry.

FIG. 17 shows the average body weight of NKG2A/CD94 double-gene humanized homozygous mice that were xenografted with B-CAG-hHLA-E MC38 cells, and then treated with anti-human NKG2A antibody monalizumab (G2). An equal volume of PBS was administered as a control (G1).

FIG. 18 shows the average body weight change of NKG2A/CD94 double-gene humanized homozygous mice that were xenografted with B-CAG-hHLA-E MC38 cells, and then treated with anti-human NKG2A antibody monalizumab (G2). An equal volume of PBS was administered as a control (G1).

FIG. 19 shows the average tumor volume of NKG2A/CD94 double-gene humanized homozygous mice that were xenografted with B-CAG-hHLA-E MC38 cells, and then treated with anti-human NKG2A antibody monalizumab (G2). An equal volume of PBS was administered as a control (G1).

FIG. 20A shows a flow cytometry result of spleen cells from a wild-type C57BL/6 mouse (+/+) that were stained with PE Mouse IgG2b, κ Isotype Ctrl Antibody as an isotype control (ISO).

FIG. 20B shows a flow cytometry result of spleen cells from a wild-type C57BL/6 mouse (+/+) that were stained with mNKG2A-PE.

FIG. 20C shows a flow cytometry result of spleen cells from a NKG2A gene humanized heterozygous mouse (B-hNKG2A H/+) that were stained with mNKG2A-PE.

FIG. 20D shows a flow cytometry result of spleen cells from a wild-type C57BL/6 mouse (+/+) that were stained with Alexa Fluor® 647 Mouse IgG1, κ Isotype Ctrl (FC) as an isotype control (ISO).

FIG. 20E shows a flow cytometry result of spleen cells from a wild-type C57BL/6 mouse (+/+) that were stained with monalizumab in combination of Alexa Fluor® 647-conjugated AffiniPure F(ab′)2 Fragment Goat Anti-Human IgG, Fcy fragment Specific.

FIG. 20F shows a flow cytometry result of spleen cells from a NKG2A gene humanized heterozygous mouse (B-hNKG2A H/+) that were stained with monalizumab in combination of Alexa Fluor® 647-conjugated AffiniPure F(ab′)2 Fragment Goat Anti-Human IgG, Fcy fragment Specific.

FIG. 21 shows the alignment between human CD94 amino acid sequence (NP_001337991.1; SEQ ID NO: 2) and mouse CD94 amino acid sequence (NP_034784.1; SEQ ID NO: 1).

FIG. 22 shows the alignment between human CD94 amino acid sequence (NP_001337991.1; SEQ ID NO: 2) and rat CD94 amino acid sequence (NP_036877.1; SEQ ID NO: 65).

FIG. 23 shows the alignment between human NKG2A amino acid sequence (NP_998823.1; SEQ ID NO: 29) and mouse NKG2A amino acid sequence (NP_001129540.1; SEQ ID NO: 28).

FIG. 24 shows the alignment between human NKG2A amino acid sequence (NP_998823.1; SEQ ID NO: 29) and rat NKG2A amino acid sequence (NP_001032518.1; SEQ ID NO: 66).

### DETAILED DESCRIPTION

This disclosure relates to transgenic non-human animal with human or chimeric (e.g., humanized) CD94, and methods of use thereof.

Natural killer (NK) cells play an important role in the innate immune response to infections and malignancies by directly killing pathogen-infected or transformed cells and producing cytokines and chemokines that help shape the immune response. NK cell activation is controlled by a number of activating and inhibitory receptors. Activating NK receptors recognize a variety of stress-induced molecules such as the human MICA and MICB molecules, human ULBP1-6 proteins, mouse Rae-1, H60, and MULTI family members, all of which are recognized by the activating NKG2D receptor. Some activating NK receptors directly recognize pathogen-encoded ligands. For example, the m157 glycoprotein expressed by MCMV is recognized by the activating Ly49H receptor and NKp46 interacts with influenza hemagglutinin. NK cells are prevented from attacking normal self cells by inhibitory receptors that are reactive to MHC class I.

CD94/NKG2 is a family of C-type lectin receptors which are expressed predominantly on the surface of NK cells and a subset of CD8+ T-lymphocyte. These receptors stimulate or inhibit cytotoxic activity of NK cells, therefore they are divided into activating and inhibitory receptors according to their function. CD94/NKG2 recognize non-classical MHC glycoproteins class I (HLA-E in human and Qa-1 molecules in mouse). CD94/NKG2 family includes seven members: NKG2A, B, C, D, E, F and H. Genes encoding these receptors are clustered in the natural killer complex (NKC) on human chromosome 12 and mouse chromosome 6 together with Clr (C-lectin related) genes.

NKG2 receptors are transmembrane proteins type II which dimerize with CD94 molecule. CD94 contains a short cytoplasmic domain and it is responsible for signal transduction. Therefore NKG2 receptors form disulfide bonded heterodimers.

NKG2A and NKG2B receptors transmit inhibitory signal. They contain two Immunoreceptor tyrosine-based inhibitory motives (ITIM) in their cytoplasmic tail that are defined by the sequence (I/L/V/S)xYxx(L/V), where “x” means any amino acid at a given position. If ITIM-bearing receptors engage their ligand, probably Src family kinase phosphorylates tyrosine residue, and this allows recruitment of the tyrosine phosphatase SHP-1, SHP-2 or SHIP. It leads to de-phosphorylation of tyrosine kinase’s substrates, which are involved in the activating cascades. As a result, NK cell activation is suppressed. By contrast, NKG2C (encoded by the KLRC2 gene), NKG2E and NKG2H are activating receptors.

NKG2A can interact with CD94 to form a heterodimer inhibitory receptor of the C-type lectin family, recognizing a non-classical MHC-I molecule, HLA-E, as ligand. CD94-NKG2A and its HLA-E ligand are non-polymorphic. HLA-E*0101 and HLA-E*0103 represent the only two alleles exhibited by HLA-E in worldwide populations. Almost 50% of the NK cells in the peripheral blood express CD94/NKG2A, primarily those that do not express inhibitory KIRs (killer cell immunoglobulin-like receptors). The co-expression of CD94/NKG2A with other inhibitory receptors of different specificity also exists. In addition, γδ and CD 8+ T cells also express CD94/NKG2A. Ligation of NKG2A and CD94 to HLA-E expressed on normal cells suppresses signaling activation, thereby avoiding the destruction of normal bystander cells.

Non-classical MHC glycoproteins class I are structurally similar to classical MHC class I molecules, but they present mainly peptides derived from the signal peptides of MHC class I. Therefore NK cells can indirectly monitor the expression of classical MHC class I molecules through the interaction of CD94/NKG2 with HLA-E (or Qa-1 in mouse) and HLA-E (or Qa-1 in mouse) themselves as well. During cytomegalovirus infection, virus peptides are presented on HLA-E and NK cells that express the CD94/NKG2C receptor can specifically recognize these virus peptides, which results in activation, expansion, and differentiation of adaptive NK cells. HLA-E molecules are expressed at low levels in most tissues and primarily present peptides derived from the leader sequences of classical class Ia HLA molecules. However, HLA-E is commonly expressed at high levels on the surface of a variety of different cancers.

Tumor cells (hematological as well as solid tumors), in order to avoid killing by NK cells, have shown upregulation of HLA-E expression. In various cancers, poor prognosis has been associated with HLA-E upregulation, including colorectal, ovarian, gynecologic cancers, liver, glioblastoma, Hodgkin lymphoma, chronic lymphocytic leukemia, esophagus, gastric, pancreas, colon, kidney, head and neck, lung and melanoma. Blocking of the CD94/NKG2A receptor with an antibody could be used as a therapeutic strategy. Hence, an antibody against CD94/NKG2A (IPH2201-Monalizumab), developed by Innate Pharma, has been employed in various trials. Thus, CD94 or NKG2A antibodies can be potentially useful as cancer therapies.

Experimental animal models are an indispensable research tool for studying the effects of these antibodies (e.g., CD94 or NKG2A antibodies). Common experimental animals include mice, rats, guinea pigs, hamsters, rabbits, dogs, monkeys, pigs, fish and so on. However, there are many differences between human and animal genes and protein sequences, and many human proteins cannot bind to the animal’s homologous proteins to produce biological activity, leading to that the results of many clinical trials do not match the results obtained from animal experiments. A large number of clinical studies are in urgent need of better animal models. With the continuous development and maturation of genetic engineering technologies, the use of human cells or genes to replace or substitute an animal’s endogenous similar cells or genes to establish a biological system or disease model closer to human, and establish the humanized experimental animal models (humanized animal model) has provided an important tool for new clinical approaches or means. In this context, the genetically engineered animal model, that is, the use of genetic manipulation techniques, the use of human normal or mutant genes to replace animal homologous genes, can be used to establish the genetically modified animal models that are closer to human gene systems. The humanized animal models have various important applications. For example, due to the presence of human or humanized genes, the animals can express or express in part of the proteins with human functions, so as to greatly reduce the differences in clinical trials between humans and animals, and provide the possibility of drug screening at animal levels.

Unless otherwise specified, the practice of the methods described herein can take advantage of the techniques of cell biology, cell culture, molecular biology, transgenic biology, microbiology, recombinant DNA and immunology. These techniques are explained in detail in the following literature, for examples: Molecular Cloning A Laboratory Manual, 2nd Ed., ed. By Sambrook, Fritsch and Maniatis (Cold Spring Harbor Laboratory Press: 1989); DNA Cloning, Volumes I and II (D. N. Glovered., 1985); Oligonucleotide Synthesis (M. J. Gaited., 1984); Mullisetal U. S. Pat. No.4, 683, 195; Nucleic Acid Hybridization (B. D. Hames& S. J. Higginseds. 1984); Transcription And Translation (B. D. Hames& S. J. Higginseds. 1984); Culture Of Animal Cell (R. I. Freshney, Alan R. Liss, Inc., 1987); Immobilized Cells And Enzymes (IRL Press, 1986); B. Perbal, A Practical Guide To Molecular Cloning (1984), the series, Methods In ENZYMOLOGY (J. Abelson and M. Simon, eds.-in-chief, Academic Press, Inc., New York), specifically, Vols.154 and 155 (Wuetal. eds.) and Vol. 185, “Gene Expression Technology” (D. Goeddel, ed.); Gene Transfer Vectors For Mammalian Cells (J. H. Miller and M. P. Caloseds., 1987, Cold Spring Harbor Laboratory); Immunochemical Methods In Cell And Molecular Biology (Mayer and Walker, eds., Academic Press, London, 1987); Hand book Of Experimental Immunology, Volumes V (D. M. Weir and C. C. Blackwell, eds., 1986); and Manipulating the Mouse Embryo, (Cold Spring Harbor Laboratory Press, Cold Spring Harbor, N. Y., 1986); each of which is incorporated herein by reference in its entirety.

### CD94 (KLRD1)

CD94 (Cluster of Differentiation 94), also known as killer cell lectin-like receptor subfamily D, member 1 (KLRD1), is a type II transmembrane protein that is involved in cell signaling and is expressed on the surface of natural killer cells in the innate immune system. CD94 exists primarily in a heterodimeric form with NKG2A, C, and E on the cell surface. Depending on the associated NKG2 subunit, CD94/NKG2 can function as either an activating (NKG2C, and E) or an inhibitory (NKG2A) receptor. The inhibitory form has intracellular immunoreceptor tyrosine-based inhibitory motifs (ITIM), while the activating forms contain a positive transmembrane charged residue which facilitates interaction and signaling through the ITAM-containing DAP 12 molecule. Human Leukocyte Antigen-E (HLA-E) was identified as the ligand for CD94/NKG2.

The CD94 transmembrane-anchored glycoprotein forms disulfide-bonded heterodimers with the NKG2A subunit to form an inhibitory receptor or with the NKG2C or NKG2E subunits to assemble a receptor complex with activating DAP12 signaling proteins. CD94 receptors expressed on human and mouse NK cells and T cells have been proposed to be important in NK cell tolerance to self, play an important role in NK cell development, and contribute to NK cell-mediated immunity to certain infections including human cytomegalovirus.

In mice, the inhibitory members of the Ly49 C-type lectin-like receptor family of NK cell receptors are the primary MHC class I receptors. Humans do not express Ly49 receptors; instead, human NK cells express the structurally unrelated inhibitory killer cell immunoglobulin-like receptors (KIRs) that bind to MHC class I. Additionally, humans and mice both express members of the NKG2 family receptors that form obligate disulfide-bonded heterodimers with CD94. When expressed at physiological levels, the human NKG2 proteins cannot be stably expressed on the cell surface without CD94. CD94-NKG2 receptors bind non-classical MHC class Ib molecules, HLA-E in humans and Qa-1 in mice. HLA-E and Qa-1 both present conserved peptides derived from the leader segments of classical MHC class I molecules. Both human and murine NKG2 families consist of three members that share a high degree of similarity in their extracellular domains, NKG2A, NKG2C, and NKG2E. NKG2D is an unrelated receptor that does not pair with CD94 and has low sequence homology with NKG2A, NKG2C, and NKG2E.

In adult mice, CD94 is expressed on splenic NK cells and NKT cells and a small proportion of γδ T cells and CD8+ T cells, primarily CD44^{hi} memory cells. In mouse NK cells, NKG2A is the predominant NKG2 family member with NKG2A mRNA being more prevalent than NKG2C and NKG2E transcripts. NKG2A is the only NKG2 family member expressed in mouse T cells. The cytoplasmic domain of NKG2A contains one canonical immunoreceptor tyrosine-based inhibitory motif (ITIM) in mice and two ITIMs in human. Accordingly, triggering of CD94-NKG2A suppresses NK cell functions. NKG2C and NKG2E lack known intracellular signaling domains, but instead contain a charged residue within their transmembrane domains that facilitate binding to the DAP12 signaling adapter molecule, which contains an immunoreceptor tyrosine based activating motif (ITAM). Triggering of CD94-NKG2C leads to phosphorylation of the DAP12 ITAM and signaling via Syk and ZAP-70, resulting in NK cell activation. As with other paired inhibitory and activating ligands, the affinity for HLA-E is 10-fold higher for the inhibitory human CD94-NKG2A than the activating CD94-NKG2C receptor.

A detailed description of CD94 and its function can be found, e.g., in Orr, Mark T., et al., “Development and function of CD94-deficient natural killer cells.” PloS One 5.12 (2010): e15184; Joyce, M. G. et al., “The structural basis of ligand recognition by natural killer cell receptors.” Journal of Biomedicine and Biotechnology 2011 (2011); each of which is incorporated by reference in its entirety.

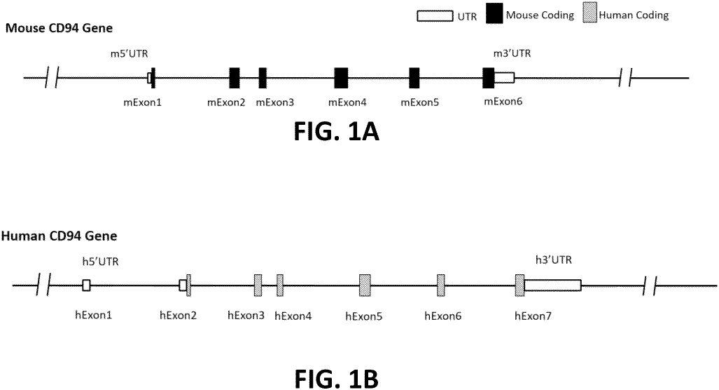

In human genomes, CD94 gene (Gene ID: 80381) locus has seven exons, exon 1, exon 2, exon 3, exon 4, exon 5, exon 6, and exon 7 (FIG. 1B). The CD94 protein also has a cytoplasmic region, a transmembrane region, and an extracellular region. The nucleotide sequence for human CD94 mRNA is NM_001351062.1, and the amino acid sequence for human CD94 is NP_001337991.1 (SEQ ID NO: 2). The location for each exon and each region in human CD94 nucleotide sequence and amino acid sequence is listed below:

TABLE 1

| Human CD94 (approximate location) | NM_001351062.1 3171bp | NP_001337991.1 179aa (SEQ ID NO: 2) |
|---|---|---|
| Exon 1 | 1..73 | Non-coding |
| Exon 2 | 74..180 | 1-2 |
| Exon 3 | 181..273 | 3-33 |
| Exon 4 | 274..336 | 34-54 |
| Exon 5 | 337..488 | 55-105 |
| Exon 6 | 489..592 | 106-140 |
| Exon 7 | 593..3171 | 141-179 |
| Cytoplasmic region | 174-203 | 1-10 |
| Transmembrane region | 204-266 | 11-31 |
| Extracellular region | 267-710 | 32-179 |
| Donor region in Example | 282-710 | 37-179 |

The human CD94 gene (Gene ID: 3824) is located in Chromosome 12 of the human genome, which is located from 10238383 to 10329608 of NC_000012.12. The 5′-UTR is from 10304446 to 10304547, and from 10307978 to 10308077, , Exon 1 is from 10304446 to 10304547, the first intron is from 10304548 to 10307977, Exon 2 is from 10307978 to 10308084, the second intron is from 10308085 to 10309387, Exon 3 is from 10309388 to 10309480, the third intron is from 10309481 to 10309625, Exon 4 is from 10309626 to 10309688, the fourth intron is from 10309689 to 10311463, Exon 5 is from 10311464 to 10311615, the fifth intron is from 10311616 to 10313409, Exon 6 is from 10313410 to 10313513, the sixth intron is from 10313514 to 10314672, Exon 7 is from 10314673 to 10329600, and the 3′-UTR is from 10314794 to 10329600. All relevant information for human CD94 locus can be found in the NCBI website with Gene ID: 3824, which is incorporated by reference herein in its entirety.

According to the UniProt Database (UniProt ID: Q13241), the cytoplasmic region of human CD94 corresponds to amino acids 1-10 of SEQ ID NO: 2; the transmembrane region of human CD94 corresponds to amino acids 11-31 of SEQ ID NO: 2; and the extracellular region of human CD94 corresponds to amino acids 32-179 of SEQ ID NO: 2. Specifically, there is a C-type lectin domain within the extracellular region of human CD94, which corresponds to amino acids 68-175 of SEQ ID NO: 2.

In mice, CD94 gene locus has six exons, exon 1, exon 2, exon 3, exon 4, exon 5, and exon 6 (FIG. 1A). The mouse CD94 protein also has a cytoplasmic region, a transmembrane region, and an extracellular region. The nucleotide sequence for mouse CD94 cDNA is NM_010654.4, the amino acid sequence for mouse CD94 is NP_034784.1 (SEQ ID NO: 1). The location for each exon and each region in the mouse CD94 nucleotide sequence and amino acid sequence is listed below:

TABLE 2

| | | |
|---|---|---|
| Mouse CD94 (approximate location) | NM_010654.4 867 bp | NP_034784.1 179 aa (SEQ ID NO: 1) |
| Exon 1 | 1..28 | 1-2 |
| Exon 2 | 29..121 | 3-33 |
| Exon 3 | 122..184 | 34-54 |
| Exon 4 | 185..336 | 55-105 |
| Exon 5 | 337..440 | 106-140 |
| Exon 6 | 441..867 | 141-179 |
| Cytoplasmic region | 22-51 | 1-10 |
| Transmembrane region | 52-114 | 11-31 |
| Extracellular region | 115-558 | 32-179 |
| Replaced region in | 130-558 | 37-179 |
| Example | | |

The mouse CD94 gene (Gene ID: 16643) is located in Chromosome 6 of the mouse genome, which is located from 129588092 to 129598775 of NC_000072.6 (GRCm38.p6 (GCF_000001635.26)). The 5′-UTR is from 129,591,782 to 129,591,830, exon 1 is from 129,591,782 to 129,591,837, the first intron is from 129,591,838 to 129,593,489, exon 2 is from 129,593,490 to 129,593,582, the second intron is from 129,593,583 to 129,593,727, exon 3 is from 129,593,728 to 129,593,790, the third intron is from 129,593,791 to 129,595,409, exon 4 is from 129,595,410 to 129,595,561, the fourth intron is from 129,595,562 to 129,596,861, exon 5 is from 129,596,862 to 129,596,965, the fifth intron is from 129,596,966 to 129,598,348, exon 6 is from 129,598,349 to 129,598,775, and the 3′-UTR is from 129598470 to 129,598,775, based on transcript NM_010654.4. All relevant information for mouse CD94 locus can be found in the NCBI website with Gene ID:16643, which is incorporated by reference herein in its entirety.

According to the UniProt Database (UniProt ID: 054707), the cytoplasmic region of mouse CD94 corresponds to amino acids 1-10 of SEQ ID NO: 1; the transmembrane region of mouse CD94 corresponds to amino acids 11-31 of SEQ ID NO: 1; and the extracellular region of mouse CD94 corresponds to amino acids 32-179 of SEQ ID NO: 1. Specifically, there is a C-type lectin domain within the extracellular region of mouse CD94, which corresponds to amino acids 68-175 of SEQ ID NO: 1.

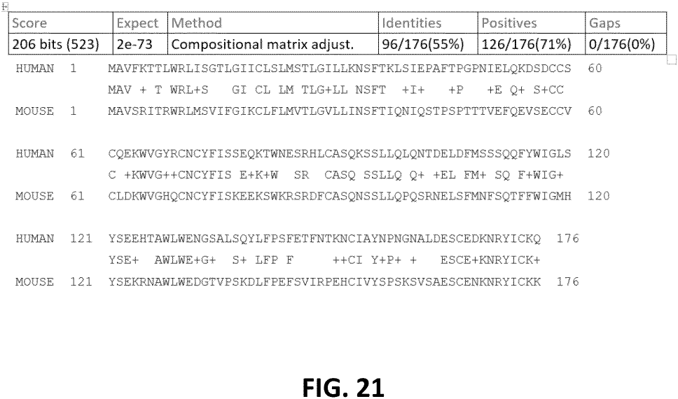

FIG. 21 shows the alignment between human CD94 amino acid sequence (NP_001337991.1; SEQ ID NO: 2) and mouse CD94 amino acid sequence (NP_034784.1; SEQ ID NO: 1). Thus, the corresponding amino acid residue or region between human and mouse CD94 can be found in FIG. 21.

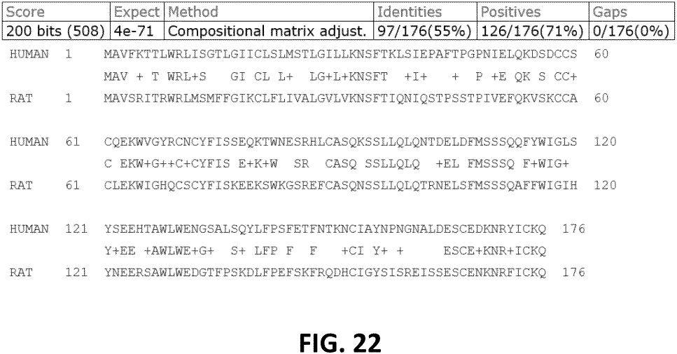

CD94 genes, proteins, and locus of the other species are also known in the art. For example, the gene ID for CD94 in Rattus norvegicus (rat) is 25110, the gene ID for CD94 in Macaca mulatta (Rhesus monkey) is 574145, the gene ID for CD94 in Canis lupus familiaris (dog) is 611360, and the gene ID for CD94 in Equus caballus (horse) is 100062540. The relevant information for these genes (e.g., intron sequences, exon sequences, amino acid residues of these proteins) can be found, e.g., in NCBI database, which is incorporated by reference herein in its entirety. FIG. 22 shows the alignment between human CD94 amino acid sequence (NP_001337991.1; SEQ ID NO: 2) and rodent CD94 amino acid sequence (NP_036877.1; SEQ ID NO: 65). Thus, the corresponding amino acid residue or region between human and rodent CD94 can be found in FIG. 22.

The present disclosure provides human or chimeric (e.g., humanized) CD94 nucleotide sequence and/or amino acid sequences. In some embodiments, the entire sequence of mouse exon 1, exon 2, exon 3, exon 4, exon 5, exon 6, cytoplasmic region, transmembrane region, extracellular region, and/or C-type lectin domain, are replaced by the corresponding human sequence. In some embodiments, a “region” or “portion” of mouse exon 1, exon 2, exon 3, exon 4, exon 5, exon 6, cytoplasmic region, transmembrane region, extracellular region, and/or C-type lectin domain are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 500, 600, 700, 800 nucleotides, or at least 1, 2, 3, 4, 5, 6, 7, 8, 9, 10, 20, 30, 40, 50, 60, 70, 80, 90, 100, 110, 120, 130, 140, 150, 160, 170, or 180 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, cytoplasmic region, transmembrane region, extracellular region, and/or C-type lectin domain of mouse CD94 gene or protein; or exon 1, exon 2, exon 3, exon 4, exon 5, exon 6, exon 7, cytoplasmic region, transmembrane region, extracellular region, and/or C-type lectin domain of human CD94 gene or protein. In some embodiments, a region, a portion, or the entire sequence of mouse CD94 exon 1, exon 2, exon 3, exon 4, exon 5, and/or exon 6 (e.g., a portion of exon 3, exons 4-5, and a portion of exon 6) are replaced by human CD94 exon 1, exon 2, exon 3, exon 4, exon 5, exon 6, and/or exon 7 (e.g., a portion of exon 4, exons 5-6, and a portion of exon 7) sequence.

In some embodiments, the genetically-modified non-human animal described herein comprises a sequence encoding a humanized CD94 protein. In some embodiments, the humanized CD94 protein comprises an endogenous cytoplasmic region. In some embodiments, the humanized CD94 protein comprises an endogenous transmembrane region. In some embodiments, the humanized CD94 protein comprises a humanized extracellular region. In some embodiments, the humanized CD94 protein comprises a humanized C-type lectin domain.

In some embodiments, the genetically-modified non-human animal described herein comprises a humanized CD94 gene. In some embodiments, the humanized CD94 gene comprises 6 exons. In some embodiments, the humanized CD94 gene comprises humanized exon 1, humanized exon 2, humanized exon 3, humanized exon 4, humanized exon 5, and/or humanized exon 6.

In some embodiments, the present disclosure also provides a chimeric (e.g., humanized) CD94 nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse CD94 mRNA sequence (e.g., NM_010654.4), mouse CD94 amino acid sequence (e.g., NP_034784.1; SEQ ID NO: 1), or a portion thereof (e.g., exons 1-2, a portion of exon 3, and a portion of exon 6 of mouse CD94 gene); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human CD94 mRNA sequence (e.g., NM_001351062.1), human CD94 amino acid sequence (e.g., NP_001337991.1; SEQ ID NO: 2), or a portion thereof (e.g., a portion of exon 4, exons 5-6, and a portion of exon 7 of human CD94 gene).

In some embodiments, the sequence encoding amino acids 37-179 of mouse CD94 (SEQ ID NO: 1) is replaced. In some embodiments, the sequence is replaced by a sequence encoding a corresponding region of human CD94 (e.g., amino acids 37-179 of human CD94 (SEQ ID NO: 2)).

In some embodiments, the sequence encoding amino acids 32-179 of mouse CD94 (SEQ ID NO: 1) is replaced. In some embodiments, the sequence is replaced by a sequence encoding a corresponding region of human CD94 (e.g., amino acids 32-179 of human CD94 (SEQ ID NO: 2)).

In some embodiments, the sequence encoding amino acids 68-175 of mouse CD94 (SEQ ID NO: 1) is replaced. In some embodiments, the sequence is replaced by a sequence encoding a corresponding region of human CD94 (e.g., amino acids 68-175 of human CD94 (SEQ ID NO: 2)).

In some embodiments, the sequence encoding the entirety or a portion of the extracellular region of mouse CD94 (SEQ ID NO: 1) is replaced or inactivated. In some embodiments, the sequence is replaced by a sequence encoding the entirety or a portion of the corresponding region of human CD94 (SEQ ID NO: 2). In some embodiments, the corresponding region of human CD94 comprises the entirety or a portion of the extracellular region of human CD94. In some embodiments, the sequence encoding amino acids 37-179 of mouse CD94 (SEQ ID NO: 1) is replaced. In some embodiments, the sequence is replaced by a sequence encoding a corresponding region of human CD94 (e.g., amino acids 37-179 of human CD94 (SEQ ID NO: 2)). In some embodiments, the sequence encoding amino acids 32-179 of mouse CD94 (SEQ ID NO: 1) is replaced. In some embodiments, the sequence is replaced by a sequence encoding a corresponding region of human CD94 (e.g., amino acids 32-179 of human CD94 (SEQ ID NO: 2)). In some embodiments, the sequence encoding the corresponding region of human CD94 does not include at least 1, at least 2, at least 3, at least 4, at least 5, at least 6, at least 7, at least 8, at least 9, or at least 10 amino acids at the N-terminus and/or C-terminus of the extracellular region of human CD94. In some embodiments, the sequence encoding the corresponding region of human CD94 comprises a nucleotide sequence within human CD94 exon 4 encoding at least 10-21, 10-15, or 16-19 contiguous amino acids. In some embodiments, the sequence encoding the corresponding region of human CD94 comprises a nucleotide sequence within human CD94 exon 4 encoding at least 20 or 21 contiguous amino acids. In some embodiments, the sequence encoding the corresponding region of human CD94 comprises a nucleotide sequence at the 3′ end of human CD94 exon 3 encoding at least 1, at least 2, at least 3, at least 4, or at least 5 amino acids.

In some embodiments, the human portion of the humanized CD94 protein may also include amino acid residues outside of 37-179 of SEQ ID NO: 2, which are identical or similar to the corresponding residues of endogenous CD94 protein of the non-human animal. Such residues can be derived from the extracellular region, transmembrane region, and/or cytoplasmic region, e.g., amino acids 34-179, or amino acids 33-179 of SEQ ID NO: 2, within the extracellular region of human CD94 protein. An alignment of the amino acid resides between human and mouse CD94 is shown in FIG. 21. The humanized CD94 proteins described herein can still be recognized by anti-human CD94 antibodies.

In some embodiments, the sequence encoding the extracellular C-type lectin domain of mouse CD94 (SEQ ID NO: 1) is replaced or inactivated. In some embodiments, the sequence is replaced by a sequence encoding the extracellular C-type lectin domain of human CD94 (SEQ ID NO: 2). In some embodiments, the sequence encoding amino acids 68-175 of mouse CD94 (SEQ ID NO: 1) is replaced. In some embodiments, the sequence is replaced by a sequence encoding amino acids 68-175 of human CD94 (SEQ ID NO: 2).

In some embodiments, the nucleic acids as described herein are operably linked to a promotor or regulatory element, e.g., an endogenous mouse CD94 promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from a portion of or the entire mouse CD94 nucleotide sequence (e.g., a portion of exon 3, exons 4-5, and a portion of exon 6 of NM_010654.4).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire mouse CD94 nucleotide sequence (e.g., exons 1-2, a portion of exon 3, exons 4-5, and a portion of exon 6 of NM_010654.4).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a portion of or the entire human CD94 nucleotide sequence (e.g., exons 1-3, a portion of exon 4, and a portion of exon 7 of NM_001351062.1).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire human CD94 nucleotide sequence (e.g., a portion of exon 4, exons 5-6, and a portion of exon 7 of NM_001351062.1).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire mouse CD94 amino acid sequence (e.g., an amino acid sequence encoded by a portion of exon 3, exons 4-5, and a portion of exon 6 of NM_010654.4; or amino acids 37-179 of NP_034784.1 (SEQ ID NO: 1)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire mouse CD94 amino acid sequence (e.g., an amino acid sequence encoded by exons 1-2, a portion of exon 3, exons 4-5, and a portion of exon 6 of NM_010654.4; or amino acids 1-36 of NP_034784.1 (SEQ ID NO: 1)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire human CD94 amino acid sequence (e.g., an amino acid sequence encoded by exons 1-3, a portion of exon 4, and a portion of exon 7 of NM_001351062.1; or amino acids 1-36 of NP_001337991.1 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire human CD94 amino acid sequence (e.g., an amino acid sequence encoded by a portion of exon 4, exons 5-6, and a portion of exon 7 of NM_001351062.1; or amino acids 37-179 of NP_001337991.1 (SEQ ID NO: 2)).

### NKG2A (KLRC1)

NKG2A (also known as CD159A), also known as killer cell lectin-like receptor subfamily C, member 1 (KLRC1), is a type II transmembrane protein belonging to the CD94/NKG2 family of C-type lectin-like receptors that inhibits innate immune system activation.

The cytoplasmic tail of human NKG2A receptor contains two immunoreceptor tyrosine-based inhibition motifs (ITIM) capable of recruiting both SHP-1 and SHP-2 phosphatases, but not the inositol phosphatase SHIP. Both ITIMs are required to mediate the maximal inhibitory signal, but the membrane-distal ITIM is of primary importance rather than the membrane-proximal ITIM. The partner CD94 has a short cytoplasmic region, thus lacks ITIMs and has no role in downstream signaling.

The CD94/NKG2A heterodimeric receptor is one of the most prominent NK inhibitory receptors. It binds to a nonclassical minimally polymorphic HLA class I molecule (HLA-E), which presents peptides derived from leader peptide sequences of other HLA class I molecules, such as HLA-G. Upon ligation by peptide-loaded HLA-E, NKG2A transduces inhibitory signaling through 2 inhibitory immune-receptor tyrosine-based inhibition motifs, thus suppressing NK cytokine secretion and cytotoxicity.

CD94/NKG2A and CD94/NKG2C were identified in the mid-1990s as cell surface glycoproteins that form disulfide-bonded heterodimers with CD94 and bind the non-classical MHC class Ib molecule HLA-E. NKG2C engagement in CMV-seropositive individuals imparts NK cell activation (with activated cells termed adaptive NK cells), while engagement of CD94/NKG2A transduces an inhibitory signal, consistent with the presence of two I/VxYxxL immunoreceptor tyrosine-based inhibitory motifs (ITIMs) within the cytoplasmic domain of NKG2A. While NKG2A expression and NKG2C expression are usually mutually exclusive, the frequency of NKG2A⁺ NK cells is considerably higher than that of NKG2C⁺ NK cells, especially in CMV-seronegative donors. Therefore, alongside killer immunoglobulin-like receptors (KIR), NKG2A represents a dominant inhibitory receptor on NK cells. The NKG2A phospho-ITIMs interact directly with the SH2 domains of the tyrosine phosphatases SHP-1 and SHP-2. One of the major targets of SHP-1-mediated dephosphorylation in NK cells is the guanine exchange factor and adaptor protein Vav1. Dephosphorylation of Vav1 prevents Rac1-dependent rearrangement of the actin cytoskeleton and amplification of activating signals. Engagement of CD94/NKG2A by HLA-E within inhibitory signaling clusters can also lead to the phosphorylation of the signaling adaptor protein Crk and disruption of actin-dependent signaling upstream of Vav1. NKG2A is uniformly high on immunoregulatory CD56^{bright} NK cells, whereas cytotoxic CD56^{dim} NK cells exhibit more heterogeneous expression, with a general decrease associated with terminal differentiation.

A detailed description of NKG2A and its function can be found, e.g., in Khan, M., et al., “NK cell-based immune checkpoint inhibition.” Frontiers in Immunology 11 (2020); Kamiya, T. et al., “Blocking expression of inhibitory receptor NKG2A overcomes tumor resistance to NK cells.” The Journal of Clinical Investigation 129.5 (2019): 2094-2106; Borst, L., et al., “The NKG2A-HLA-E axis as a novel checkpoint in the tumor microenvironment.” Clinical Cancer Research 26.21 (2020): 5549-5556; Cichocki, F. et al., “Setting traps for NKG2A gives NK cell immunotherapy a fighting chance.” The Journal of Clinical Investigation 129.5 (2019): 1839-1841; each of which is incorporated by reference in its entirety.

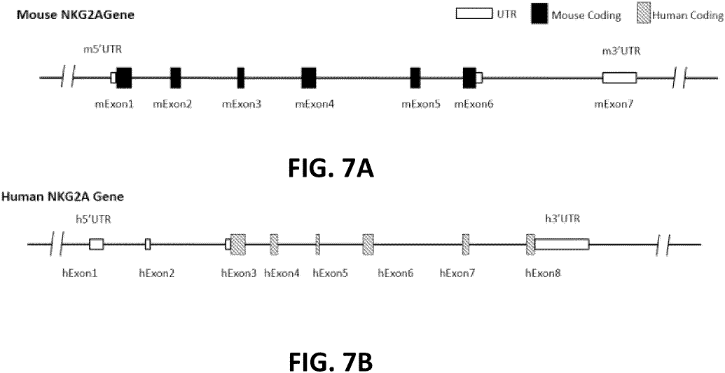

In human genomes, NKG2A gene (Gene ID: 3821) locus has eight exons, exon 1, exon 2, exon 3, exon 4, exon 5, exon 6, exon 7, and exon 8 (FIG. 7B). The NKG2A protein also has a cytoplasmic region, a transmembrane region, and an extracellular region. The nucleotide sequence for human NKG2A mRNA is NM_213658.2, and the amino acid sequence for human NKG2A is NP_998823.1 (SEQ ID NO: 29). The location for each exon and each region in human NKG2A nucleotide sequence and amino acid sequence is listed below:

TABLE 3

| Human NKG2A (approximate location) | NM_213658.2 1617bp | NP_998823.1 233aa SEQ ID NO: 29 |
|---|---|---|
| Exon 1 | 1..285 | Non-coding |
| Exon 2 | 286..357 | Non-coding |
| Exon 3 | 358..575 | 1-62 |
| Exon 4 | 576..671 | 63-94 |
| Exon 5 | 672..725 | 95-112 |
| Exon 6 | 726..877 | 113-163 |
| Exon 7 | 878..978 | 164-197 |
| Exon 8 | 979..1613 | 198-233 |
| Cytoplasmic region | 389-598 | 1-70 |
| Transmembrane region | 599-664 | 71-93 |
| Extracellular region | 665-1087 | 94-233 |
| Donor region in Example | 668-1087 | 94-233 |

The human NKG2A gene (Gene ID: 3821) is located in Chromosome 12 of the human genome, which is located from 10441673 to 10454685 of NC_000012.12.. The 5′-UTR is from 10454332 to 10454616, and from 10453198 to 10453269, and from 10451157 to 10451187, Exon 1 is from 10454616 to 10454332, the first intron is from 10454331 to 10453270, Exon 2 is from 10453269 to 10453198, the second intron is from 10453197 to 10451188, Exon 3 is from 10451187 to 10450970, the third intron is from 10450969 to 10450580, Exon 4 is from 10450579 to 10450484, the fourth intron is from 10450483 to 10449968, Exon 5 is from 10449967 to 10449914, the fifth intron is from 10449913 to 10449389, Exon 6 is from 10449388 to 10449237, the sixth intron is from 10449236 to 10447633, Exon 7 is from 10447632 to 10447532, the seventh intron is from 10447531 to 10446663, Exon 8 is from 10446662 to 10446041 and the 3′-UTR is from 10446041 to 10446550.All relevant information for human NKG2A locus can be found in the NCBI website with Gene ID: 3821, which is incorporated by reference herein in its entirety.

According to the UniProt Database (UniProt ID: P26715), the cytoplasmic region of human NKG2A corresponds to amino acids 1-70 of SEQ ID NO: 29; the transmembrane region of human NKG2A corresponds to amino acids 71-93 of SEQ ID NO: 29; and the extracellular region of human NKG2A corresponds to amino acids 94-233 of SEQ ID NO: 29. Specifically, there is a C-type lectin domain within the extracellular region of human NKG2A, which corresponds to amino acids 118-231 of SEQ ID NO: 29. In addition, there are two Immunoreceptor tyrosine-based inhibition motifs (ITIMs) within the cytoplasmic region, which correspond to amino acids 6-11 and 38-43 of SEQ ID NO: 29, respectively.

In mice, NKG2A gene locus has seven exons, exon 1, exon 2, exon 3, exon 4, exon 5, exon 6, and exon 7 (FIG. 7A). The mouse NKG2A protein also has a cytoplasmic region, a transmembrane region, and an extracellular region. The nucleotide sequence for mouse NKG2A cDNA is NM_001136068.2, the amino acid sequence for mouse NKG2A is NP_001129540.1 (SEQ ID NO: 28). The location for each exon and each region in the mouse NKG2A nucleotide sequence and amino acid sequence is listed below:

TABLE 4

| Mouse NKG2A (approximate location) | NM_001136068.2 1993 bp | NP_001129540.1 244 aa SEQ ID NO: 28 |
|---|---|---|
| Exon 1 | 1..250 | 1-62 |
| Exon 2 | 251..352 | 63-96 |
| Exon 3 | 353..403 | 97-113 |
| Exon 4 | 404..579 | 114-172 |
| Exon 5 | 580..680 | 173-206 |
| Exon 6 | 681..823 | 207-244 |
| Exon 7 | 824..1984 | Non-coding |
| Cytoplasmic region | 64-276 | 1-71 |
| Transmembrane region | 277-345 | 72-94 |
| Extracellular region | 346-795 | 95-244 |
| Replaced region in Example | 349-795 | 96-244 |

The mouse NKG2A gene (Gene ID: 16641) is located in Chromosome 6 of the mouse genome, which is located from 129666015 to 129682852 of NC_000072.6 (GRCm38.p6 (GCF_000001635.26)). The 5′-UTR is from 129,678,973 to 129,678,911, exon 1 is from 129,678,973 to 129,678,724, the first intron is from 129,678,723 to 129,678,398, exon 2 is from 129,678,397 to 129,678,296, the second intron is from 129,678,295 to 129,677,816, exon 3 is from 129,677,815 to 129,677,765, the third intron is from 129,677,764 to 129,677,323, exon 4 is from 129,677,322 to 129,677,147, the fourth intron is from 129,677,146 to 129,675,401, exon 5 is from 129,675,400 to 129,675,300, the fifth intron is from 129,675,299 to 129,674,977, exon 6 is from X129,674,976 to 129,674,834, the sixth intron is from 129,674,833 to 129,667,176, exon 7 is from 129,667,175 to 129,666,015, the 3′-UTR is from 129,674,858 to 129,674,834 and 129,667,175 to 129,666,015, based on transcript NM_001136068.2. All relevant information for mouse NKG2A locus can be found in the NCBI website with Gene ID: 16641, which is incorporated by reference herein in its entirety.

According to the UniProt Database (UniProt ID: Q9Z202), the cytoplasmic region of mouse NKG2A corresponds to amino acids 1-71 of SEQ ID NO: 28; the transmembrane region of mouse NKG2A corresponds to amino acids 72-94 of SEQ ID NO: 28; and the extracellular region of mouse NKG2A corresponds to amino acids 95-244 of SEQ ID NO: 28. Specifically, there is a C-type lectin domain within the extracellular region of mouse NKG2A, which corresponds to amino acids 135-239 of SEQ ID NO: 28.

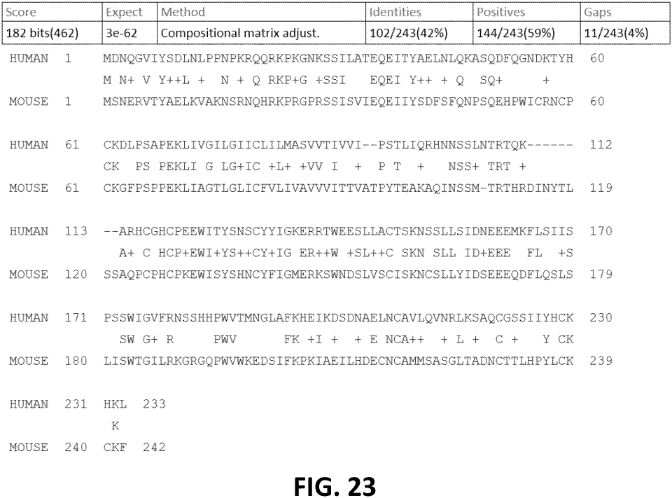

FIG. 23 shows the alignment between human NKG2A amino acid sequence (NP_998823.1; SEQ ID NO: 29) and mouse NKG2A amino acid sequence (NP_001129540.1; SEQ ID NO: 28). Thus, the corresponding amino acid residue or region between human and mouse NKG2A can be found in FIG. 23.

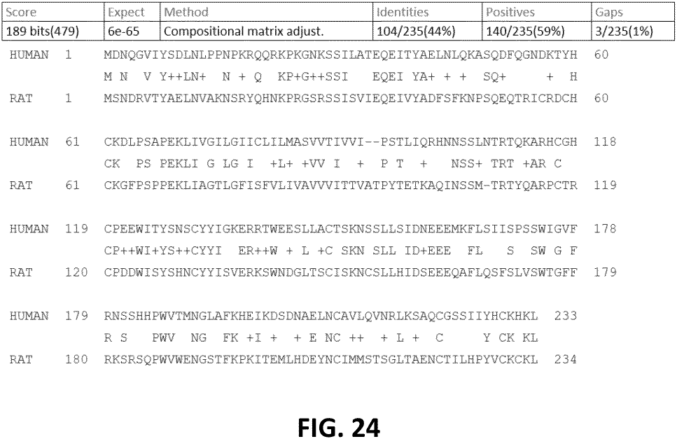

NKG2A genes, proteins, and locus of the other species are also known in the art. For example, the gene ID for NKG2A in Rattus norvegicus (rat) is 29683, the gene ID for NKG2A in Macaca mulatta (Rhesus monkey) is 574146, the gene ID for NKG2A in Bos taurus (cattle) is 100139049, and the gene ID for NKG2A in Pan troglodytes (chimpanzee) is 450131. The relevant information for these genes (e.g., intron sequences, exon sequences, amino acid residues of these proteins) can be found, e.g., in NCBI database, which is incorporated by reference herein in its entirety. FIG. 24 shows the alignment between human NKG2A amino acid sequence (NP_998823.1; SEQ ID NO: 29) and rodent NKG2A amino acid sequence (NP_001032518.1; SEQ ID NO: 66). Thus, the corresponding amino acid residue or region between human and rodent NKG2A can be found in FIG. 24.

The present disclosure provides human or chimeric (e.g., humanized) NKG2A nucleotide sequence and/or amino acid sequences. In some embodiments, the entire sequence of mouse exon 1, exon 2, exon 3, exon 4, exon 5, exon 6, exon 7, cytoplasmic region, transmembrane region, extracellular region, and/or C-type lectin domain, are replaced by the corresponding human sequence. In some embodiments, a “region” or “portion” of mouse exon 1, exon 2, exon 3, exon 4, exon 5, exon 6, exon 7, cytoplasmic region, transmembrane region, extracellular region, and/or C-type lectin domain are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 500, 600, 700, 800, 900, 1000, 1100, 1200, 1300, 1400, 1500, 1600, 1700, 1800, 1900 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, or 240 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, cytoplasmic region, transmembrane region, extracellular region, and/or C-type lectin domain of mouse NKG2A gene or protein; or exon 1, exon 2, exon 3, exon 4, exon 5, exon 6, exon 7, exon 8, cytoplasmic region, transmembrane region, extracellular region, and/or C-type lectin domain of human NKG2A gene or protein. In some embodiments, a region, a portion, or the entire sequence of mouse NKG2A exon 1, exon 2, exon 3, exon 4, exon 5, exon, 6, and/or exon 7 (e.g., a portion of exon 2, exons 3-5, and a portion of exon 6) are replaced by human NKG2A exon 1, exon 2, exon 3, exon 4, exon 5, exon 6, exon 7, and/or exon 8 (e.g., a portion of exon 4, exons 5-7, and a portion of exon 8) sequence.

In some embodiments, the genetically-modified non-human animal described herein comprises a sequence encoding a humanized NKG2A protein. In some embodiments, the humanized NKG2A protein comprises an endogenous cytoplasmic region. In some embodiments, the humanized NKG2A protein comprises an endogenous transmembrane region. In some embodiments, the humanized NKG2A protein comprises a humanized extracellular region. In some embodiments, the humanized NKG2A protein comprises a humanized C-type lectin domain.

In some embodiments, the genetically-modified non-human animal described herein comprises a humanized NKG2A gene. In some embodiments, the humanized NKG2A gene comprises 7 exons. In some embodiments, the humanized NKG2A gene comprises humanized exon 1, humanized exon 2, humanized exon 3, humanized exon 4, humanized exon 5, humanized exon 6, and/or humanized exon 7.

In some embodiments, the present disclosure also provides a chimeric (e.g., humanized) NKG2A nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse NKG2A mRNA sequence (e.g., NM_001136068.2), mouse NKG2A amino acid sequence (e.g., NP_001129540.1; SEQ ID NO: 28), or a portion thereof (e.g., exon 1, a portion of exon 2, a portion of exon 6, and exon 7 of mouse NKG2A gene); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human NKG2A mRNA sequence (e.g., NM_213658.2), human NKG2A amino acid sequence (e.g., NP_998823.1; SEQ ID NO: 29), or a portion thereof (e.g., a portion of exon 4, exons 5-7, and a portion of exon 8 of human NKG2A gene).

In some embodiments, the sequence encoding amino acids 96-244 of mouse NKG2A (SEQ ID NO: 28) is replaced. In some embodiments, the sequence is replaced by a sequence encoding a corresponding region of human NKG2A (e.g., amino acids 94-233 of human NKG2A (SEQ ID NO: 29)).

In some embodiments, the sequence encoding amino acids 95-244 of mouse NKG2A (SEQ ID NO: 28) is replaced. In some embodiments, the sequence is replaced by a sequence encoding a corresponding region of human NKG2A (e.g., amino acids 94-233 of human NKG2A (SEQ ID NO: 29)).

In some embodiments, the sequence encoding amino acids 135-239 of mouse NKG2A (SEQ ID NO: 28) is replaced. In some embodiments, the sequence is replaced by a sequence encoding a corresponding region of human NKG2A (e.g., amino acids 118-231 of human NKG2A (SEQ ID NO: 29)).

In some embodiments, the sequence encoding the entirety or a portion of the extracellular region of mouse NKG2A (SEQ ID NO: 28) is replaced or inactivated. In some embodiments, the sequence is replaced by a sequence encoding the entirety or a portion of the corresponding region of human NKG2A (SEQ ID NO: 29). In some embodiments, the corresponding region of human NKG2A comprises the entirety or a portion of the extracellular region of human NKG2A. In some embodiments, the sequence encoding amino acids 96-244 of mouse NKG2A (SEQ ID NO: 28) is replaced. In some embodiments, the sequence is replaced by a sequence encoding a corresponding region of human NKG2A (e.g., amino acids 94-233 of human NKG2A (SEQ ID NO: 29)). In some embodiments, the sequence encoding amino acids 95-244 of mouse NKG2A (SEQ ID NO: 28) is replaced. In some embodiments, the sequence is replaced by a sequence encoding a corresponding region of human NKG2A (e.g., amino acids 94-233 of human NKG2A (SEQ ID NO: 29)). In some embodiments, the sequence encoding the corresponding region of human NKG2A does not include at least 1, at least 2, at least 3, at least 4, at least 5, at least 6, at least 7, at least 8, at least 9, or at least 10 amino acids at the N-terminus and/or C-terminus of the extracellular region of human NKG2A. In some embodiments, the sequence encoding the corresponding region of human NKG2A comprises a nucleotide sequence at the 3′ end of human NKG2A exon 4 encoding at least 1, at least 2, at least 3, at least 4, at least 5, at least 6, at least 7, at least 8, at least 9, or at least 10 amino acids. In some embodiments, the sequence encoding the corresponding region of human NKG2A comprises a nucleotide sequence at the 3′ end of human NKG2A exon 4 encoding the last amino acid residue.

In some embodiments, the human portion of the humanized NKG2A protein may also include amino acid residues outside of 94-233 of SEQ ID NO: 29, which are identical or similar to the corresponding residues of endogenous NKG2A protein of the non-human animal. Such residues can be derived from the extracellular region, transmembrane region, and/or cytoplasmic region. An alignment of the amino acid resides between human and mouse NKG2A is shown in FIG. 23. The humanized NKG2A proteins described herein can still be recognized by anti-human NKG2A antibodies.

In some embodiments, the sequence encoding the extracellular C-type lectin domain of mouse NKG2A (SEQ ID NO: 28) is replaced or inactivated. In some embodiments, the sequence is replaced by a sequence encoding the extracellular C-type lectin domain of human NKG2A (SEQ ID NO: 29). In some embodiments, the sequence encoding amino acids 135-239 of mouse NKG2A (SEQ ID NO: 28) is replaced. In some embodiments, the sequence is replaced by a sequence encoding amino acids 118-231 of human NKG2A (SEQ ID NO: 29).

In some embodiments, the nucleic acids as described herein are operably linked to a promotor or regulatory element, e.g., an endogenous mouse NKG2A promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from a portion of or the entire mouse NKG2A nucleotide sequence (e.g., a portion of exon 2, exons 3-5, and a portion of exon 6 of NM_001136068.2).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire mouse NKG2A nucleotide sequence (e.g., exon 1, a portion of exon 2, a portion of exon 6, and exon 7 of NM_001136068.2).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a portion of or the entire human NKG2A nucleotide sequence (e.g., exons 1-3, a portion of exon 4, and a portion of exon 8 of NM_213658.2).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire human NKG2A nucleotide sequence (e.g., a portion of exon 4, exons 5-7, and a portion of exon 8 of NM_213658.2).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire mouse NKG2A amino acid sequence (e.g., an amino acid sequence encoded by a portion of exon 2, exons 3-5, and a portion of exon 6 of NM_001136068.2; or amino acids 96-244 of NP_001129540.1 (SEQ ID NO: 28)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire mouse NKG2A amino acid sequence (e.g., an amino acid sequence encoded by exon 1, a portion of exon 2, a portion of exon 6, and exon 7 of NM_001136068.2; or amino acids 1-95 of NP_001129540.1 (SEQ ID NO: 28)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire human NKG2A amino acid sequence (e.g., an amino acid sequence encoded by exons 1-3, a portion of exon 4, and a portion of exon 8 of NM_213658.2; or amino acids 1-93 of NP_998823.1 (SEQ ID NO: 29)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire human NKG2A amino acid sequence (e.g., an amino acid sequence encoded by a portion of exon 4, exons 5-7, and a portion of exon 8 of NM_213658.2; or amino acids 94-233 of NP_998823.1 (SEQ ID NO: 29)).

The present disclosure also provides a human or humanized CD94 amino acid sequence, or a human or humanized NKG2A amino acid sequence, wherein the amino acid sequence is selected from the group consisting of:
a) an amino acid sequence shown in SEQ ID NO: 1, 2, 8, 28, 29, or 35;
b) an amino acid sequence having a homology of at least 90% with or at least 90% identical to the amino acid sequence shown in SEQ ID NO: 1, 2, 8, 28, 29, or 35;
c) an amino acid sequence encoded by a nucleic acid sequence, wherein the nucleic acid sequence is able to hybridize to a nucleotide sequence encoding the amino acid shown in SEQ ID NO: 1, 2, 8, 28, 29, or 35 under a low stringency condition or a strict stringency condition;
d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 1, 2, 8, 28, 29, or 35;
e) an amino acid sequence that is different from the amino acid sequence shown in SEQ ID NO: 1, 2, 8, 28, 29, or 35 by no more than 10, 9, 8, 7, 6, 5, 4, 3, 2 or no more than 1 amino acid; or
f) an amino acid sequence that comprises a substitution, a deletion and /or insertion of one or more amino acids to the amino acid sequence shown in SEQ ID NO: 1, 2, 8, 28, 29, or 35.

The present disclosure also relates to a CD94 nucleic acid (e.g., DNA or RNA) sequence, or a NKG2A nucleic acid (e.g., DNA or RNA) sequence, wherein the nucleic acid sequence can be selected from the group consisting of:
a) a nucleic acid sequence as shown in SEQ ID NO: 3, 4, 5, 6, 7, 9, 10, 30, 31, 32, 33, 34, 36, or 37; a nucleic acid sequence encoding a homologous CD94 amino acid sequence of a humanized mouse; or a nucleic acid sequence encoding a homologous NKG2A amino acid sequence of a humanized mouse;
b) a nucleic acid sequence that is shown in SEQ ID NO: 3, 4, 5, 6, 7, 9, 10, 30, 31, 32, 33, 34, 36, or 37;
c) a nucleic acid sequence that is able to hybridize to the nucleotide sequence as shown in SEQ ID NO: 3, 4, 5, 6, 7, 9, 10, 30, 31, 32, 33, 34, 36, or 37 under a low stringency condition or a strict stringency condition;
d) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 3, 4, 5, 6, 7, 9, 10, 30, 31, 32, 33, 34, 36, or 37;
e) a nucleic acid sequence that encodes an amino acid sequence, wherein the amino acid sequence has a homology of at least 90% with or at least 90% identical to the amino acid sequence shown in SEQ ID NO: 1, 2, 8, 28, 29, or 35;
f) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 1, 2, 8, 28, 29, or 35;
g) a nucleic acid sequence that encodes an amino acid sequence, wherein the amino acid sequence is different from the amino acid sequence shown in SEQ ID NO: 1, 2, 8, 28, 29, or 35 by no more than 10, 9, 8, 7, 6, 5, 4, 3, 2 or no more than 1 amino acid; and/or
h) a nucleic acid sequence that encodes an amino acid sequence, wherein the amino acid sequence comprises a substitution, a deletion and /or insertion of one or more amino acids to the amino acid sequence shown in SEQ ID NO: 1, 2, 8, 28, 29, or 35.

The present disclosure also relates to a CD94 protein sequence, wherein the amino acid sequence of the CD94 protein can be selected from the group consisting of:
a) all or part of the amino acid sequence shown in SEQ ID NO: 8; or amino acids 37-179, 34-179, 33-179, 32-179, or 68-175 of SEQ ID NO: 2;
b) an amino acid sequence having a homology of at least 90%, 91%, 92%, 93%, 94%, 95%, 96%, 97%, 98%, or 99% or at least 90%, 91%, 92%, 93%, 94%, 95%, 96%, 97%, 98%, or 99% identical to the amino acid sequence shown in SEQ ID NO: 8; or amino acids 37-179, 34-179, 33-179, 32-179, or 68-175 of SEQ ID NO: 2;
c) an amino acid sequence that is different from the amino acid sequence shown in SEQ ID NO: 8; or amino acids 37-179, 34-179, 33-179, 32-179, or 68-175 of SEQ ID NO: 2, by no more than 10, 9, 8, 7, 6, 5, 4, 3, 2 or no more than 1 amino acid; and
d) an amino acid sequence that comprises a substitution, a deletion and/or insertion of one or more amino acids to the amino acid sequence shown in SEQ ID NO: 8; or amino acids 37-179, 34-179, 33-179, 32-179, or 68-175 of SEQ ID NO: 2.

The present disclosure also relates to a NKG2A protein sequence, wherein the amino acid sequence of the NKG2A protein can be selected from the group consisting of:
a) all or part of the amino acid sequence shown in SEQ ID NO: 35; or amino acids 94-233 or 118-231 of SEQ ID NO: 2;
b) an amino acid sequence having a homology of at least 90%, 91%, 92%, 93%, 94%, 95%, 96%, 97%, 98%, or 99% or at least 90%, 91%, 92%, 93%, 94%, 95%, 96%, 97%, 98%, or 99% identical to the amino acid sequence shown in SEQ ID NO: 35; or amino acids 94-233 or 118-231 of SEQ ID NO: 2;
c) an amino acid sequence that is different from the amino acid sequence shown in SEQ ID NO: 35; or amino acids 94-233 or 118-231 of SEQ ID NO: 2, by no more than 10, 9, 8, 7, 6, 5, 4, 3, 2 or no more than 1 amino acid; and
d) an amino acid sequence that comprises a substitution, a deletion and/or insertion of one or more amino acids to the amino acid sequence shown in SEQ ID NO: 35; or amino acids 94-233 or 118-231 of SEQ ID NO: 2.

The present disclosure also relates to a humanized CD94 gene or a humanized CD94 mRNA sequence, wherein the humanized CD94 mRNA sequence or the mRNA sequence transcribed from the humanized CD94 gene can be selected from the group consisting of:
a) all or part of the nucleotide sequence shown in SEQ ID NO: 7;
b) a nucleotide sequence that at least 70%, 80%, 90%, 91%, 92%, 93%, 94%, 95%, 96%, 97%, 98% or at least 99% identical to the nucleotide sequence shown in SEQ ID NO: 7;
c) a nucleotide sequence that is different from the nucleotide sequence shown in SEQ ID NO: 7 by no more than 10, 9, 8, 7, 6, 5, 4, 3, 2 or no more than 1 nucleotide; and
d) a nucleotide sequence that comprises a substitution, a deletion and/or insertion of one or more amino acids to the nucleotide sequence shown at SEQ ID NO: 7.

The present disclosure also relates to a humanized CD94 gene, wherein the humanized CD94 gene comprises a sequence that can be selected from the group consisting of:
a) all or part of the nucleotide sequence shown in SEQ ID NO: 5;
b) a nucleotide sequence that at least 70%, 80%, 90%, 91%, 92%, 93%, 94%, 95%, 96%, 97%, 98% or at least 99% identical to the nucleotide sequence shown in SEQ ID NO: 5;
c) a nucleotide sequence that is different from the nucleotide sequence shown in SEQ ID NO: 5 by no more than 10, 9, 8, 7, 6, 5, 4, 3, 2 or no more than 1 nucleotide; and
d) a nucleotide sequence that comprises a substitution, a deletion and/or insertion of one or more amino acids to the nucleotide sequence shown at SEQ ID NO: 5.

The present disclosure also relates to a humanized NKG2A gene or a humanized NKG2A mRNA sequence, wherein the humanized NKG2A mRNA sequence or the mRNA sequence transcribed from the humanized NKG2A gene can be selected from the group consisting of:
a) all or part of the nucleotide sequence shown in SEQ ID NO: 34;
b) a nucleotide sequence that at least 70%, 80%, 90%, 91%, 92%, 93%, 94%, 95%, 96%, 97%, 98% or at least 99% identical to the nucleotide sequence shown in SEQ ID NO: 34;
c) a nucleotide sequence that is different from the nucleotide sequence shown in SEQ ID NO: 34 by no more than 10, 9, 8, 7, 6, 5, 4, 3, 2 or no more than 1 nucleotide; and
d) a nucleotide sequence that comprises a substitution, a deletion and/or insertion of one or more amino acids to the nucleotide sequence shown at SEQ ID NO: 34.

The present disclosure also relates to a humanized NKG2A gene, wherein the humanized CD94 gene comprises a sequence that can be selected from the group consisting of:
a) all or part of the nucleotide sequence shown in SEQ ID NO: 32;
b) a nucleotide sequence that at least 70%, 80%, 90%, 91%, 92%, 93%, 94%, 95%, 96%, 97%, 98% or at least 99% identical to the nucleotide sequence shown in SEQ ID NO: 32;
c) a nucleotide sequence that is different from the nucleotide sequence shown in SEQ ID NO: 32 by no more than 10, 9, 8, 7, 6, 5, 4, 3, 2 or no more than 1 nucleotide; and
d) a nucleotide sequence that comprises a substitution, a deletion and/or insertion of one or more amino acids to the nucleotide sequence shown at SEQ ID NO: 32.

The present disclosure further relates to a humanized CD94 gene comprising a sequence that is at least 60%, at least 65%, at least 70%, at least 75%, at least 80%, at least 85%, at least 90%, at least 95%, or at least 99% identical to SEQ ID NO: 6.

The present disclosure further relates to a humanized NKG2A gene comprising a sequence that is at least 60%, at least 65%, at least 70%, at least 75%, at least 80%, at least 85%, at least 90%, at least 95%, or at least 99% identical to SEQ ID NO: 33.

The present disclosure further relates to a CD94 genomic DNA sequence of a humanized mouse. The DNA sequence (e.g., cDNA) is obtained by a reverse transcription of the mRNA obtained by transcription thereof and is consistent with or complementary to the DNA sequence homologous to the sequence shown in SEQ ID NO: 7.

The present disclosure further relates to a NKG2A genomic DNA sequence of a humanized mouse. The DNA sequence (e.g., cDNA) is obtained by a reverse transcription of the mRNA obtained by transcription thereof and is consistent with or complementary to the DNA sequence homologous to the sequence shown in SEQ ID NO: 34.

The disclosure also provides an amino acid sequence that has a homology of at least 90% with, or at least 90% identical to the sequence shown in SEQ ID NO: 1, 2, 8, 28, 29, or 35, and has protein activity. In some embodiments, the homology with the sequence shown in SEQ ID NO: 1, 2, 8, 28, 29, or 35 is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

In some embodiments, the percentage identity with the sequence shown in 1, 2, 8, 28, 29, or 35 is at least about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 5, 7, 32, or 34, and encodes a polypeptide that has protein activity. In some embodiments, the homology with the sequence shown in SEQ ID NO: 5, 7, 32, or 34 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

In some embodiments, the percentage identity with the sequence shown in SEQ ID NO: 5, 7, 32, or 34 is at least about 90%, 91%, 92%, 93%, 94%, 95%, 96%, 97%, 98%, or at least 99%. In some embodiments, the foregoing percentage identity is at least about 50%, 55%, 60%, 65%, 66%, 67%, 68%, 69%, 70%, 71%, 72%, 73%, 74%, 75%, 80%, or 85%.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For illustration purposes, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express human or chimeric (e.g., humanized) CD94 and/or NKG2A from an endogenous non-human CD94 or NKG2A locus.

### Genetically Modified Animals

As used herein, the term “genetically-modified non-human animal” refers to a non-human animal having exogenous DNA in at least one chromosome of the animal’s genome. In some embodiments, at least one or more cells, e.g., at least 1%, 2%, 3%, 4%, 5%, 10%, 20%, 30%, 40%, 50% of cells of the genetically-modified non-human animal have the exogenous DNA in its genome. The cell having exogenous DNA can be various kinds of cells, e.g., an endogenous cell, a somatic cell, an immune cell, a T cell, a B cell, an antigen presenting cell, a macrophage, a dendritic cell, a germ cell, a blastocyst, or an endogenous tumor cell. In some embodiments, genetically-modified non-human animals are provided that comprise a modified endogenous CD94 and/or NKG2A locus that comprises an exogenous sequence (e.g., a human sequence), e.g., a replacement of one or more non-human sequences with one or more human sequences. The animals are generally able to pass the modification to progeny, i.e., through germline transmission.

As used herein, the term “chimeric gene” or “chimeric nucleic acid” refers to a gene or a nucleic acid, wherein two or more portions of the gene or the nucleic acid are from different species, or at least one of the sequences of the gene or the nucleic acid does not correspond to the wildtype nucleic acid in the animal. In some embodiments, the chimeric gene or chimeric nucleic acid has at least one portion of the sequence that is derived from two or more different sources, e.g., sequences encoding different proteins or sequences encoding the same (or homologous) protein of two or more different species. In some embodiments, the chimeric gene or the chimeric nucleic acid is a humanized gene or humanized nucleic acid.

As used herein, the term “chimeric protein” or “chimeric polypeptide” refers to a protein or a polypeptide, wherein two or more portions of the protein or the polypeptide are from different species, or at least one of the sequences of the protein or the polypeptide does not correspond to wild-type amino acid sequence in the animal. In some embodiments, the chimeric protein or the chimeric polypeptide has at least one portion of the sequence that is derived from two or more different sources, e.g., same (or homologous) proteins of different species. In some embodiments, the chimeric protein or the chimeric polypeptide is a humanized protein or a humanized polypeptide.

As used herein, the term “humanized protein” or “humanized polypeptide” refers to a protein or a polypeptide, wherein at least a portion of the protein or the polypeptide is from the human protein or human polypeptide. In some embodiments, the humanized protein or polypeptide is a human protein or polypeptide.

As used herein, the term “humanized nucleic acid” refers to a nucleic acid, wherein at least a portion of the nucleic acid is from the human. In some embodiments, the entire nucleic acid of the humanized nucleic acid is from human. In some embodiments, the humanized nucleic acid is a humanized exon. A humanized exon can be e.g., a human exon or a chimeric exon.

In some embodiments, the chimeric gene or the chimeric nucleic acid is a humanized CD94 gene or a humanized CD94 nucleic acid. In some embodiments, at least one or more portions of the gene or the nucleic acid is from the human CD94 gene, at least one or more portions of the gene or the nucleic acid is from a non-human CD94 gene. In some embodiments, the gene or the nucleic acid comprises a sequence that encodes a CD94 protein. The encoded CD94 protein is functional or has at least one activity of the human CD94 protein or the non-human CD94 protein, e.g., binding with human or non-human NKG2 molecules (e.g., NKG2A or NKG2C); pairing with NKG2 molecules to interact with human or non-human MHC class I molecules (e.g., HLA-E in humans or Qa-1 in mice); activating or inhibiting NK cell and/or T cell activities; upregulating or downregulating the immune response.

In some embodiments, the chimeric gene or the chimeric nucleic acid is a humanized NKG2A gene or a humanized NKG2A nucleic acid. In some embodiments, at least one or more portions of the gene or the nucleic acid is from the human NKG2A gene, at least one or more portions of the gene or the nucleic acid is from a non-human NKG2A gene. In some embodiments, the gene or the nucleic acid comprises a sequence that encodes a NKG2A protein. The encoded NKG2A protein is functional or has at least one activity of the human NKG2A protein or the non-human NKG2A protein, e.g., binding with human or non-human CD94; pairing with CD94 to interact with human or non-human MHC class I molecules (e.g., HLA-E in humans or Qa-1 in mice); inhibiting NK cell or T cell activities; upregulating or downregulating the immune response.

In some embodiments, the MHC class I molecule described herein is a non-classic MHC class I molecule. In some embodiments, the MHC class I molecule described herein is a classic MHC class I molecule.

In some embodiments, the chimeric protein or the chimeric polypeptide is a humanized CD94 protein or a humanized CD94 polypeptide. In some embodiments, at least one or more portions of the amino acid sequence of the protein or the polypeptide is from a human CD94 protein, and at least one or more portions of the amino acid sequence of the protein or the polypeptide is from a non-human CD94 protein. The humanized CD94 protein or the humanized CD94 polypeptide is functional or has at least one activity of the human CD94 protein or the non-human CD94 protein.

In some embodiments, the chimeric protein or the chimeric polypeptide is a humanized NKG2A protein or a humanized NKG2A polypeptide. In some embodiments, at least one or more portions of the amino acid sequence of the protein or the polypeptide is from a human NKG2A protein, and at least one or more portions of the amino acid sequence of the protein or the polypeptide is from a non-human NKG2A protein. The humanized NKG2A protein or the humanized NKG2A polypeptide is functional or has at least one activity of the human NKG2A protein or the non-human NKG2A protein.

The genetically modified non-human animal can be various animals, e.g., a mouse, rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable embryonic stem (ES) cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In some embodiments, the non-human animal is a mouse.

In some embodiments, the animal is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the mouse is a 129 strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129S6 (129/SvEvTac), 129S7, 129S8, 129T1, 129T2. These mice are described, e.g., in Festing et al., Revised nomenclature for strain 129 mice, Mammalian Genome 10: 836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129).

In some embodiments, the animal is a rodent. In some embodiments, the rodent is selected from BALB/c, A, A/He, A/J, A/WySN, AKR, AKR/A, AKR/J, AKR/N, TA1, TA2, RF, SWR, C3H, C57BR, SJL, C57L, DBA/ 2. KM, NIH, ICR, CFW, FACA, C57BL/A, C57BL/An, C57BL/GrFa, C57BL/KaLwN, C57BL/6, C57BL/6J, C57BL/6ByJ, C57BL/6NJ, C57BL/10, C57BL/ 10ScSn, C57BL/10Cr and C57BL/Ola C57BL, C58, CBA/Br, CBA/Ca, CBA/J, CBA/st, CBA/H strains of mice and NOD, NOD/SCID, NOD-Prkdc^{scid} IL-2rg^{null} Background mice.

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the humanized CD94 and/or NKG2A animal is made. For example, suitable mice for maintaining a xenograft (e.g., a human cancer or tumor), can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes). Nonlimiting examples of such mice include, e.g., NOD-Prkdcscid fL-2ry^{null} NOD mice, NOD-Rag 1-/--IL2rg-/- (NRG) mice, Rag 2-/--IL2rg-/- (RG) mice, SCID mice, NOD/SCID mice, IL2Rγ knockout mice, NOD/SCID/γc^{null} mice (Ito, M. et al., NOD/SCID/ γcnull mouse: an excellent recipient mouse model for engraftment of human cells, Blood 100(9): 3175-3182, 2002), nude mice, and Rag1 and/or Rag2 knockout mice. These mice can optionally be irradiated, or otherwise treated to destroy one or more immune cell type. Thus, in various embodiments, a genetically modified mouse is provided that can include a humanization of at least a portion of an endogenous non-human CD94 and/or NKG2A locus, and further comprises a modification that compromises, inactivates, or destroys the immune system (or one or more cell types of the immune system) of the non-human animal in whole or in part. In some embodiments, modification is, e.g., selected from the group consisting of a modification that results in NOD-Prkdcscid IL-2rγ^{null} NOD mice, NOD-Rag 1-/--IL2rg-/- (NRG) mice, Rag 2-/--IL2rg-/- (RG) mice, NOD mice, SCID mice, NOD/SCID mice, IL-2Ry knockout mice, NOD/SCID/yc null mice, nude mice, Rag1 and/or Rag2 knockout mice, and a combination thereof. These genetically modified animals are described, e.g., in US20150106961, which is incorporated herein by reference in its entirety.

In some embodiments, the mouse can include a replacement of all or part of mature CD94 coding sequence with human mature CD94 coding sequence. In some embodiments, the mouse can include a replacement of all or part of mature NKG2A coding sequence with human mature NKG2A coding sequence.

In some embodiments, the genetically modified non-human animals comprises a modification of an endogenous non-human CD94 locus. In some embodiments, the modification can comprise a human nucleic acid sequence encoding at least a portion of a mature CD94 protein (e.g., at least 10%, 20%, 30%, 40%, 50%, 60%, 70%, 80%, 90%, 95%, 96%, 97%, 98%, or 99% identical to the mature CD94 protein sequence). Although genetically modified cells are also provided that can comprise the modifications described herein (e.g., ES cells, somatic cells), in many embodiments, the genetically modified non-human animals comprise the modification of the endogenous CD94 locus in the germline of the animal.

In some embodiments, the genetically modified mice express a human CD94 and/or a chimeric (e.g., humanized) CD94 from endogenous mouse loci, wherein the endogenous mouse CD94 gene has been replaced with a human CD94 gene and/or a nucleotide sequence that encodes a region of human CD94 sequence or an amino acid sequence that is at least 10%, 20%, 30%, 40%, 50%, 60%, 70&, 80%, 90%, 95%, 96%, 97%, 98%, or 99% identical to the human CD94 sequence. In various embodiments, an endogenous non-human CD94 locus is modified in whole or in part to comprise human nucleic acid sequence encoding at least one protein-coding sequence of a mature CD94 protein.

In some embodiments, the genetically modified mice express the human CD94 and/or chimeric CD94 (e.g., humanized CD94) from endogenous loci that are under control of mouse promoters and/or mouse regulatory elements. The replacement(s) at the endogenous mouse loci provide non-human animals that express human CD94 or chimeric CD94 (e.g., humanized CD94) in appropriate cell types and in a manner that does not result in the potential pathologies observed in some other transgenic mice known in the art. The human CD94 or the chimeric CD94 (e.g., humanized CD94) expressed in animal can maintain one or more functions of the wild-type mouse or human CD94 in the animal. For example, the expressed CD94 can interact with a human or non-human NKG2A, forming a heterodimer that can recognize MHC class I molecules. Furthermore, in some embodiments, the animal does not express endogenous CD94. As used herein, the term “endogenous CD94” refers to CD94 protein that is expressed from an endogenous CD94 nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to human CD94 (NP_001337991.1) (SEQ ID NO: 2). In some embodiments, the genome comprises a sequence encoding an amino acid sequence that is at least 70%, 75%, 80%, 85%, 90%, 95%, 99%, or 100% identical to SEQ ID NO: 8.

The genome of the genetically modified animal can comprise a replacement at an endogenous CD94 gene locus of a sequence encoding a region of endogenous CD94 with a sequence encoding a corresponding region of human CD94. In some embodiments, the sequence that is replaced is any sequence within the endogenous CD94 gene locus, e.g., exon 1, exon 2, exon 3, exon 4, exon 5, exon 6, 5′-UTR, 3′-UTR, the first intron, the second intron, and the third intron, the fourth intron, the fifth intron, etc. In some embodiments, the sequence that is replaced is within the regulatory region of the endogenous CD94 gene. In some embodiments, the sequence that is replaced starts within exon 3 and ends within exon 6 of an endogenous mouse CD94 gene locus.

The genetically modified animal can have one or more cells expressing a human or chimeric CD94 (e.g., humanized CD94) having a cytoplasmic region, a transmembrane region, and/or an extracellular region, wherein the extracellular region comprises a sequence that is at least 50%, 60%, 70%, 80%, 90%, 95%, 99% identical to the extracellular region of human CD94. In some embodiments, the extracellular region of the humanized CD94 has a sequence that has at least 10, 20, 30, 40, 50, 60, 70, 80, 90, 100, 110, 120, 130, or 140 amino acids (e.g., contiguously or non-contiguously) that are identical to human CD94.

Because human CD94 and non-human CD94 (e.g., mouse CD94) sequences, in many cases, are different, antibodies that bind to human CD94 will not necessarily have the same binding affinity with non-human CD94 or have the same effects to non-human CD94. Therefore, the genetically modified animal having a human or a humanized extracellular region can be used to better evaluate the effects of anti-human CD94 antibodies in an animal model. In some embodiments, the genome of the genetically modified animal comprises a sequence encoding an amino acid sequence that corresponds to part or the entire sequence of exons 4-7 of human CD94, part or the entire sequence of extracellular region of human CD94, or part or the entire sequence of amino acids 37-179, 34-179, 33-179, 32-179, or 68-175 of SEQ ID NO: 2.

In some embodiments, the non-human animal can have, at an endogenous CD94 gene locus, a nucleotide sequence encoding a chimeric human/non-human CD94 polypeptide, wherein a human portion of the chimeric human/non-human CD94 polypeptide comprises the entirety or a portion of human CD94 extracellular domain, and wherein the animal expresses a functional CD94 on a surface of a cell (e.g., NK cell or CD8+ T-lymphocyte) of the animal.

In some embodiments, the non-human portion of the chimeric human/non-human CD94 polypeptide comprises cytoplasmic and/or transmembrane regions of an endogenous non-human CD94 polypeptide. There may be several advantages that are associated with the cytoplasmic and/or transmembrane regions of an endogenous non-human CD94 polypeptide. For example, once MHC class I molecules (e.g., human HLA-E) binds to a NKG2A/CD94 receptor, or an anti-CD94 antibody binds to CD94, they can properly transmit extracellular signals into the cells and initiate the downstream pathway. A human or humanized transmembrane and/or cytoplasmic regions may not function properly in non-human animal cells. In some embodiments, a few (e.g., 1, 2, 3, 4, 5, 6, 7, 8, 9, or 10) extracellular amino acids that are close to the transmembrane region of CD94 are also derived from endogenous sequence.

In some embodiments, the genetically modified non-human animals comprises a modification of an endogenous non-human NKG2A locus. In some embodiments, the modification can comprise a human nucleic acid sequence encoding at least a portion of a mature NKG2A protein (e.g., at least 10%, 20%, 30%, 40%, 50%, 60%, 70%, 80%, 90%, 95%, 96%, 97%, 98%, or 99% identical to the mature NKG2A protein sequence). Although genetically modified cells are also provided that can comprise the modifications described herein (e.g., ES cells, somatic cells), in many embodiments, the genetically modified non-human animals comprise the modification of the endogenous NKG2A locus in the germline of the animal.

In some embodiments, the genetically modified mice express a human NKG2A and/or a chimeric (e.g., humanized) NKG2A from endogenous mouse loci, wherein the endogenous mouse NKG2A gene has been replaced with a human NKG2A gene and/or a nucleotide sequence that encodes a region of human NKG2A sequence or an amino acid sequence that is at least 10%, 20%, 30%, 40%, 50%, 60%, 70&, 80%, 90%, 95%, 96%, 97%, 98%, or 99% identical to the human NKG2A sequence. In various embodiments, an endogenous non-human NKG2A locus is modified in whole or in part to comprise human nucleic acid sequence encoding at least one protein-coding sequence of a mature NKG2A protein.

In some embodiments, the genetically modified mice express the human NKG2A and/or chimeric NKG2A (e.g., humanized NKG2A) from endogenous loci that are under control of mouse promoters and/or mouse regulatory elements. The replacement(s) at the endogenous mouse loci provide non-human animals that express human NKG2A or chimeric NKG2A (e.g., humanized NKG2A) in appropriate cell types and in a manner that does not result in the potential pathologies observed in some other transgenic mice known in the art. The human NKG2A or the chimeric NKG2A (e.g., humanized NKG2A) expressed in animal can maintain one or more functions of the wild-type mouse or human NKG2A in the animal. For example, the expressed NKG2A can interact with a human or non-human CD94, forming a heterodimer that can recognize MHC class I molecules. Furthermore, in some embodiments, the animal does not express endogenous NKG2A. As used herein, the term “endogenous NKG2A” refers to NKG2A protein that is expressed from an endogenous NKG2A nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to human NKG2A (NP_998823.1) (SEQ ID NO: 29). In some embodiments, the genome comprises a sequence encoding an amino acid sequence that is at least 70%, 75%, 80%, 85%, 90%, 95%, 99%, or 100% identical to SEQ ID NO: 35.

The genome of the genetically modified animal can comprise a replacement at an endogenous NKG2A gene locus of a sequence encoding a region of endogenous NKG2A with a sequence encoding a corresponding region of human NKG2A. In some embodiments, the sequence that is replaced is any sequence within the endogenous NKG2A gene locus, e.g., exon 1, exon 2, exon 3, exon 4, exon 5, exon 6, exon 7, 5′-UTR, 3′-UTR, the first intron, the second intron, and the third intron, the fourth intron, the fifth intron, the sixth intron, etc. In some embodiments, the sequence that is replaced is within the regulatory region of the endogenous NKG2A gene. In some embodiments, the sequence that is replaced starts within exon 2 and ends within exon 6 of an endogenous mouse NKG2A gene locus.

The genetically modified animal can have one or more cells expressing a human or chimeric NKG2A (e.g., humanized NKG2A) having a cytoplasmic region, a transmembrane region, and/or an extracellular region, wherein the extracellular region comprises a sequence that is at least 50%, 60%, 70%, 80%, 90%, 95%, 99% identical to the extracellular region of human NKG2A. In some embodiments, the extracellular region of the humanized NKG2A has a sequence that has at least 10, 20, 30, 40, 50, 60, 70, 80, 90, 100, 110, 120, 130, or 140 amino acids (e.g., contiguously or non-contiguously) that are identical to human NKG2A.

Because human NKG2A and non-human NKG2A (e.g., mouse NKG2A) sequences, in many cases, are different, antibodies that bind to human NKG2A will not necessarily have the same binding affinity with non-human NKG2A or have the same effects to non-human NKG2A. Therefore, the genetically modified animal having a human or a humanized extracellular region can be used to better evaluate the effects of anti-human NKG2A antibodies in an animal model. In some embodiments, the genome of the genetically modified animal comprises a sequence encoding an amino acid sequence that corresponds to part or the entire sequence of exons 4-8 of human NKG2A, part or the entire sequence of extracellular region of human NKG2A, or part or the entire sequence of amino acids 94-233 or 118-231 of SEQ ID NO: 29.

In some embodiments, the non-human animal can have, at an endogenous NKG2A gene locus, a nucleotide sequence encoding a chimeric human/non-human NKG2A polypeptide, wherein a human portion of the chimeric human/non-human NKG2A polypeptide comprises a portion of human NKG2A extracellular domain, and wherein the animal expresses a functional NKG2A on a surface of a cell (e.g., NK cell or CD8+ T-lymphocyte) of the animal.

In some embodiments, the non-human portion of the chimeric human/non-human NKG2A polypeptide comprises cytoplasmic and/or regions of an endogenous non-human NKG2A polypeptide. There may be several advantages that are associated with the cytoplasmic and/or transmembrane regions of an endogenous non-human NKG2A polypeptide. For example, once MHC class I molecules (e.g., human HLA-E) binds to a NKG2A/CD94 receptor, or an anti-NKG2A antibody binds to NKG2A, they can properly transmit extracellular signals into the cells and initiate the downstream pathway. A human or humanized transmembrane and/or cytoplasmic regions may not function properly in non-human animal cells. In some embodiments, a few (e.g., 1, 2, 3, 4, 5, 6, 7, 8, 9, or 10) extracellular amino acids that are close to the transmembrane region of NKG2A are also derived from endogenous sequence.

In some embodiments, the genetically modified animal does not express endogenous CD94. In some embodiments, the genetically modified animal expresses a decreased level of endogenous CD94 as compared to a wild-type animal. In some embodiments, the genetically modified animal does not express endogenous NKG2A. In some embodiments, the genetically modified animal expresses a decreased level of endogenous NKG2A as compared to a wild-type animal.

Furthermore, the genetically modified animal can be heterozygous with respect to the replacement at the endogenous CD94 locus, or homozygous with respect to the replacement at the endogenous CD94 locus. Furthermore, the genetically modified animal can be heterozygous with respect to the replacement at the endogenous NKG2A locus, or homozygous with respect to the replacement at the endogenous NKG2A locus.

In some embodiments, the humanized CD94 locus lacks a human CD94 5′-UTR. In some embodiment, the humanized CD94 locus comprises a rodent (e.g., mouse) 5′-UTR. In some embodiments, the humanization comprises a human 3′- UTR. In appropriate cases, it may be reasonable to presume that the mouse and human CD94 genes appear to be similarly regulated based on the similarity of their 5′-flanking sequence. As shown in the present disclosure, humanized CD94 mice that comprise a replacement at an endogenous mouse CD94 locus, which retain mouse regulatory elements but comprise a humanization of CD94 encoding sequence, do not exhibit pathologies. Both genetically modified mice that are heterozygous or homozygous for humanized CD94 are grossly normal.

In some embodiments, the humanized NKG2A locus lacks a human NKG2A 5′-UTR. In some embodiment, the humanized NKG2A locus comprises a rodent (e.g., mouse) 5′-UTR. In some embodiments, the humanization comprises a human 3′- UTR. In appropriate cases, it may be reasonable to presume that the mouse and human NKG2A genes appear to be similarly regulated based on the similarity of their 5′-flanking sequence. As shown in the present disclosure, humanized NKG2A mice that comprise a replacement at an endogenous mouse NKG2A locus, which retain mouse regulatory elements but comprise a humanization of NKG2A encoding sequence, do not exhibit pathologies. Both genetically modified mice that are heterozygous or homozygous for humanized NKG2A are grossly normal.

The present disclosure further relates to a non-human mammal generated through the method mentioned above. In some embodiments, the genome thereof contains human gene(s).

In some embodiments, the non-human mammal is a rodent, and preferably, the non-human mammal is a mouse.

In some embodiments, the non-human mammal expresses a protein encoded by a humanized CD94 gene. In some embodiments, the non-human mammal expresses a protein encoded by a humanized NKG2A gene.

In addition, the present disclosure also relates to a tumor bearing non-human mammal model, characterized in that the non-human mammal model is obtained through the methods as described herein. In some embodiments, the non-human mammal is a rodent (e.g., a mouse).

The present disclosure further relates to a cell or cell line, or a primary cell culture thereof derived from the non-human mammal or an offspring thereof, or the tumor bearing non-human mammal; the tissue, organ or a culture thereof derived from the non-human mammal or an offspring thereof, or the tumor bearing non-human mammal; and the tumor tissue derived from the non-human mammal or an offspring thereof when it bears a tumor, or the tumor bearing non-human mammal.

The present disclosure also provides non-human mammals produced by any of the methods described herein. In some embodiments, a non-human mammal is provided; and the genetically modified animal contains the DNA encoding human or humanized CD94 in the genome of the animal. The present disclosure also provides non-human mammals produced by any of the methods described herein. In some embodiments, a non-human mammal is provided; and the genetically modified animal contains the DNA encoding human or humanized NKG2A in the genome of the animal.

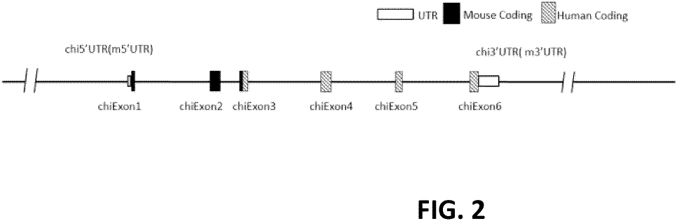

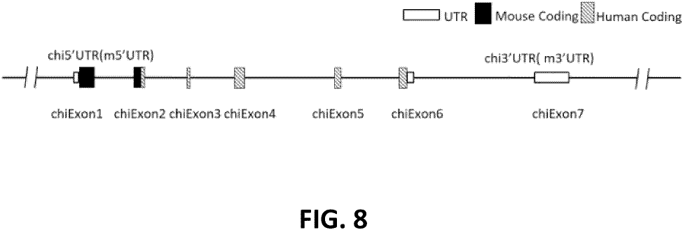

In some embodiments, the non-human mammal comprises the genetic construct as described herein (e.g., gene construct as shown in FIG. 2 or FIG. 8). In some embodiments, a non-human mammal expressing human or humanized CD94 is provided. In some embodiments, the tissue-specific expression of human or humanized CD94 protein is provided. In some embodiments, a non-human mammal expressing human or humanized NKG2A is provided. In some embodiments, the tissue-specific expression of human or humanized NKG2A protein is provided.

In some embodiments, the expression of human or humanized CD94 in a genetically modified animal is controllable, as by the addition of a specific inducer or repressor substance. In some embodiments, the expression of human or humanized NKG2A in a genetically modified animal is controllable, as by the addition of a specific inducer or repressor substance. In some embodiments, the specific inducer is selected from Tet-Off System/Tet-On System, or Tamoxifen System.

Non-human mammals can be any non-human animal known in the art and which can be used in the methods as described herein. Preferred non-human mammals are mammals, (e.g., rodents). In some embodiments, the non-human mammal is a mouse.

Genetic, molecular and behavioral analyses for the non-human mammals described above can performed. The present disclosure also relates to the progeny produced by the non-human mammal provided by the present disclosure mated with the same or other genotypes.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cell can be obtained from the cell culture established using the same constructs and the standard cell transfection techniques. The integration of genetic constructs containing DNA sequences encoding human CD94 and/or NKG2A protein can be detected by a variety of methods.

There are many analytical methods that can be used to detect exogenous DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human or humanized CD94 and/or NKG2A protein.

### Vectors

The present disclosure relates to a targeting vector, comprising: a) a DNA fragment homologous to the 5′ end of a region to be altered (5′ arm), which is selected from the CD94 gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the CD94 gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000072.6) the DNA fragment homologous to the 3′ end of the region to be altered (3′ arm) is selected from the nucleotide sequences that have at least 90% homology to the NCBI accession number NC_000072.6.

In some embodiments, a) the DNA fragment homologous to the 5′ end of a region to be altered (5′ arm) is selected from the nucleotides from the position 129590135 to the position 129593735 of the NCBI accession number NC_000072.6; c) the DNA fragment homologous to the 3′ end of the region to be altered (3′ arm) is selected from the nucleotides from the position 129599166 to the position 129603653 of the NCBI accession number NC_000072.6.

In some embodiments, a) the DNA fragment homologous to the 5′ end of a region to be altered (5′ arm) is selected from the nucleotides from the position 129682351 to the position 129678300 of the NCBI accession number NC_000072.6; c) the DNA fragment homologous to the 3′ end of the region to be altered (3′ arm) is selected from the nucleotides from the position 129674508 to the position 129669878 of the NCBI accession number NC_000072.6.

In some embodiments, the length of the selected genomic nucleotide sequence in the targeting vector can be about 1 kB, about 1.5 kb, about 2 kb, about 2.5 kb, about 3 kb, about 3.5 kb, about 4 kb, about 4.5 kb, about 5 kb, about 5.5 kb, or about 6 kb.

In some embodiments, the region to be altered is exon 1, exon 2, exon 3, exon 4, exon 5, and/or exon 6 of an endogenous CD94 gene (e.g., a sequence starting within exon 3 and ending within exon 6 of mouse CD94 gene).

In some embodiments, the region to be altered is exon 1, exon 2, exon 3, exon 4, exon 5, exon 6, and/or exon 7 of an endogenous NKG2A gene (e.g., a sequence starting within exon 3 and ending within exon 6 of mouse NKG2A gene).

The targeting vector can further include a selected gene marker.

In some embodiments, the sequence of the 5′ arm is shown in SEQ ID NO: 3 or at least 90% identical to SEQ ID NO: 3; and the sequence of the 3′ arm is shown in SEQ ID NO: 4 or at least 90% identical to SEQ ID NO: 4. In some embodiments, the sequence of the selected genomic nucleotide sequence is shown in SEQ ID NO: 5.

In some embodiments, the sequence of the 5′ arm is shown in SEQ ID NO: 30 or at least 90% identical to SEQ ID NO: 30; and the sequence of the 3′ arm is shown in SEQ ID NO: 31 or at least 90% identical to SEQ ID NO: 31. In some embodiments, the sequence of the selected genomic nucleotide sequence is shown in SEQ ID NO: 32.

In some embodiments, the sequence is derived from human (e.g., 10309634-10314790 of NC_000012.12). For example, the target region in the targeting vector is a part or entirety of the nucleotide sequence of a human CD94, preferably a sequence starting within exon 4 and ending within exon 7 of the human CD94. In some embodiments, the nucleotide sequence of the humanized CD94 encodes the entire or the part of human CD94 protein with the NCBI accession number NP_001337991.1 (SEQ ID NO: 2).

In some embodiments, the sequence is derived from human (e.g., 10450487-10446554 of NC_000012.12). For example, the target region in the targeting vector is a part or entirety of the nucleotide sequence of a human NKG2A, preferably a sequence starting within exon 4 and ending within exon 8 of the human NKG2A. In some embodiments, the nucleotide sequence of the humanized CD94 encodes the entire or the part of human NKG2A protein with the NCBI accession number NP_998823.1 (SEQ ID NO: 29).

The disclosure also relates to a cell comprising the targeting vectors as described above.

In addition, the present disclosure further relates to a non-human mammalian cell, having any one of the foregoing targeting vectors, and one or more in vitro transcripts of the construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the non-human mammalian cell is a mouse cell. In some embodiments, the cell is a fertilized egg cell.

The disclosure also provides vectors for constructing a humanized animal model or a knock-out model. In some embodiments, the vectors comprise sgRNA sequence, wherein the sgRNA sequence target CD94 or NKG2A gene, and the sgRNA is unique on the target sequence of the gene to be altered. In some embodiments, the sgRNA meets the sequence arrangement rule of 5′—NNN (20) -NGG3′ or 5′—CCN—N (20)-3′.

In some embodiments, the targeting site of the sgRNA in the mouse CD94 gene is located on the exon 1, exon 2, exon 3, exon 4, exon 5, exon 6, intron 1, intron 2, intron 3, intron 4, intron 5, upstream of exon 1, or downstream of exon 6 of the mouse CD94 gene. In some embodiments, the 5′ targeting site is located on exon 1 of the mouse CD94 gene. In some embodiments, the 5′ targeting site is located on exon 3 of the mouse CD94 gene. In some embodiments, the 3′ targeting site is located on exon 6 of the mouse CD94 gene.

In some embodiments, the targeting site of the sgRNA in the mouse NKG2A gene is located on the exon 1, exon 2, exon 3, exon 4, exon 5, exon 6, exon 7, intron 1, intron 2, intron 3, intron 4, intron 5, intron 6, upstream of exon 1, or downstream of exon 7 of the mouse NKG2A gene. In some embodiments, the 5′ targeting site is located on exon 1 of the mouse NKG2A gene. In some embodiments, the 5′ targeting site is located on exon 2 of the mouse NKG2A gene. In some embodiments, the 3′ targeting site is located on exon 6 of the mouse NKG2A gene. In some embodiments, the 3′ targeting site is located on exon 7 of the mouse NKG2A gene.

In some embodiments, the 5′ targeting site sequences of the sgRNA are shown as SEQ ID NOs: 44-51, and the sgRNA recognizes the 5′ targeting site. In some embodiments, the 3′ targeting sequences for the sgRNA are shown as SEQ ID NOs: 52-58 and the sgRNA recognizes the 3′ targeting site. In some embodiments, the 5′ targeting sequence is SEQ ID NO: 44 and the 3′ targeting sequence is SEQ ID NO: 58. Thus, the disclosure provides sgRNA sequences for constructing a genetic modified animal model.

In some embodiments, the disclosure provides DNA sequences encoding the sgRNAs. In some embodiments, the disclosure relates to a plasmid construct (e.g., pT7-sgRNA) including the sgRNA sequence, and/or a cell including the construct.

### Methods of Making Genetically Modified Animals

Genetically modified animals can be made by several techniques that are known in the art, including, e.g., nonhomologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides replacing in at least one cell of the animal, at an endogenous CD94 gene locus, a sequence encoding a region of an endogenous CD94 with a sequence encoding a corresponding region of human or chimeric CD94. Thus, in some embodiments, the disclosure provides replacing in at least one cell of the animal, at an endogenous NKG2A gene locus, a sequence encoding a region of an endogenous NKG2A with a sequence encoding a corresponding region of human or chimeric NKG2A. In some embodiments, the replacement occurs in a germ cell, a somatic cell, a blastocyst, or a fibroblast, etc. The nucleus of a somatic cell or the fibroblast can be inserted into an enucleated oocyte.

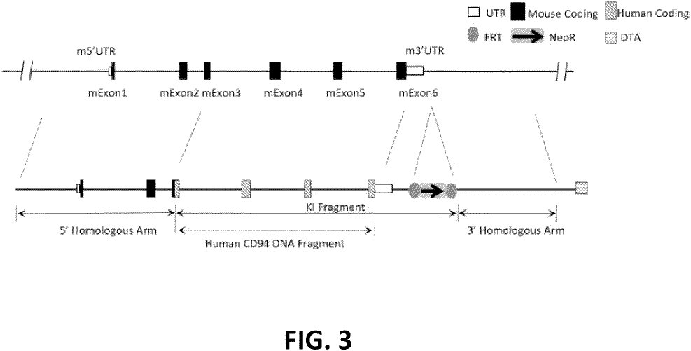

FIG. 3 shows a humanization strategy for a mouse CD94 locus. In FIG. 3, the targeting strategy involves a vector comprising the 5′ end homologous arm, human CD94 gene fragment, 3′ homologous arm. The process can involve replacing endogenous CD94 sequence with human sequence by homologous recombination. In some embodiments, the cleavage at the upstream and the downstream of the target site (e.g., by zinc finger nucleases, TALEN or CRISPR) can result in DNA double strands break, and the homologous recombination is used to replace endogenous CD94 sequence with human CD94 sequence.

Thus, in some embodiments, the methods for making a genetically modified, humanized animal, can include the step of replacing at an endogenous CD94 locus (or site), a sequence encoding a region of endogenous CD94 with a sequence encoding a corresponding region of human CD94. The replaced sequence can include a region (e.g., a part or the entire region) of exon 3, exon 4, exon 5, and/or exon 6 of an endogenous CD94 gene. In some embodiments, the region is located within the extracellular region of CD94. In some embodiments, the sequence encoding a corresponding region of human CD94 includes a region of exon 4, exons 5-6, and a region of exon 7 of a human CD94 gene (e.g., a sequence encoding amino acids 37-179 of SEQ ID NO: 2). In some embodiments, the replaced sequence includes a region of exon 3, exons 4-5, and a region of exon 6 of mouse CD94 gene (e.g., a sequence encoding amino acids 37-179 of SEQ ID NO: 1).

In some embodiments, the region is located within the extracellular region of CD94. In some embodiments, the sequence encoding a region of endogenous CD94 includes a portion of exon 3 and a portion of exon 4 of mouse CD94.

In some embodiments, the methods of modifying a CD94 locus of a mouse to express a chimeric human/mouse CD94 peptide can include the steps of replacing at the endogenous mouse CD94 locus a nucleotide sequence encoding a mouse CD94 with a nucleotide sequence encoding a human CD94, thereby generating a sequence encoding a chimeric human/mouse CD94.

In some embodiments, provided herein is a genetically-modified non-human animal whose genome comprises a sequence that is at least 70%, 75%, 80%, 85%, 90%, 95%, 99%, or 100% identical to SEQ ID NO: 1, 2, or 8.

In some embodiments, the methods described herein include insertion or replacement at a non-human animal CD94 gene locus, using a nucleotide sequence of humanized CD94 gene. In some embodiments, the insertion or replacement site is after the endogenous regulatory element of the CD94 gene. In some embodiments, the insertion includes disrupting the coding frame of the endogenous CD94 gene of the non-human animal or disrupting the coding frame of the endogenous CD94 gene after the insertion site first, followed by inserting the nucleotide sequence. Alternatively, the insertion step can cause frameshift mutations to the endogenous CD94 gene and also insert human sequences at the same time. In some embodiments, auxiliary sequences (e.g., stop codons or sequences containing termination functions, etc.) or other sequences (e.g., flipping sequences, or knockout sequences) can be added to the nucleotide sequence to make the endogenous CD94 protein of the non-human animal (encoded by a sequence after the insertion site) not express normally.

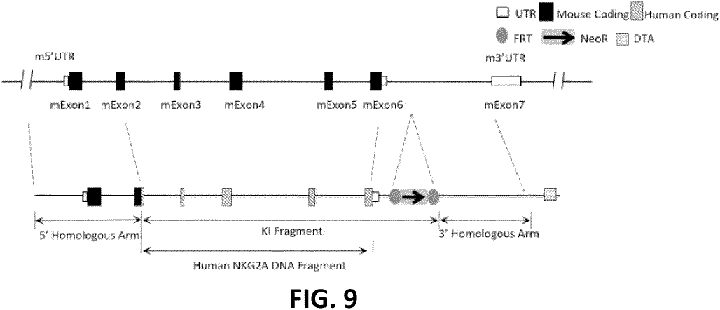

FIG. 9 shows a humanization strategy for a mouse NKG2A locus. In FIG. 9, the targeting strategy involves a vector comprising the 5′ end homologous arm, human NKG2A gene fragment, 3′ homologous arm. The process can involve replacing endogenous NKG2A sequence with human sequence by homologous recombination. In some embodiments, the cleavage at the upstream and the downstream of the target site (e.g., by zinc finger nucleases, TALEN or CRISPR) can result in DNA double strands break, and the homologous recombination is used to replace endogenous NKG2A sequence with human NKG2A sequence.

Thus, in some embodiments, the methods for making a genetically modified, humanized animal, can include the step of replacing at an endogenous NKG2A locus (or site), a sequence encoding a region of endogenous NKG2A with a sequence encoding a corresponding region of human NKG2A. The replaced sequence can include a region (e.g., a part or the entire region) of exon 2, exon 3, exon 4, exon 5, and/or exon 6 of an endogenous NKG2A gene. In some embodiments, the region is located within the extracellular region of NKG2A. In some embodiments, the sequence encoding a corresponding region of human NKG2A includes a region of exon 4, exons 5-7, and a region of exon 8 of a human NKG2A gene (e.g., a sequence encoding amino acids 94-233 of SEQ ID NO: 29). In some embodiments, the replaced sequence includes a region of exon 2, exons 3-5, and a region of exon 6 of mouse NKG2A gene (e.g., a sequence encoding amino acids 96-244 of SEQ ID NO: 28).

In some embodiments, the methods of modifying a NKG2A locus of a mouse to express a chimeric human/mouse NKG2A peptide can include the steps of replacing at the endogenous mouse NKG2A locus a nucleotide sequence encoding a mouse NKG2A with a nucleotide sequence encoding a human NKG2A, thereby generating a sequence encoding a chimeric human/mouse NKG2A.

In some embodiments, provided herein is a genetically-modified non-human animal whose genome comprises a sequence that is at least 70%, 75%, 80%, 85%, 90%, 95%, 99%, or 100% identical to SEQ ID NO: 28, 29, or 35.

In some embodiments, the methods described herein include insertion or replacement at a non-human animal NKG2A gene locus, using a nucleotide sequence of humanized NKG2A gene. In some embodiments, the insertion or replacement site is after the endogenous regulatory element of the NKG2A gene. In some embodiments, the insertion includes disrupting the coding frame of the endogenous NKG2A gene of the non-human animal or disrupting the coding frame of the endogenous NKG2A gene after the insertion site first, followed by inserting the nucleotide sequence. Alternatively, the insertion step can cause frameshift mutations to the endogenous NKG2A gene and also insert human sequences at the same time. In some embodiments, auxiliary sequences (e.g., stop codons or sequences containing termination functions, etc.) or other sequences (e.g., flipping sequences, or knockout sequences) can be added to the nucleotide sequence to make the endogenous NKG2A protein of the non-human animal (encoded by a sequence after the insertion site) not express normally.

In some embodiments, the nucleotide sequences as described herein do not overlap with each other (e.g., the 5′ homologous arm, the “KI fragment”, and/or the 3′ homologous arm do not overlap). In some embodiments, the amino acid sequences as described herein do not overlap with each other.

The present disclosure further provides a method for establishing a CD94 and/or NKG2A gene humanized animal model, involving the following steps:
(a) providing the cell (e.g. a fertilized egg cell) based on the methods described herein;
(b) culturing the cell in a liquid culture medium;
(c) transplanting the cultured cell to the fallopian tube or uterus of the recipient female non-human mammal, allowing the cell to develop in the uterus of the female non-human mammal;
(d) identifying the germline transmission in the offspring genetically modified humanized non-human mammal of the pregnant female in step (c).

In some embodiments, the non-human mammal in the foregoing method is a mouse (e.g., a C57BL/6 mouse).

In some embodiments, the non-human mammal in step (c) is a female with pseudo pregnancy (or false pregnancy).

In some embodiments, the embryonic stem cells for the methods described above are C57BL/6 embryonic stem cells. Other embryonic stem cells that can also be used in the methods as described herein include, but are not limited to, FVB/N embryonic stem cells, BALB/c embryonic stem cells, DBA/1 embryonic stem cells and DBA/2 embryonic stem cells.

Embryonic stem cells can come from any non-human animal, e.g., any non-human animal as described herein. In some embodiments, the embryonic stem cells are derived from rodents. The genetic construct can be introduced into an embryonic stem cell by microinjection of DNA. For example, by way of culturing an embryonic stem cell after microinjection, a cultured embryonic stem cell can be transferred to a false pregnant non-human animal, which then gives birth of a non-human mammal, so as to generate the non-human mammal mentioned in the methods described above.

### Methods of Using Genetically Modified Animals

Replacement of non-human genes in a non-human animal with homologous or orthologous human genes or human sequences, at the endogenous non-human locus and under control of endogenous promoters and/or regulatory elements, can result in a non-human animal with qualities and characteristics that may be substantially different from a typical knockout-plus-transgene animal. In the typical knockout-plus-transgene animal, an endogenous locus is removed or damaged and a fully human transgene is inserted into the animal’s genome and presumably integrates at random into the genome. Typically, the location of the integrated transgene is unknown; expression of the human protein is measured by transcription of the human gene and/or protein assay and/or functional assay. Inclusion in the human transgene of upstream and/or downstream human sequences are apparently presumed to be sufficient to provide suitable support for expression and/or regulation of the transgene.

In some cases, the transgene with human regulatory elements expresses in a manner that is unphysiological or otherwise unsatisfactory, and can be actually detrimental to the animal. The disclosure demonstrates that a replacement with human sequence at an endogenous locus under control of endogenous regulatory elements provides a physiologically appropriate expression pattern and level that results in a useful humanized animal whose physiology with respect to the replaced gene are meaningful and appropriate in the context of the humanized animal’s physiology.

Genetically modified animals that express human or humanized CD94 and/or NKG2A protein, e.g., in a physiologically appropriate manner, provide a variety of uses that include, but are not limited to, developing therapeutics for human diseases and disorders, and assessing the toxicity and/or the efficacy of these human therapeutics in the animal models.

In various aspects, genetically modified animals are provided that express human or humanized CD94, which are useful for testing agents that can decrease or block the interaction between CD94 and NKG2 molecules (e.g., NKG2A) or the interaction between CD94 and anti-human CD94 antibodies, testing whether an agent can increase or decrease the immune response, and/or determining whether an agent is an CD94 agonist or antagonist. In various aspects, genetically modified animals are provided that express human or humanized NKG2A, which are useful for testing agents that can decrease or block the interaction between NKG2A and CD94, or the interaction between NKG2A and anti-human NKG2A antibodies, testing whether an agent can increase or decrease the immune response, and/or determining whether an agent is an NKG2A agonist or antagonist. The genetically modified animals can be, e.g., an animal model of a human disease, e.g., the disease is induced genetically (a knock-in or knockout). In various embodiments, the genetically modified non-human animals further comprise an impaired immune system, e.g., a non-human animal genetically modified to sustain or maintain a human xenograft, e.g., a human solid tumor or a blood cell tumor (e.g., a lymphocyte tumor, e.g., a B or T cell tumor).

In some embodiments, the genetically modified animals can be used for determining effectiveness of an anti-CD94 antibody for the treatment of cancer. The methods involve administering the anti-CD94 antibody (e.g., anti-human CD94 antibody) to the animal as described herein, wherein the animal has a tumor; and determining the inhibitory effects of the anti-CD94 antibody to the tumor. In some embodiments, the genetically modified animals can be used for determining effectiveness of an anti-NKG2A antibody for the treatment of cancer. The methods involve administering the anti-NKG2A antibody (e.g., anti-human NKG2A antibody) to the animal as described herein, wherein the animal has a tumor; and determining the inhibitory effects of the anti-NKG2A antibody to the tumor. The inhibitory effects that can be determined include, e.g., a decrease of tumor size or tumor volume, a decrease of tumor growth, a reduction of the increase rate of tumor volume in a subject (e.g., as compared to the rate of increase in tumor volume in the same subject prior to treatment or in another subject without such treatment), a decrease in the risk of developing a metastasis or the risk of developing one or more additional metastasis, an increase of survival rate, and an increase of life expectancy, etc. The tumor volume in a subject can be determined by various methods, e.g., as determined by direct measurement, MRI or CT.

In some embodiments, the tumor comprises one or more cancer cells (e.g., human or mouse cancer cells) that are injected into the animal. In some embodiments, the anti-CD94 antibody prevents MHC class I molecules from binding to a CD94/NKG2 receptor. In some embodiments, the anti-CD94 antibody does not prevent MHC class I molecules from binding to a CD94/NKG2 receptor.

In some embodiments, the tumor comprises one or more cancer cells (e.g., human or mouse cancer cells) that are injected into the animal. In some embodiments, the anti-NKG2A antibody prevents MHC class I molecules from binding to a CD94/NKG2 receptor. In some embodiments, the anti-NKG2A antibody does not prevent MHC class I molecules from binding to a CD94/NKG2 receptor. Exemplary anti-NKG2A antibodies include monalizumab (See PCT Publication No.: WO2016041945A1).

In some embodiments, the genetically modified animals can be used for determining whether an anti-CD94 antibody is a CD94 agonist or antagonist. In some embodiments, the methods as described herein are also designed to determine the effects of the agent (e.g., anti-CD94 antibodies) on CD94. In some embodiments, the genetically modified animals can be used for determining whether an anti-NKG2A antibody is a NKG2A agonist or antagonist. In some embodiments, the methods as described herein are also designed to determine the effects of the agent (e.g., anti-NKG2A antibodies) on NKG2A. The effects can include, whether the agent can stimulate immune cells or inhibit immune cells (e.g., NK cells or CD8+ T lymphocytes), whether the agent can increase or decrease the production of cytokines, whether the agent can activate or deactivate immune cells (e.g., NK cells or CD8+ T lymphocytes), whether the agent can upregulate the immune response or downregulate immune response, and/or whether the agent can induce complement mediated cytotoxicity (CMC) or antibody dependent cellular cytoxicity (ADCC). In some embodiments, the genetically modified animals can be used for determining the effective dosage of a therapeutic agent for treating a disease in the subject, e.g., cancer, or autoimmune diseases.

The inhibitory effects on tumors can also be determined by methods known in the art, e.g., measuring the tumor volume in the animal, and/or determining tumor (volume) inhibition rate (TGI_{TV}). The tumor growth inhibition rate can be calculated using the formula TGI_{TV} (%) = (1 -TVt/TVc) x 100, where TVt and TVc are the mean tumor volume (or weight) of treated and control groups.

In some embodiments, the anti-CD94 antibody or anti-NKG2A antibody is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the cancer types as described herein include, but not limited to, lymphoma, non-small cell lung cancer (NSCLC), leukemia, ovarian cancer, nasopharyngeal cancer, breast cancer, endometrial cancer, colon cancer, rectal cancer, stomach cancer, bladder cancer, lung cancer, bronchial cancer, bone cancer, prostate cancer, pancreatic cancer, liver and bile duct cancer, esophageal cancer, kidney cancer, thyroid cancer, head and neck cancer, testicular cancer, glioblastoma, astrocytoma, melanoma, myelodysplastic syndrome, and sarcoma. In some embodiments, the leukemia is selected from acute lymphocytic (lymphoblastic) leukemia, acute myeloid leukemia, myeloid leukemia, chronic lymphocytic leukemia, multiple myeloma, plasma cell leukemia, and chronic myelogenous leukemia. In some embodiments, the lymphoma is selected from Hodgkin’s lymphoma and non-Hodgkin’s lymphoma, including B-cell lymphoma, diffuse large B-cell lymphoma, follicular lymphoma, mantle cell lymphoma, marginal zone B-cell lymphoma, T cell lymphoma, and Waldenstrom macroglobulinemia. In some embodiments, the sarcoma is selected from osteosarcoma, Ewing sarcoma, leiomyosarcoma, synovial sarcoma, soft tissue sarcoma, angiosarcoma, liposarcoma, fibrosarcoma, rhabdomyosarcoma, and chondrosarcoma. In some embodiments, the cancer types as described herein also include gynecologic cancer, ovarian cancer, uterine cancer, vaginal cancer, cervical cancer, vulvar cancer, head and neck cancer (head and neck squamous cell carcinoma (HNSCC)), non-small cell lung cancer (NSCLC), hematological cancer, solid tumor, breast cancer, chronic lymphocytic leukemia, squamous cell carcinoma of the oral cavity, colorectal cancer, liver cancer, glioblastoma, Hodgkin lymphoma, esophagus cancer, gastric cancer, pancreas cancer, renal cancer, lung cancer, or melanoma. In some embodiments, the cancer type is head and neck squamous cell carcinoma (HNSCC).

In some embodiments, the antibody is designed for treating various immune disorder or immune-related diseases (e.g., rheumatoid arthritis, psoriasis, allergic rhinitis, sinusitis, asthma, atopic dermatitis, chronic obstructive pulmonary disease (COPD), chronic bronchitis, emphysema, eczema, osteoarthritis, rheumatoid arthritis, systemic lupus erythematosus, polymyalgia rheumatica, autoimmune hemolytic anemia, systemic vasculitis, pernicious anemia, inflammatory bowel disease, ulcerative colitis, Crohn’s disease, or multiple sclerosis). Thus, the methods as described herein can be used to determine the effectiveness of an anti-CD94 or anti-NKG2A antibody in inhibiting immune response.

In some embodiments, the immune disorder or immune-related diseases described here include allergy, asthma, myocarditis, nephritis, hepatitis, systemic lupus erythematosus, rheumatoid arthritis, scleroderma, hyperthyroidism, primary thrombocytopenic purpura, autoimmune hemolytic anemia, ulcerative colitis, self-immune liver disease, diabetes, pain, or neurological disorders.

In some embodiments, the antibody is designed for reducing inflammation (e.g., inflammatory bowel disease, chronic inflammation, asthmatic inflammation, periodontitis, or wound healing). Thus, the methods as described herein can be used to determine the effectiveness of an antibody for reducing inflammation. In some embodiments, the inflammation described herein includes degenerative inflammation, exudative inflammation, serous inflammation, fibrinitis, suppurative inflammation, hemorrhagic inflammation, necrotitis, catarrhal inflammation, proliferative inflammation, specific inflammation, tuberculosis, syphilis, leprosy, or lymphogranuloma.

In some embodiments, the antibody is designed for treating viral infection, e.g., HIV-induced infection, EBV-induced infection, or HPV-induced infection.

The present disclosure also provides methods of determining toxicity of an antibody (e.g., anti-CD94 antibody or anti-NKG2A antibody). The methods involve administering the antibody to the animal as described herein. The animal is then evaluated for its weight change, red blood cell count, hematocrit, and/or hemoglobin. In some embodiments, the antibody can decrease the red blood cells (RBC), hematocrit, or hemoglobin by more than 20%, 30%, 40%, or 50%. In some embodiments, the animals can have a weight that is at least 5%, 10%, 20%, 30%, or 40% smaller than the weight of the control group (e.g., average weight of the animals that are not treated with the antibody).

The present disclosure also relates to the use of the animal model generated through the methods as described herein in the development of a product related to an immunization processes of human cells, the manufacturing of a human antibody, or the model system for a research in pharmacology, immunology, microbiology and medicine.

In some embodiments, the disclosure provides the use of the animal model generated through the methods as described herein in the production and utilization of an animal experimental disease model of an immunization processes involving human cells, the study on a pathogen, or the development of a new diagnostic strategy and/or a therapeutic strategy.

The disclosure also relates to the use of the animal model generated through the methods as described herein in the screening, verifying, evaluating or studying the CD94 gene function, human CD94 antibodies, drugs for human CD94 targeting sites, the drugs or efficacies for human CD94 targeting sites, the drugs for immune-related diseases and antitumor drugs. The disclosure also relates to the use of the animal model generated through the methods as described herein in the screening, verifying, evaluating or studying the NKG2A gene function, human NKG2A antibodies, drugs for human NKG2A targeting sites, the drugs or efficacies for human NKG2A targeting sites, the drugs for immune-related diseases and antitumor drugs.

In some embodiments, the disclosure provides a method to verify in vivo efficacy of TCR-T, CAR-T, and/or other immunotherapies (e.g., T-cell adoptive transfer therapies). For example, the methods include transplanting human tumor cells into the animal described herein, and applying human CAR-T to the animal with human tumor cells. Effectiveness of the CAR-T therapy can be determined and evaluated. In some embodiments, the animal is selected from the CD94 gene and/or NKG2A gene humanized non-human animals prepared by the methods described herein, the CD94 gene and/or NKG2A gene humanized non-human animals described herein, the double- or multi-humanized non-human animal generated by the methods described herein (or progeny thereof), a non-human animal expressing the human or humanized CD94 protein and/or NKG2A protein, or the tumor-bearing or inflammatory animal models described herein. In some embodiments, the TCR-T, CAR-T, and/or other immunotherapies can treat the CD94-associated and/or NKG2A-associated diseases described herein. In some embodiments, the TCR-T, CAR-T, and/or other immunotherapies provides an evaluation method for treating the CD94-associated and/or NKG2A-associated diseases described herein.

### Genetically Modified Animal Model With Two or More Human or Chimeric Genes

The present disclosure further relates to methods for generating genetically modified animal model with two or more human or chimeric genes. The animal can comprise a human or chimeric CD94 and/or NKG2A gene and a sequence encoding an additional human or chimeric protein.

In some embodiments, the additional human or chimeric protein can be programmed cell death protein 1 (PD-1), B7 Homolog 3 (B7-H3), V-set domain-containing T-cell activation inhibitor 1 (B7-H4), Interleukin-2 (IL-2), Interleukin-23 subunit alpha (IL23A), C-C Motif Chemokine Receptor 2 (CCR2), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α (SIRPα) or TNF Receptor Superfamily Member 4 (TNFRSF4 or OX40).

The methods of generating genetically modified animal model with two or more human or chimeric genes (e.g., humanized genes) can include the following steps:
(a) using the methods of introducing human CD94 gene or chimeric CD94 gene as described herein, or the methods of introducing human NKG2A gene or chimeric NKG2A gene as described herein, to obtain a genetically modified non-human animal;
(b) breeding the genetically modified non-human animal with another genetically modified non-human animal, and then screening the progeny to obtain a genetically modified non-human animal with two or more human or chimeric genes.

In some embodiments, in step (b) of the method, the genetically modified animal can be mated with a genetically modified non-human animal with human or chimeric PD-1, B7-H3, B7-H4, IL-2, IL23A, CCR2, CTLA-4, LAG-3, BTLA, PD-L1, CD27, CD28, CD47, CD137, CD154, TIGIT, TIM-3, GITR, SIRPα, or OX40. Some of these genetically modified non-human animal are described, e.g., in PCT/CN2017/090320, PCT/CN2017/099577, PCT/CN2017/099575, PCT/CN2017/099576, PCT/CN2017/099574, PCT/CN2017/106024, PCT/CN2017/110494, PCT/CN2017/110435, PCT/CN2017/120388, PCT/CN2018/081628, PCT/CN2018/081629; each of which is incorporated herein by reference in its entirety.

In some embodiments, the CD94 gene and/or NKG2A gene humanization is directly performed on a genetically modified animal having a human or chimeric PD-1, B7-H3, B7-H4, IL-2, IL23A, CCR2, CTLA-4, BTLA, PD-L1, CD27, CD28, CD47, CD137, CD154, TIGIT, TIM-3, GITR, SIRPα, or OX40 gene.

As these proteins may involve different mechanisms, a combination therapy that targets two or more of these proteins thereof may be a more effective treatment. In fact, many related clinical trials are in progress and have shown a good effect. The genetically modified animal model with two or more human or humanized genes can be used for determining effectiveness of a combination therapy that targets two or more of these proteins, e.g., an anti-CD94 and/or anti-NKG2A antibody and an additional therapeutic agent (e.g., an anti-PD-1 antibody) for the treatment of cancer. The methods include administering the anti-CD94 and/or anti-NKG2A antibody and the additional therapeutic agent (e.g., an anti-PD-1 antibody) to the animal, wherein the animal has a tumor; and determining the inhibitory effects of the combined treatment to the tumor. In some embodiments, the additional therapeutic agent is an antibody that specifically binds to PD-1, B7-H3, B7-H4, IL-2, IL23A, CCR2, CTLA-4, BTLA, PD-L1, CD27, CD28, CD47, CD137, CD154, TIGIT, TIM-3, GITR, SIRPa, or OX40. In some embodiments, the additional therapeutic agent is an anti-CTLA4 antibody (e.g., ipilimumab), an anti-PD-1 antibody (e.g., nivolumab), an anti-PD-L1 antibody, or an anti-EGFR (epidermal growth factor receptor) antibody (e.g., cetuximab).

In some embodiments, the animal further comprises a sequence encoding a human or humanized PD-1, a sequence encoding a human or humanized PD-L1, or a sequence encoding a human or humanized CTLA-4. In some embodiments, the additional therapeutic agent is an anti-PD-1 antibody (e.g., nivolumab, pembrolizumab), an anti-PD-L1 antibody, or an anti-CTLA-4 antibody. In some embodiments, the tumor comprises one or more tumor cells that express CD80, CD86, PD-L1, and/or PD-L2.

In some embodiments, the combination treatment is designed for treating various cancer as described herein, e.g., melanoma, non-small cell lung carcinoma (NSCLC), small cell lung cancer (SCLC), bladder cancer, prostate cancer (e.g., metastatic hormone-refractory prostate cancer), advanced breast cancer, advanced ovarian cancer, and/or advanced refractory solid tumor. In some embodiments, the combination treatment is designed for treating metastatic solid tumors, NSCLC, melanoma, B-cell non-Hodgkin lymphoma, colorectal cancer, and multiple myeloma. In some embodiments, the combination treatment is designed for treating melanoma, carcinomas (e.g., pancreatic carcinoma), mesothelioma, hematological malignancies (e.g., Non-Hodgkin’s lymphoma, lymphoma, chronic lymphocytic leukemia), or solid tumors (e.g., advanced solid tumors). In some embodiments, the cancer types as described herein also include gynecologic cancer, ovarian cancer, uterine cancer, vaginal cancer, cervical cancer, vulvar cancer, head and neck cancer (head and neck squamous cell carcinoma (HNSCC)), non-small cell lung cancer (NSCLC), hematological cancer, solid tumor, breast cancer, chronic lymphocytic leukemia, squamous cell carcinoma of the oral cavity, colorectal cancer, liver cancer, glioblastoma, Hodgkin lymphoma, esophagus cancer, gastric cancer, pancreas cancer, renal cancer, lung cancer, or melanoma. In some embodiments, the cancer type is head and neck squamous cell carcinoma (HNSCC).

In some embodiments, the methods described herein can be used to evaluate the combination treatment with some other methods. The methods of treating a cancer that can be used alone or in combination with methods described herein, include, e.g., treating the subject with chemotherapy, e.g., campothecin, doxorubicin, cisplatin, carboplatin, procarbazine, mechlorethamine, cyclophosphamide, adriamycin, ifosfamide, melphalan, chlorambucil, bisulfan, nitrosurea, dactinomycin, daunorubicin, bleomycin, plicomycin, mitomycin, etoposide, verampil, podophyllotoxin, tamoxifen, taxol, transplatinum, 5-flurouracil, vincristin, vinblastin, and/or methotrexate. Alternatively or in addition, the methods can include performing surgery on the subject to remove at least a portion of the cancer, e.g., to remove a portion of or all of a tumor(s), from the patient.

### EXAMPLES

The invention is further described in the following examples, which do not limit the scope of the invention described in the claims.

### Materials and Methods

The following materials were used in the following examples.

BglII, StuI, DraIII, and EcoRV restriction enzymes were purchased from NEB. Catalog numbers are R0144M, R0187M, R3510L, and R3195M, respectively.

C57BL/6 mice and Flp transgenic mice were purchased from the China Food and Drugs Research Institute National Rodent Experimental Animal Center.

Brilliant Violet 510™ anti-mouse CD45 antibody (mCD4-BV605) was purchased from BioLegend with catalog number 103138.

PE-Cy™7 Mouse Anti-Mouse NK-1.1 (mNK1.1-PE/Cy7) antibody was purchased from BD Biosciences with catalog number 552878 (under brand BD Pharmingen™).

PE anti-mouse CD159a (NKG2AB6) Antibody (mNKG2A-PE) was purchased from BioLegend with catalog number 142803.

Alexa Fluor® 647-conjugated AffiniPure F(ab′)₂ Fragment Goat Anti-Human IgG, Fcy fragment Specific (minimal cross-reaction to Bovine, Mouse, and Rabbit Serum Proteins) was purchased from Jackson Immuno Research, with catalog number 109-606-170.

PE Mouse IgG2b, κ Isotype Ctrl Antibody was purchased from BioLegend with catalog number 400311.

APC anti-mouse CD94 Antibody (mCD94-APC) was purchased from BioLegend with catalog number 105511.

PE anti-human CD94 Antibody (hCD94-PE) was purchased from BioLegend with catalog number 305504.

APC Rat IgG2a, κ Isotype Ctrl Antibody was purchased from BioLegend with catalog number 400512.

APC Mouse IgG2a, κ Isotype Ctrl Antibody was purchased from BioLegend with catalog number 400220.

Alexa Fluor® 647 Mouse IgG1, κ Isotype Ctrl (FC) was purchased from BioLegend with catalog number 400130.

Zombie NIR™ Fixable Viability Kit was purchased from BioLegend with catalog number 423106.

Human IgG4, κ Isotype Control Antibody was purchased from CrownBio with catalog number C0004-3.

FITC Rat Anti-Mouse CD3 antibody was purchased from BioLegend with catalog number 115506.

### Example 1: Mice With Humanized CD94 Gene

In this example, a non-human animal (e.g., a mouse) was modified to include a nucleotide sequence encoding human CD94 protein, and the obtained genetically-modified non-human animal can express a human or humanized CD94 protein in vivo. The mouse CD94 gene (NCBI Gene ID: 16643, Primary source: MGI: 1196275, UniProt ID: O54707) is located at 129588092 to 129598775 of chromosome 6 (NC_000072.6), and the human CD94 gene (NCBI Gene ID: 3824, Primary source: HGNC: 6378, UniProt ID: Q13241) is located at 10238383 to 10329608 of chromosome 12 (NC_000012.12). The mouse CD94 transcript is NM_010654.4, and the corresponding protein sequence NP_034784.1 is set forth in SEQ ID NO: 1. The human CD94 transcript is NM_001351062.1, and the corresponding protein sequence NP_001337991.1 is set forth in SEQ ID NO: 2. Mouse and human CD94 gene loci are shown in FIG. 1A and FIG. 1B, respectively.

Genetically modified non-human animals can be generated by several gene editing techniques that are known in the art, including but not limited to, zinc finger nucleases (ZFN), transcription activator-like effector-based nucleases (TALEN), homing endonucleases (megakable base ribozyme), the clustered regularly interspaced short palindromic repeats (CRISPR)-Cas system, or other molecular biology techniques. In this example, a nucleotide sequence encoding human CD94 protein was introduced into the endogenous mouse CD94 locus, such that the mouse can express a human or humanized CD94 protein. The method can include insertion of a sequence including a human CD94 gene sequence into the mouse endogenous CD94 locus. For example, the inserted sequence can include a human CD94 DNA or cDNA sequence. It is also possible to include an auxiliary sequence (e.g., a stop codon or a sequence having termination functions, etc.) or to use other methods (e.g., flipping or knocking out) to inactivate the mouse endogenous CD94 gene. The strategy of in situ replacement can also be used, e.g., a direct replacement with human CD94 gene sequence (DNA or cDNA sequence) on the mouse endogenous CD94 gene locus. Here, an in situ replacement strategy of CD94 DNA sequence was used to illustrate how to humanize the mouse CD94 gene.

Mouse cells can be modified by various gene-editing techniques, for example, replacement of specific mouse CD94 gene sequences with human CD94 gene sequences at the endogenous mouse CD94 locus. For example, under control of a mouse CD94 regulatory element, a 4731 bp sequence spanning from exon 3 (including a part of exon 3) to exon 6 (including a part of exon 6) of the mouse CD94 gene can be replaced with a corresponding 5157 bp sequence spanning from exon 4 (including a part of exon 4) to exon 7 (including a part of exon 7) of the human CD94 gene, to obtain a humanized mouse CD94 gene locus as shown in FIG. 2, thereby humanizing mouse CD94 gene.

As shown in the schematic diagram of the targeting strategy in FIG. 3, the targeting vector includes an upstream homologous arm, a downstream homologous arm, and a knock-in (KI) fragment including a human CD94 gene sequence. Sequence of the upstream homologous arm (5′ homologous arm, SEQ ID NO: 3; 3601 bp) is identical to nucleotide sequence of 129590135-129593735 of NCBI accession number NC_000072.6, and sequence of the downstream homologous arm (3′ homologous arm, SEQ ID NO: 4; 4488 bp) is identical to nucleotide sequence of 129599166-129603653 of NCBI accession number NC_000072.6. The KI fragment comprises a sequence spanning from exon 4 (including a part of exon 4) to exon 7 (including a part of exon 7) of a human genomic CD94 gene (SEQ ID NO: 5; 5157 bp), which is identical to nucleotide sequence of 10309634-10314790 of NCBI accession number NC_000012.12.

The upstream of the KI fragment containing the human CD94 gene sequence is directly connected to the 5′ homologous arm, and the connection between the downstream of the human CD94 gene sequence and the mouse CD94 gene locus was designed as: 5′-GAAGATAAAAATCGTTATATCTGTAAGCAACAGCTCATTTAAATGTTTCTTAAGGCA AAGGGTATAGACAAGGAAGGTCC -3′ (SEQ ID NO: 6), wherein the last “T” in sequence “TCATT” is the last nucleotide of the human sequence, and the first “T” of the sequence “TAAAT” is the first nucleotide of the mouse sequence. The mRNA sequence of the engineered mouse CD94 after humanization and its encoded protein sequence are shown in SEQ ID NO: 7 and SEQ ID NO: 8, respectively.

The CD94 gene targeting vector also included an antibiotic resistance gene for positive clone screening (neomycin phosphotransferase gene, or Neo), and two Frt recombination sites flanking the antibiotic resistance gene, that formed a Neo cassette. The connection between the upstream of the Neo cassette and the mouse CD94 gene locus was designed as: 5′-AAGTATGGTAACATATCATCTGCGGATGAAGCTTGATATCGAATTCCGAAGTTCCTA TTCTCTAGAAAGTATAGGAACTT -3′ (SEQ ID NO: 9), wherein the last “G” of the sequence “GATG” is the last nucleotide of the mouse sequence, and the first “A” of the sequence “AAGC” is the first nucleotide of the Neo cassette. The connection between the downstream of the Neo cassette with the mouse CD94 sequence was designed as 5′-TATTCTCTAGAAAGTATAGGAACTTCATCAGTCAGGTACATAATGGTGGATCCAGGC CTGATGTGGTTTGATTGGTTCTGTTCCT -3′ (SEQ ID NO: 10), wherein the “T” of the sequence “GCCT” is the last nucleotide of the Neo cassette, and the first “G” of the sequence “GATGT” is the first nucleotide of the mouse sequence. In addition, a coding gene with a negative selectable marker (a gene encoding diphtheria toxin A subunit (DTA)) was also inserted downstream of the 3′ homologous arm of the targeting vector.

The targeting vector used for replacement of a mouse CD94 gene sequence with the corresponding human CD94 gene sequence was constructed, e.g., by restriction enzyme digestion and ligation, or synthesized directly. Mouse and human CD94 DNA were obtained from bacterial artificial chromosome (BAC) clones RP23-208D19 and RP11-282C10, respectively. The constructed targeting vector sequence was preliminarily confirmed by restriction enzyme digestion, and then verified by sequencing. The correct targeting vector was electroporated and transfected into embryonic stem cells of C57BL/6 mice. The positive selectable marker gene was used to screen the cells, and the integration of exogenous genes was confirmed by PCR and Southern Blot. Specifically, positive clones identified by PCR were further confirmed by Southern Blot to screen out correct positive clone cells used for blastocyst injection.

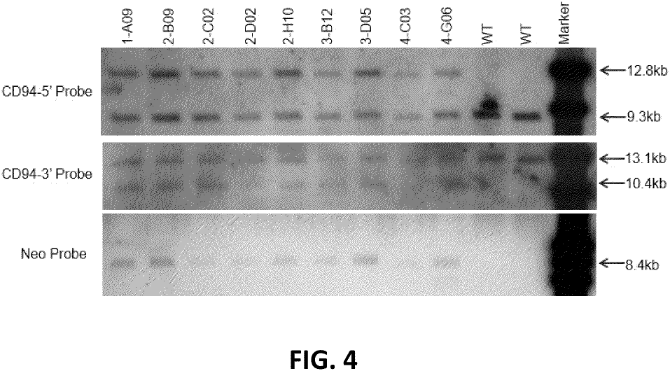

Either the primers CD94-F1 and CD94-R1, or primers CD94-F2 and CD94-R2, were used for PCR amplification, and 9 clones were identified as positive clones with numbers 1-A09, 2-B09, 2-C02, 2-D02, 2-H10, 3-B12, 3-D05, 4-C03, and 4-G06. The positive clones identified by PCR were then verified by Southern Blot. Specifically, genomic DNA of the positive clone cells was digested with BglII, StuI, or DraIII, respectively, and then hybridized with 3 corresponding probes. As shown in FIG. 4, the results indicate that 6 clones (1-A09, 2-B09, 2-C02, 2-H10, 3-B12, and 3-D05) were positive heterozygous clones and no random insertions were detected.

TABLE 5

PCR primers and target band sizes

| | | | |
|---|---|---|---|
| Primer Name | | Sequence (5′-3′) | Fragment size |
| CD94 | F1 | TTCCTGGCAAGAAATGATACTCCA (SEQ ID NO: 11) | 3812 bp |
| | R1 | ATGCCTCTCTGTGTGAAAAACAAG (SEQ ID NO: 12) | |
| CD94 | F2 | CAGGACATAGCGTTGGCTAC (SEQ ID NO: 13) | 5207 bp |
| | R2 | GGAAGAGCCATCACTGTTAAGGT (SEQ ID NO: 14) | |

The following probes were used in Southern Blot assays:
CD94-5′ probe:
F: 5′- ACAAGCCAAACACTAAATTGGCAT -3′ (SEQ ID NO: 15),
R: 5′- GTGGGCCAAGTAGACACTTCCT -3′ (SEQ ID NO: 16);
CD94-3′ probe:
F: 5′- CACAACATTAAGTTTTCCCTCTAGT -3′ (SEQ ID NO: 17),
R: 5′- GATAATCCAGTACTGCCTTGATAGT -3′ (SEQ ID NO: 18);
Neo probe:
F: 5′- GGATCGGCCATTGAACAAGATGG -3′ (SEQ ID NO: 19),
R: 5′- CAGAAGAACTCGTCAAGAAGGCG -3′ (SEQ ID NO: 20).

TABLE 6

| | | | |
|---|---|---|---|
| Restriction Enzyme | Probe | Wild-type fragment size (WT size) | Recombinant sequence fragment size (Targeted size) |
| Bglll | CD94-5′Probe | 9.3 kb | 12.8 kb |
| Stul | CD94-3′Probe | 13.1 kb | 10.4 kb |
| Spel | Neo Probe | -- | 8.4 kb |

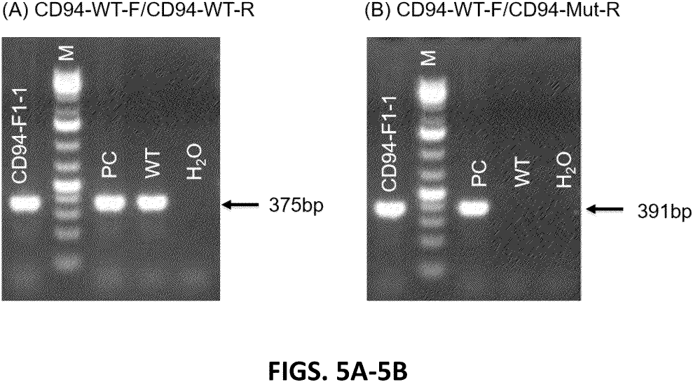

The positive clones that had been screened (black mice) were introduced into isolated blastocysts (white mice), and the resulted chimeric blastocysts were transferred to a culture medium for short-term culture and then transplanted to the fallopian tubes of the recipient mother (white mice) to produce the F0 chimeric mice (black and white). The F2 generation homozygous mice were obtained by backcrossing the F0 generation chimeric mice with wild-type mice to obtain the F1 generation mice, and then breeding the F1 generation heterozygous mice with each other. The positive mice were also bred with the Flp transgenic mice to remove the positive selectable marker gene, and then the humanized CD94 homozygous mice expressing humanized CD94 protein were obtained by breeding with each other. The genotype of the progeny mice can be identified by PCR using primers shown in the table below. The identification results of exemplary F1 generation mice (Neo cassette not removed) are shown in FIGS. 5A-5B, and mice labelled CD94-F1-1 was identified as a positive heterozygous clone. Specifically, primers CD94-WT-F and CD94-WT-R were used to amplify a fragment from endogenous mouse CD94 gene (FIG. 5A); primers CD94-WT-F and CD94-Mut-R were used to amplify a fragment from modified mouse CD94 gene (FIG. 5B), to verify whether the CD94 gene targeting vector was correctly inserted into the mouse genome.

TABLE 7

CD94 gene detection primer sequence

| Primer | | Sequence (5′-3′) | Fragment size (bp) |
|---|---|---|---|
| CD94 | WT-F | TAGTTTCTAGGATCACTCGGTGG (SEQ ID NO: 21) | WT: 375 bp |
| | WT-R | TGCTGTAGAATGAGCTTCTCTGTT (SEQ ID NO: 22) | |
| CD94 | WT-F | TAGTTTCTAGGATCACTCGGTGG (SEQ ID NO: 21) | Mut: 391 bp |
| | Mut-R | ATTCCATCATGCCTCTCTGTGTG (SEQ ID NO: 23) | |

Note: WT stands for wild-type mice; and Mut stands for CD94 gene humanized mice.

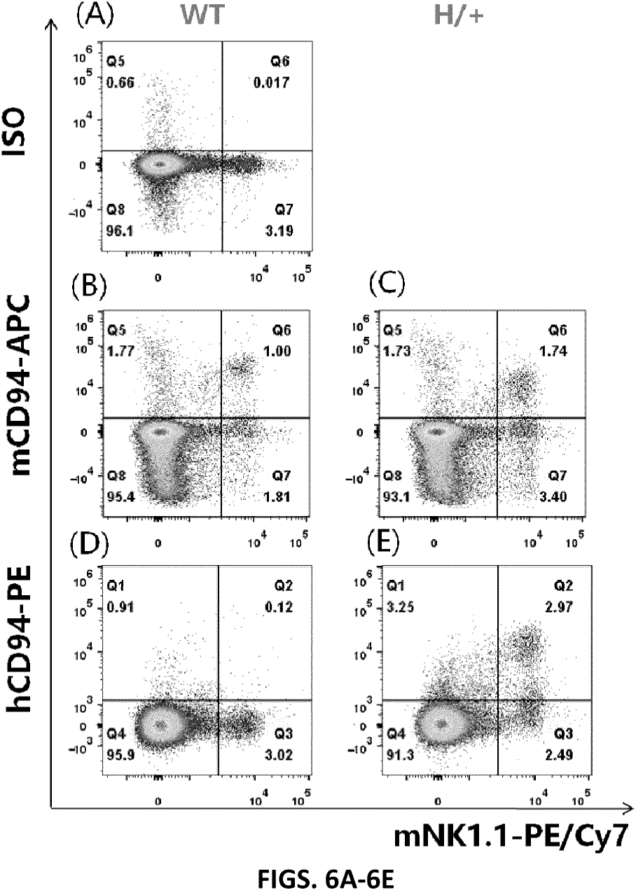

The expression of humanized CD94 protein in CD94 gene humanized heterozygous mice was confirm by flow cytometry. Specifically, one wild-type C57BL/6 mouse (6 weeks old) and one CD94 gene humanized heterozygous mouse (approximately 6-12 weeks old) were selected and the mouse spleens were isolated. Dead spleen cells were stained with vitality dyes (Zombie NIR™, BioLegend) and eliminated by flow cytometry. The live spleen cells were then stained with one of the following combinations of fluorescent dye-labeled antibodies: (1) mNK1.1-PE/Cy7, FITC Rat Anti-Mouse CD3 antibody, and mCD94-APC (FIGS. 6B and 6C); (2) mNK1.1-PE/Cy7, FITC Rat Anti-Mouse CD3 antibody, and hCD94-PE (FIGS. 6D and 6E); or (3) mNK1.1-PE/Cy7, FITC Rat Anti-Mouse CD3 antibody, and APC Rat IgG2a, κ Isotype Ctrl antibody (FIG. 6A), followed by flow cytometry analysis. The results showed that NK cells expressing humanized CD94 protein were detected by the anti-human CD94 antibody in the spleen of the CD94 gene humanized heterozygous mouse (H/+). However, NK cells expressing human or humanized CD94 protein were not detected in the spleen of the wild-type mouse (WT).

### Example 2: Mice With Humanized NKG2A Gene

In this example, a non-human animal (e.g., a mouse) was modified to include a nucleotide sequence encoding human NKG2A protein, and the obtained genetically-modified non-human animal can express a human or humanized NKG2A protein in vivo. The mouse NKG2A gene (NCBI Gene ID: 16641, Primary source: MGI: 1336161, UniProt ID: Q9Z202) is located at 129666015 to 129682852 of chromosome 6 (NC_000072.6), and the human NKG2A gene (NCBI Gene ID: 3821, Primary source: HGNC: 6374, UniProt ID: P26715) is located at 10441673 to 10454685 of chromosome 12 (NC_000012.12). The mouse NKG2A transcript sequence is NM_001136068.2, and the corresponding protein sequence NP_001129540.1 is set forth in SEQ ID NO: 28. The human NKG2A transcript is NM_213658.2, and the corresponding protein sequence NP_998823.1 is set forth in SEQ ID NO: 29. Mouse and human NKG2A gene loci are shown in FIG. 7A and FIG. 7B, respectively.

Genetically modified non-human animals can be generated by several gene editing techniques that are known in the art, including but not limited to, zinc finger nucleases (ZFN), transcription activator-like effector-based nucleases (TALEN), homing endonucleases (megakable base ribozyme), the clustered regularly interspaced short palindromic repeats (CRISPR)-Cas system, or other molecular biology techniques. In this example, a nucleotide sequence encoding human NKG2A protein was introduced into the endogenous mouse NKG2A locus, such that the mouse can express a human or humanized NKG2A protein. The method can include insertion of a sequence including a human NKG2A gene sequence into the mouse endogenous NKG2A locus. For example, the inserted nucleotide sequence can include a human NKG2A DNA or cDNA sequence. It is also possible to include an auxiliary sequence (e.g., a stop codon or a sequence having termination functions, etc.) or to use other methods (e.g., flipping or knocking out) to make the mouse endogenous NKG2A gene unable to express normally. The strategy of in situ replacement can also be used, e.g., a direct replacement with human NKG2A gene sequence (DNA or cDNA sequence of human NKG2A) on the mouse endogenous NKG2A locus. Here, a in situ replacement strategy of NKG2A DNA sequence was used to illustrate how to humanize the mouse NKG2A gene.

Mouse cells can be modified by various gene-editing techniques, for example, replacement of specific mouse NKG2A gene sequences with human NKG2A gene sequences at the endogenous mouse NKG2A locus. For example, under control of a mouse NKG2A regulatory element, a 3438 bp sequence spanning from exon 2 (including a part of exon 2) to exon 6 (including a part of exon 6) of the mouse NKG2A gene can be replaced with a corresponding 3934 bp sequence spanning from exon 4 (including a part of exon 4) to exon 8 (including a part of exon 8) of the human NKG2A gene, to obtain a humanized mouse NKG2A locus as shown in FIG. 8, thereby humanizing mouse NKG2A gene.

As shown in the schematic diagram of the targeting strategy in FIG. 9, the targeting vector includes an upstream homologous arm, a downstream homologous arm, and a knock-in (KI) fragment including a human NKG2A gene sequence. Sequence of the upstream homologous arm (5′ homologous arm, SEQ ID NO: 30; 4052 bp) is identical to nucleotide sequence of 129682351-129678300 of NCBI accession number NC_000072.6, and sequence of the downstream homologous arm (3′ homologous arm, SEQ ID NO: 31; 4631 bp) is identical to nucleotide sequence of 129674508- 129669878 of NCBI accession number NC_000072.6. The KI fragment comprises a sequence spanning from exon 4 (including a part of exon 4) to exon 8 (including a part of exon 8) of a human genomic NKG2A gene (SEQ ID NO: 32; 3934 bp), which is identical to nucleotide sequence of 10450487-10446554 of NCBI accession number NC_000012.12.

The upstream of the KI fragment containing the human NKG2A gene sequence is directly connected to the 5′ homologous arm, and the connection between the downstream of the human NKG2A gene sequence and the mouse NKG2A gene locus was designed as: 5′-TCAATAATATATCATTGTAAGCATAAGCTTTGAAACACCTGCACTGG-3′ (SEQ ID NO: 33), wherein the last “T” in sequence “GCTT” is the last nucleotide of the human sequence, and the “T” of the sequence “TGAA” is the first nucleotide of the mouse sequence. The mRNA sequence of the engineered mouse NKG2A after humanization and its encoded protein sequence are shown in SEQ ID NO: 34 and SEQ ID NO: 35, respectively.

The targeting vector also included an antibiotic resistance gene for positive clone screening (neomycin phosphotransferase gene, or Neo), and two Frt recombination sites flanking the antibiotic resistance gene, that formed a Neo cassette. The connection between the upstream of the Neo cassette and the mouse NKG2A gene locus was designed as: 5′-ATTGCCAGTTGTATATTGCAACTTCAGCTTCTGTAGTACATTTGGGTCGAATTCCGAA GTTCCTATTCTCTAGAAAGTAT -3′ (SEQ ID NO: 36), wherein the “C” of the sequence “GGTC” is the last nucleotide of the mouse sequence, and the “G” of the sequence “GAAT” is the first nucleotide of the Neo cassette. The connection between the downstream of the Neo cassette with the mouse NKG2A sequence was designed as 5′-AGGAACTTCATCAGTCAGGTACATAATTAGGTGGATCCACCCACTTTTAGTCAATAA GTAATATTATATA -3′ (SEQ ID NO: 37), wherein the last “C” of the sequence “ATCC” is the last nucleotide of the Neo cassette, and the “A” of the sequence “ACCC” is the first nucleotide of the mouse sequence. In addition, a coding gene with a negative selectable marker (a gene encoding diphtheria toxin A subunit (DTA)) was also inserted downstream of the 3′ homologous arm of the targeting vector.

The targeting vector used for replacement of a mouse NKG2A gene sequence with the corresponding human NKG2A gene sequence was constructed, e.g., by restriction enzyme digestion and ligation, or synthesized directly. Mouse and human NKG2A DNA were obtained from bacterial artificial chromosome (BAC) clones RP23-164F1 and RP11-653F19, respectively. The constructed targeting vector sequence was preliminarily confirmed by restriction enzyme digestion, and then verified by sequencing. The correct targeting vector was electroporated and transfected into embryonic stem cells of C57BL/6 mice. The positive selectable marker gene was used to screen the cells, and the integration of exogenous genes was confirmed by PCR and Southern Blot. Specifically, positive clones identified by PCR were further confirmed by Southern Blot to screen out correct positive clone cells used for blastocyst injection.

The positive clones that had been screened (black mice) were introduced into isolated blastocysts (white mice), and the resulted chimeric blastocysts were transferred to a culture medium for short-term culture and then transplanted to the fallopian tubes of the recipient mother (white mice) to produce the F0 chimeric mice (black and white). The F2 generation homozygous mice were obtained by backcrossing the F0 generation chimeric mice with wild-type mice to obtain the F1 generation mice, and then breeding the F1 generation heterozygous mice with each other. The positive mice were also bred with the Flp transgenic mice to remove the positive selectable marker gene, and then the humanized NKG2A homozygous mice expressing humanized NKG2A protein were obtained by breeding with each other. The genotype of the progeny mice can be identified by PCR using primers shown in the table below.

TABLE 8

PCR primers and target band sizes

| | | | |
|---|---|---|---|
| Primer | | Sequence (5′-3′) | Fragment size (bp) |
| NKG2A | F1 | ATGCCAGAGGGGAGTACTAATTGCC (SEQ ID NO: 38) | Mut: 4187 bp |
| | R1 | TGGGTTTTCTGGGTACCACACCATT (SEQ ID NO: 39) | |
| NKG2A | F2 | TTGATCCCTGATGCTAGGGTCCCAG (SEQ ID NO: 40) | Mut: 3339 bp |
| | R2 | CTGACACCTGTTGTTGAATTAGCGA (SEQ ID NO: 41) | |
| NKG2A | F1 | ATGCCAGAGGGGAGTACTAATTGCC (SEQ ID NO: 38) | Mut: 1919 bp |
| | R3 | TGCATCAGAAGCGAATACTTTCAACA (SEQ ID NO: 42) | |
| NKG2A | F3 | AGGGGGACTACTCTGTTGAGTGTTCA | Mut: 1909 bp |
| | | (SEQ ID NO: 43) | |
| | R2 | CTGACACCTGTTGTTGAATTAGCGA (SEQ ID NO: 41) | |

CRISPR/Cas gene editing technology was used to obtain the NKG2A gene humanized mice. The target sequences are important for the targeting specificity of sgRNAs and the efficiency of Cas9-induced cleavage. Specific sgRNA sequences were designed and synthesized that recognize the 5′ end targeting site (sgRNA1-sgRNA8) and 3′ end targeting site (sgRNA9-sgRNA15). The 5′ end targeting sites are located on exon 2, and the 3′ end targeting sites are located on exon 6 of the mouse NKG2A gene. The targeting site sequence of each sgRNA on the NKG2A gene locus is as follows:

TABLE 9

sgRNA targeting site sequences on NKG2A gene

| | | |
|---|---|---|
| sgRNA number | Targeting site sequence (5′-3′) | Sequence SEQ ID NO |
| sgRNA1 | GGCACTTCCAGTCATTAAAGTGG | SEQ ID NO: 44 |
| sgRNA2 | AAAAGCATGCTTACATGGTGTGG | SEQ ID NO: 45 |
| sgRNA3 | CCTTGATAGGAAGTTTTGAAAGG | SEQ ID NO: 46 |
| sgRNA4 | TAAACTAAACTGTCCTTGATAGG | SEQ ID NO: 47 |
| sgRNA5 | CCAGCAATGAGTTTCTCTGGAGG | SEQ ID NO: 48 |
| sgRNA6 | GAAACTCATTGCTGGTACCCTGG | SEQ ID NO: 49 |
| sgRNA7 | GTACCAGCAATGAGTTTCTCTGG | SEQ ID NO: 50 |
| sgRNA8 | CCTCCAGAGAAACTCATTGCTGG | SEQ ID NO: 51 |
| sgRNA9 | TGCTTCTGCATAAAGCCTGCTGG | SEQ ID NO: 52 |
| sgRNA10 | GCTTCTGCATAAAGCCTGCTGGG | SEQ ID NO: 53 |
| sgRNA11 | TGACTTTCATGACTGTCGTTAGG | SEQ ID NO: 54 |
| sgRNA12 | TTCAGCTTCTGTAGTACATTTGG | SEQ ID NO: 55 |
| sgRNA13 | TCAGCTTCTGTAGTACATTTGGG | SEQ ID NO: 56 |
| sgRNA14 | TATTACTTATTGACTAAAAGTGG | SEQ ID NO: 57 |
| sgRNA15 | AATCTCTTTCACTGATCTCATGG | SEQ ID NO: 58 |

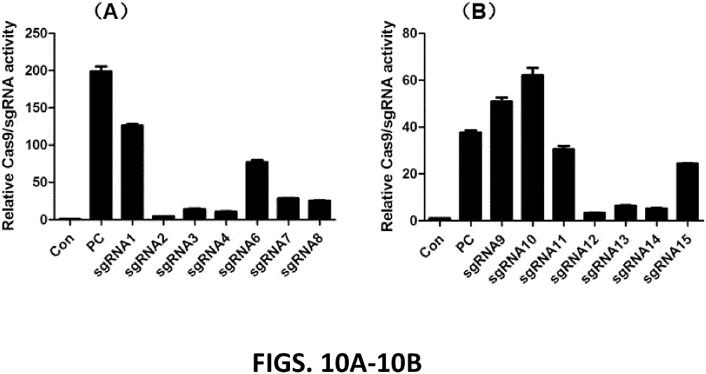

The UCA kit was used to detect the activities of sgRNAs. As shown in FIGS. 10A-10B and the table below, the results showed that the sgRNAs had different activities. sgRNA1 and sgRNA15 were selected for subsequent experiment. Embryonic stem cells of C57BL/6 mice can be transfected with the NKG2A gene targeting vector, together with sgRNA1, sgRNA15, and Cas9 mRNA. The correct positive clones can also be screened.

TABLE 10

sgRNA activity test results

| 5′ end targeting site detection result | | 3′ end targeting site detection result | |
|---|---|---|---|
| Con. | 1.00±0.09 | Con. | 1.00±0.05 |
| PC | 198.56±12.12 | PC | 37.70±1.58 |
| sgRNA-1 | 126.17±3.32 | sgRNA-9 | 50.92±2.98 |
| sgRNA-2 | 4.62±0.17 | sgRNA-10 | 62.10±5.52 |
| sgRNA-3 | 14.21±1.53 | sgRNA-11 | 30.50±2.57 |
| sgRNA-4 | 10.94±0.68 | sgRNA-12 | 3.34±0.34 |
| sgRNA-5 | NA | sgRNA-13 | 6.38±0.46 |
| sgRNA-6 | 76.99±5.54 | sgRNA-14 | 5.24±0.31 |
| sgRNA-7 | 28.49±0.68 | sgRNA-15 | 24.36±0.24 |
| sgRNA-8 | 25.13±1.22 | / | / |

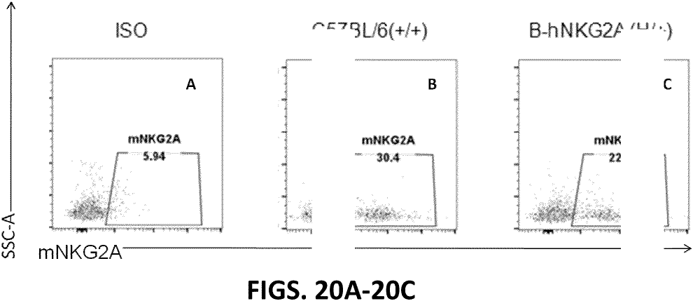

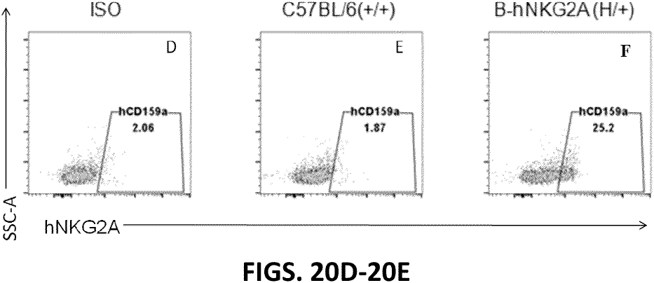

The expression of humanized NKG2A protein in the positive mice was confirmed, e.g., using the Fluorescence-Activated Cell Sorting (FACS) method, and the detection was performed as follows. One wild-type C57BL/6 mouse and one NKG2A gene humanized heterozygous mouse were selected. The mouse spleen cells were stained with Brilliant Violet 510™ anti-mouse CD45 (an anti-mouse CD45 antibody), PE/Cy™ 7 Mouse anti-mouse NK1.1 (an anti-mouse NK cell surface antigen antibody), and either one of: (1) mNKG2A-PE (using PE Mouse IgG2b, κ Isotype Ctrl Antibody as an isotype control (ISO)) (FIGS. 20A-20C), or (2) monalizumab in combination of Alexa Fluor® 647-conjugated AffiniPure F(ab′)2 Fragment Goat Anti-Human IgG, Fcy fragment Specific (using Alexa Fluor® 647 Mouse IgG1, κ Isotype Ctrl (FC) as an isotype control (ISO)) (FIGS. 20D-20E), followed by flow cytometry detection. The results showed that no humanized NKG2A protein expression, but only mouse NKG2A protein expression, was detected in wild-type C57BL/6 mice (FIG. 20B and FIG. 20E). By contrast, both mouse NKG2A protein and humanized NKG2A protein expression were detected in NKG2A gene humanized heterozygous mice (FIG. 20C and FIG. 20F). The above experiments showed that NKG2A gene humanized mice expressing humanized NKG2A protein in vivo can be established using the methods described herein, and the mice can be stably passed without random insertions.

### Example 3: Generation of Double- or Multi-Gene Humanized Mice

The CD94 gene humanized mice generated in Example 1 and the NKG2A gene humanized mice generated in Example 2 can also be used to generate double- or multi-gene humanized mouse model. For example, in Example 2, the embryonic stem (ES) cells used for blastocyst microinjection can be selected from the CD94 gene humanized positive clone cells in Example 1, thereby generating NKG2A and CD94 double-gene humanized mice. In addition, it is also possible to breed the homozygous or heterozygous NKG2A and/or CD94 gene humanized mice obtained by the methods described herein with other genetically modified homozygous or heterozygous mice, and the offspring can be screened. According to Mendel’s law, it is possible to generate double-gene or multi-gene modified heterozygous mice comprising humanized NKG2A and/or CD94 genes and other genetic modifications. Then the heterozygous mice can be bred with each other to obtain homozygous double-gene or multi-gene humanized mice.

For example, NKG2A/CD94 double-gene humanized mice (B-hCD94/hNKG2A) can be generated using the above method. Because mouse NKG2A and CD94 genes are both located on chromosome 6, after obtaining the CD94 gene humanized positive ES cells, the method as described in Example 2 was performed to conduct a second round of targeting. Afterwards, the positive offspring can be screened, thereby generating NKG2A/CD94 double-gene humanized mice.

RT-PCR can be used to detect the expression of humanized NKG2A mRNA and humanized CD94 mRNA in NKG2A/CD94 double-gene humanized mice. Specifically, three wild-type C57BL/6 mice (7 weeks old) and three NKG2A/CD94 double-gene humanized homozygous mice (7 weeks old) were selected and mouse spleen was isolated after euthanasia. Total RNA from the spleen was extracted and then reverse transcribed into cDNA using a reverse transcription kit, followed by PCR amplification. The primer sequence are shown in the table below.

TABLE 11

RT-PCR detection primer sequences and target fragment sizes

| | | |
|---|---|---|
| Primer | Primer sequence (5′-3′) | Target fragment size |
| hCD94-RT-PCR-F2 | CCAGCATTTACTCCAGGACCC (SEQ ID NO: 24) | Mut: 288 bp |
| hCD94-RT-PCR-R2 | GGAGAGTGCAGAGCCATTCT (SEQ ID NO: 25) | |
| mCD94-RT-PCR-F1 | AGTTTCTAGGATCACTCGGTGG (SEQ ID NO: 26) | WT: 452 bp |
| mCD94-RT-PCR-R1 | TGCAGTGCTCTGGCCTGATA (SEQ ID NO: 27) | |
| hNKG2A-RT-PCR-F1 | CTCGCAGCTCCATTTCAGTC (SEQ ID NO: 59) | Mut: 245 bp |
| hNKG2A-RT-PCR-R1 | CAGGGAAGAATTGTTGTGCCT (SEQ ID NO: 60) | |
| mNKG2A-RT-PCR-F1 | ATGCAGAACTGAAGGTGGCA (SEQ ID NO: 61) | WT: 661 bp |
| mNKG2A-RT-PCR-R1 | TCTGCTGTGAGACCAGAAGC (SEQ ID NO: 62) | |
| GAPDH-F | TCACCATCTTCCAGGAGCGAGA (SEQ ID NO: 63) | WT: 479 bp |
| GAPDH-R | GAAGGCCATGCCAGTGAGCTT (SEQ ID NO: 64) | |

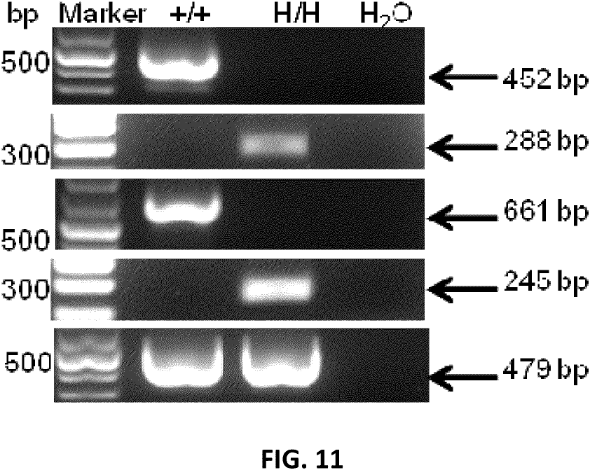

As shown in FIG. 11, the test results showed that in wild-type C57BL/6 mouse cells, expression of mouse NKG2A and CD94 mRNA was detected, but expression of humanized NKG2A and CD94 mRNA was not detected. By contrast, in humanized NKG2A/CD94 double-gene humanized homozygous mouse cells, expression of humanized NKG2A and CD94 mRNA was detected, but expression of mouse NKG2A and CD94 mRNA was not detected.

Further, flow cytometry was used to detect the in vivo expression of NKG2A and CD94 proteins in NKG2A/CD94 double-gene humanized mice. Specifically, one wild-type C57BL/6 mouse (9 weeks old) and one NKG2A/CD94 double-gene humanized homozygous mouse (9 weeks old) were selected and mouse spleen was isolated after euthanasia.

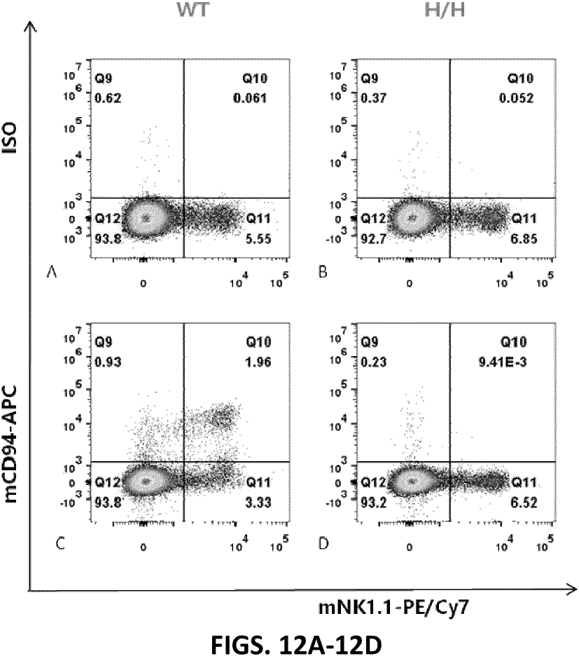

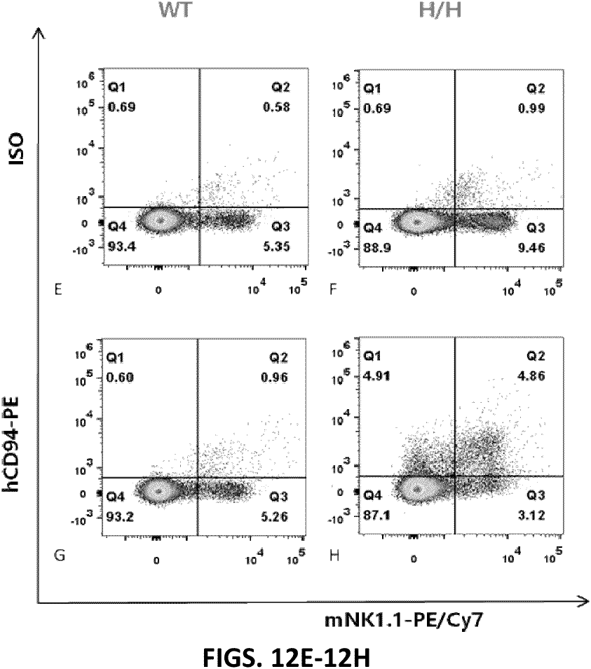

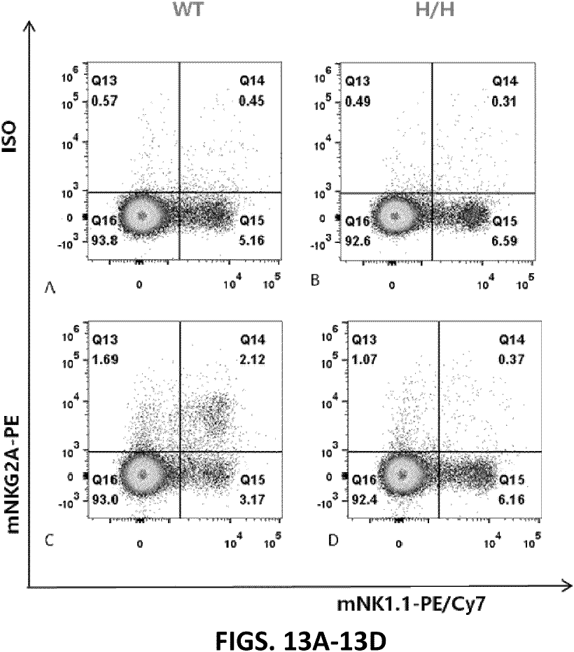

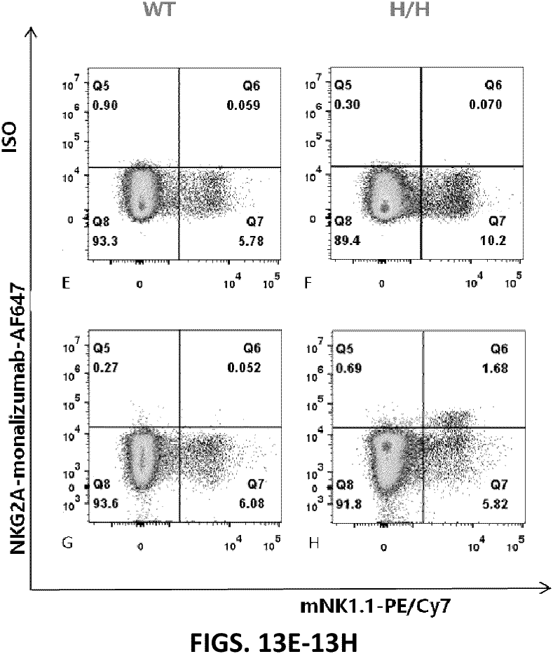

The spleen cells were stained with anti-mouse CD45 antibody Brilliant Violet 510™ anti-mouse CD45 and mNK1.1-PE/Cy7, together with (1) mCD94-APC; (2) hCD94-PE; (3) mNKG2A-PE; and/or (4) monalizumab in combination with Alexa Fluor® 647-conjugated AffiniPure F(ab′)2 Fragment Goat Anti-Human IgG, Fcy fragment Specific. APC Mouse IgG2a, κ Isotype Ctrl Antibody, PE Mouse IgG2b, κ Isotype Ctrl Antibody, or APC Rat IgG2a, κ Isotype Ctrl Antibody were used as isotype controls. As shown in FIGS. 12A-12H, 13A-13H, and 14A-14H, the results showed that in wild-type C57BL/6 mice, mouse NKG2A protein and CD94 protein were detected, but humanized NKG2A protein and CD94 protein were not detected. By contrast, in NKG2A/CD94 double-gene humanized homozygous mice, humanized NIG2A protein and CD94 protein were detected, but mouse NKG2A and CD94 were not detected.

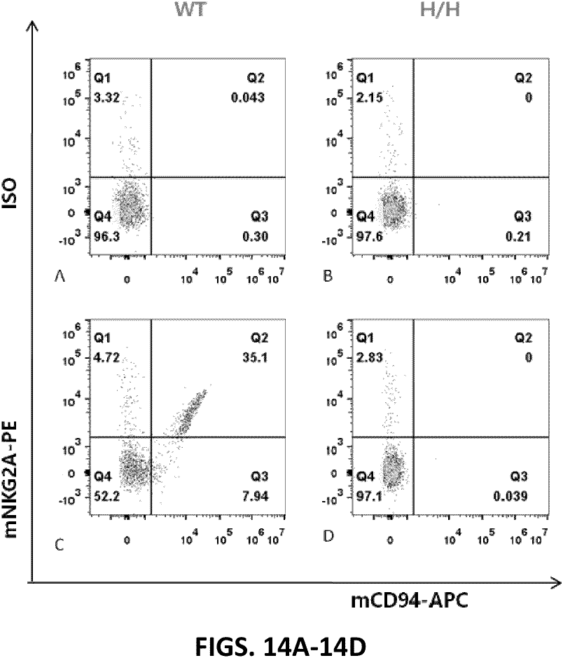

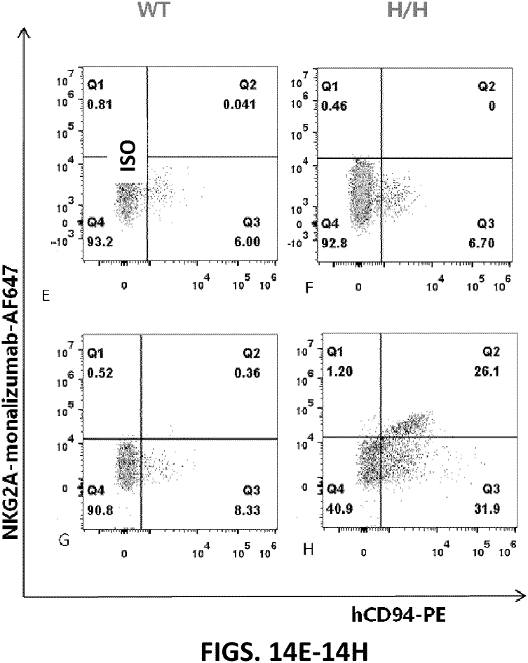

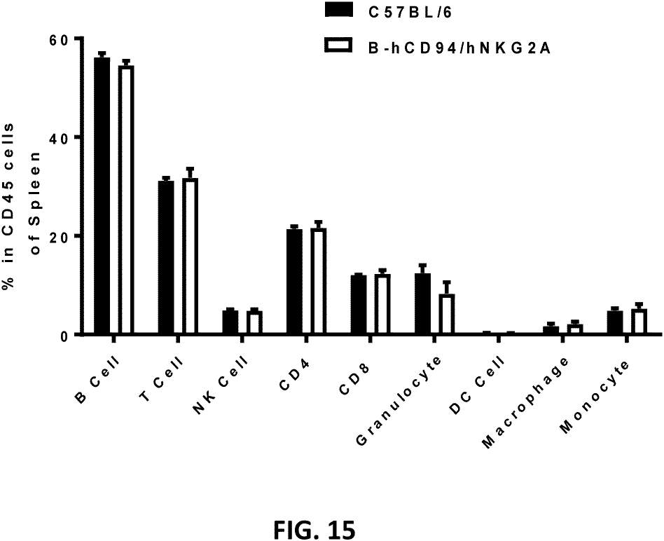

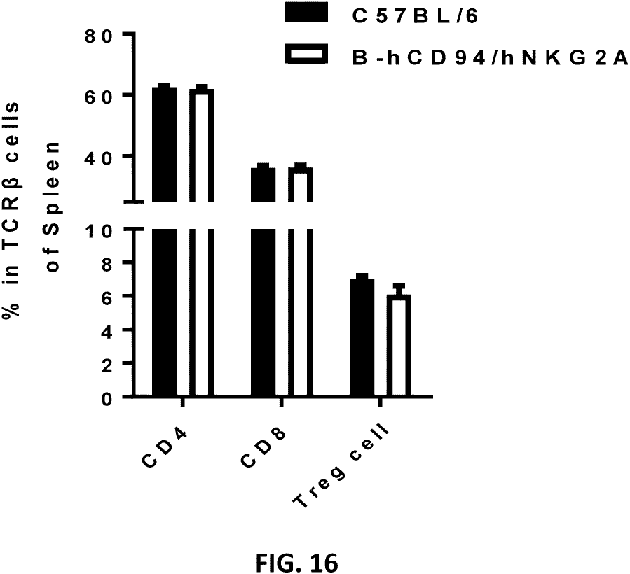

Further, immuno-phenotyping of leukocytes and T cells in the spleen of wild-type C57BL/6 mice and NKG2A/CD94 double-gene humanized homozygous mice was performed by flow cytometry. The immuno-phenotyping detection results of leukocytes and T cells in the spleen are shown in FIG. 15 and FIG. 16, respectively. The results showed that leukocyte subtypes, e.g., T cells, B cells, NK cells, CD4+ T cells, CD8+ T cells, granulocytes, DC cells, macrophages, and monocytes in NKG2A/CD94 double-gene humanized homozygous mice had comparable levels as compared to those in wild-type C57BL/6 mice (FIG. 14). The results also showed that the percentages of T cell subtypes, e.g., CD4+ T cells, CD8+ T cells, and Treg cells in NKG2A/CD94 double-gene humanized homozygous mice, were comparable with those detected in wild-type C57BL/6 mice (FIG. 15). The above results indicate that modification (e.g., humanization) of the mouse NKG2A/CD94 gene did not affect the differentiation, development, and distribution of leukocytes in the lymphatic tissues of mice.

The above results showed that NKG2A/CD94 double-gene humanized mice expressing humanized NKG2A protein and humanized CD94 protein can be successfully constructed.

In addition, the CD94 and/or NKG2A gene humanized mice as described herein can also be used to generate triple- or multi-gene humanized mice. For example, NKG2A/CD94/PD-1 triple-gene humanized mice can be generated. Because mouse PD-1 gene is located on chromosome 1, NKG2A/CD94 double-gene humanized mice can be selected for breeding with PD-1 gene humanized mice, and the positive offspring can be screened, thereby generating NKG2A/CD94/PD-1 triple-gene humanized mice.

### Example 4.: In Vivo Drug Efficacy Verification in Animal Models

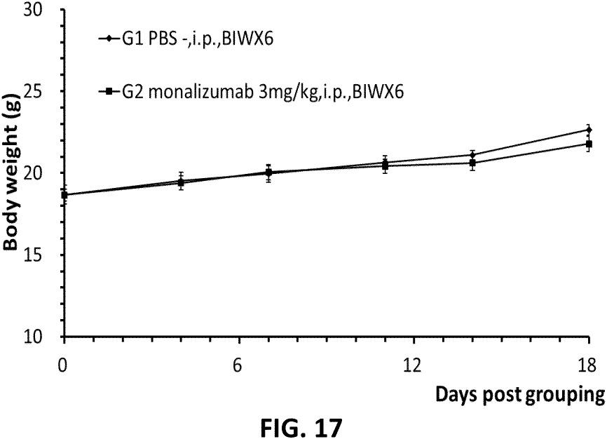

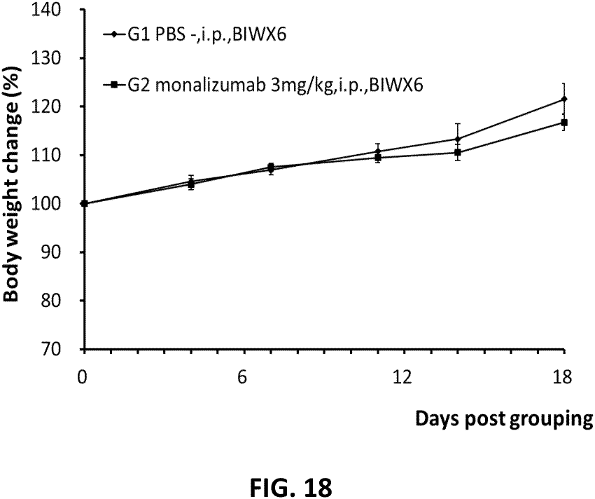

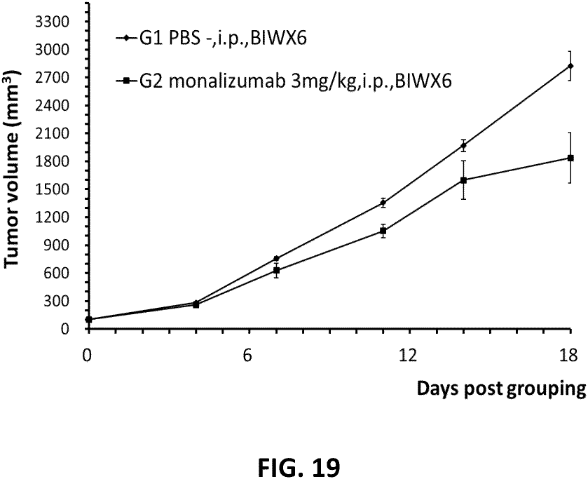

The NKG2A and/or CD94 gene humanized mice as described herein can be used to establish mouse tumor models for testing the efficacy of drugs targeting human NKG2A and/or CD94. Specifically, the NKG2A/CD94 double-gene humanized homozygous mice (8 weeks old) as prepared in Example 3 were subcutaneously injected with 5 × 10⁵ mouse colon cancer cell B-CAG-hHLA-E MC38 (i.e., MC38 cells expressing human HLA). When the tumor volume grew to about 100 mm³, the mice were randomly placed into a control group (G1) and a treatment group (G2) based on tumor size (5 mice per group). The treatment group mice were administered with the anti-human NKG2A antibody monalizumab via intraperitoneal injection (i.p.), whereas the control group mice were injected with an equal volume of phosphate-buffered saline (PBS). The mice were administered on the grouping day, and the frequency of administration was twice a week (6 times of administrations in total). The tumor volume was measured twice a week and the body weight of the mice was weighed as well. Euthanasia was performed when the tumor volume of the mouse reached 3000 mm³. The table below shows grouping and administration details. The mouse body weight, body weight change, and tumor volume measurement results during the experimental period are shown in FIG. 17, FIG. 18, and FIG. 19, respectively.

TABLE 12

| Group | | Animal number | Treatment | Dose level | Administra tion route | Administration frequency and total times |
|---|---|---|---|---|---|---|
| Control | G1 | 5 | PBS | - | i.p. | BIW × 6 |
| Treatment | G2 | 5 | monalizumab | 3 mg/kg | i.p. | BIW × 6 |

The table below lists the major data and analysis results of each experiment, including: the tumor volume at the time of grouping (Day 0), 11 days post grouping (Day 11), and 18 days post grouping (Day 18); number of survived mice and tumor-free mice at the end of the experiment; tumor growth inhibition value (TGI_{TV}%); and the statistical difference (P value) of body weight and tumor volume between the treatment group and the control group.

TABLE 13

| Group | Tumor volume (mm³) | Survived mice on Day 18 | Tumor-free mice on Day 18 | TGI_{TV}% | P value | | | |
|---|---|---|---|---|---|---|---|---|
| | Day 0 | | | | Day 18 | | Body weight | Tumor volume |
| G1 | 102±3 | 1355±49 | 2825±156 | 5/5 | 0/5 | N/A | N/A | N/A |
| G2 | 102±4 | 1053±71 | 1837±273 | 5/5 | 0/5 | 36.3% | 0.173 | 0.014 |

Overall, the animals in each group were healthy, and the body weights of all the treatment group mice (G2) and control group mice (G1) increased, and were not significantly different (P > 0.05) from each other during the experimental period (FIG. 17 and FIG. 18). The results indicate that the treatment group mice tolerated the anti-human NKG2A antibody monalizumab well. According to the tumor volume measurement results shown in FIG. 19 and the table above, the tumor volume of the treatment group mice was smaller than that of the control group mice in each period of the experiment. Specifically on Day 18, the tumor volume of the G2 group mice was 1837 ± 273 mm³, which was significantly reduced as compared to the tumor volume of 2825 ± 156 mm³ of the control group mice (P < 0.05), indicating that 3 mg/kg of monalizumab has a good tumor growth inhibition effect in NKG2A/CD94 double-gene humanized animals.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetically-modified, non-human animal whose genome comprises at least one chromosome comprising a sequence encoding a human or chimeric CD94.

2. The animal of claim 1, wherein the sequence encoding the human or chimeric CD94 is operably linked to an endogenous regulatory element at the endogenous CD94 gene locus in the at least one chromosome.

3. The animal of claim 1 or 2, wherein the sequence encoding a human or chimeric CD94 comprises a sequence encoding an amino acid sequence that is at least 70%, 75%, 80%, 85%, 90%, 95%, 99%, or 100% identical to human CD94 (NP_001337991.1 (SEQ ID NO: 2)).

4. The animal of claim 1 or 2, wherein the sequence encoding a human or chimeric CD94 comprises a sequence encoding an amino acid sequence that is at least 70%, 75%, 80%, 85%, 90%, 95%, 99%, or 100% identical to SEQ ID NO: 8.

5. The animal of claim 1 or 2, wherein the sequence encoding a human or chimeric CD94 comprises a sequence that is at least 70%, 75%, 80%, 85%, 90%, 95%, 99%, or 100% identical to amino acids of 37-179 of SEQ ID NO: 2.

6. The animal of any one of claims 1-5, wherein the animal is a mammal, e.g., a monkey, a rodent, or a mouse.

7. The animal of any one of claims 1-6, wherein the animal is a mouse.

8. The animal of any one of claims 1-7, wherein the animal does not express endogenous CD94 or expresses a decreased level of endogenous CD94.

9. The animal of any one of claims 1-8, wherein the animal has one or more cells expressing human or chimeric CD94.

10. The animal of any one of claims 1-9, wherein the animal has one or more cells expressing human or chimeric CD94, and a human NKG2A can interact with the expressed human or chimeric CD94, forming a heterodimer that can recognize MHC class I molecules.

11. The animal of any one of claims 1-9, wherein the animal has one or more cells expressing human or chimeric CD94, and an endogenous NKG2A can interact with the expressed human or chimeric CD94, forming a heterodimer that can recognized MHC class I molecules.

12. A genetically-modified, non-human animal, wherein the genome of the animal comprises a replacement of a sequence encoding a region of endogenous CD94 with a sequence encoding a corresponding region of human CD94 at an endogenous CD94 gene locus.

13. The animal of claim 12, wherein the sequence encoding the corresponding region of human CD94 is operably linked to an endogenous regulatory element at the endogenous CD94 locus, and one or more cells of the animal expresses a chimeric CD94.

14. The animal of claim 12 or 13, wherein the animal does not express endogenous CD94.

15. The animal of any one of claims 12-14, wherein the replaced sequence encodes all or a portion of the extracellular region of endogenous CD94.

16. The animal of any one of claims 12-15, wherein the animal has one or more cells expressing a chimeric CD94 having a cytoplasmic region, a transmembrane region, and an extracellular region, wherein the extracellular region comprises a sequence that is at least 50%, 60%, 70%, 80%, 90%, 95%, or 99% identical to the extracellular region of human CD94.

17. The animal of claim 16, wherein the extracellular region of the chimeric CD94 has a sequence that has at least 10, 20, 30, 40, 50, 60, 70, 80, 90, 100, 110, 120, 130, or 140 contiguous amino acids that are identical to a contiguous sequence present in the extracellular region of human CD94.

18. The animal of any one of claims 12-17, wherein the sequence encoding a region of endogenous CD94 comprises exon 1, exon 2, exon 3, exon 4, exon 5, and/or exon 6, or a part thereof, of the endogenous CD94 gene.

19. The animal of claim 18, wherein the animal is a mouse, and the sequence encoding a region of endogenous CD94 starts within exon 3 and ends within exon 6 of the endogenous mouse CD94 gene.

20. The animal of any one of claims 12-19, wherein the animal is heterozygous with respect to the replacement at the endogenous CD94 gene locus.

21. The animal of any one of claims 12-19, wherein the animal is homozygous with respect to the replacement at the endogenous CD94 gene locus.

22. A method for making a genetically-modified, non-human animal, comprising:
replacing in at least one cell of the animal, at an endogenous CD94 gene locus, a sequence encoding a region of an endogenous CD94 with a sequence encoding a corresponding region of human CD94.

23. The method of claim 22, wherein the sequence encoding the corresponding region of human CD94 comprises exon 2, exon 3, exon 4, exon 5, exon 6, and/or exon 7, or a part thereof, of a human CD94 gene.

24. The method of claim 22 or 23, wherein the sequence encoding the corresponding region of human CD94 starts within exon 4 and ends within exon 7 of a human CD94 gene.

25. The method of any one of claims 22-24, wherein the sequence encoding the corresponding region of human CD94 encodes amino acids 37-179 of SEQ ID NO: 2.

26. The method of any one of claims 22-25, wherein the region of an endogenous CD94 is located within the extracellular region.

27. The method of any one of claims 22-26, wherein the sequence encoding a region of endogenous CD94 comprises exon 1, exon 2, exon 3, exon 4, exon 5, and/or exon 6, or a part thereof, of the endogenous CD94 gene.

28. The method of claim 27, wherein the animal is a mouse, and the sequence encoding a region of an endogenous CD94 starts within exon 3 and ends within exon 6 of the endogenous mouse CD94 gene.

29. A non-human animal comprising at least one cell comprising a nucleotide sequence encoding a chimeric CD94 polypeptide, wherein the chimeric CD94 polypeptide comprises at least 50 contiguous amino acid residues that are identical to the corresponding contiguous amino acid sequence of a human CD94, wherein the animal expresses the chimeric CD94.

30. The animal of claim 29, wherein the chimeric CD94 polypeptide has at least 50, at least 60, at least 70, at least 80, at least 90, at least 100, at least 110, at least 120, at least 130, or at least 140 contiguous amino acid residues that are identical to the corresponding contiguous amino acid sequence of a human CD94 extracellular region.

31. The animal of claim 29 or 30, wherein the chimeric CD94 polypeptide comprises a sequence that is at least 90%, 95%, or 99% identical to amino acids 37-179 of SEQ ID NO: 2.

32. The animal of any one of claims 29-31, wherein the nucleotide sequence is operably linked to an endogenous CD94 regulatory element of the animal.

33. The animal of any one of claims 29-32, wherein the chimeric CD94 polypeptide comprises an endogenous CD94 cytoplasmic region and/or an endogenous CD94 transmembrane region.

34. The animal of any one of claims 29-33, wherein the nucleotide sequence is integrated to an endogenous CD94 gene locus of the animal.

35. The animal of any one of claims 29-34, wherein the chimeric CD94 has at least one mouse CD94 activity and/or at least one human CD94 activity.

36. A method of making a genetically-modified non-human animal cell that expresses a chimeric CD94, the method comprising:
replacing at an endogenous CD94 gene locus, a nucleotide sequence encoding a region of endogenous CD94 with a nucleotide sequence encoding a corresponding region of human CD94, thereby generating a genetically-modified non-human animal cell that includes a nucleotide sequence that encodes the chimeric CD94, wherein the non-human animal cell expresses the chimeric CD94.

37. The method of claim 36, wherein the animal is a mammal, e.g., a monkey, a rodent, or a mouse.

38. The method of claim 36 or 37, wherein the chimeric CD94 comprises:
a cytoplasmic region and/or a transmembrane region of endogenous CD94; and
an extracellular region of human CD94.

39. The method of any one of claims 36-38, wherein the nucleotide sequence encoding the chimeric CD94 is operably linked to an endogenous CD94 regulatory region, e.g., promoter.

40. The animal of any one of claims 1-21 and 29-35, wherein the animal further comprises a sequence encoding an additional human or chimeric protein.

41. The animal of claim 40, wherein the additional human or chimeric protein is Killer Cell Lectin Like Receptor C1 (NKG2A), programmed cell death protein 1 (PD-1), B7 Homolog 3 (B7-H3), V-set domain-containing T-cell activation inhibitor 1 (B7-H4), Interleukin-2 (IL-2), Interleukin-23 subunit alpha (IL23A), C-C Motif Chemokine Receptor 2 (CCR2), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α (SIRPα) or TNF Receptor Superfamily Member 4 (OX40).

42. The animal of claim 40, wherein the additional human or chimeric protein is NKG2A, and the animal expresses the human or chimeric NKG2A.

43. The animal of claim 42, wherein the animal further comprises a sequence encoding human or chimeric PD-1, and the animal expresses the human or chimeric PD-1.

44. The method of any one of claims 22-28 and 36-39, wherein the animal or mouse further comprises a sequence encoding an additional human or chimeric protein.

45. The method of claim 44, wherein the additional human or chimeric protein is NKG2A, PD-1, B7-H3, B7-H4, IL-2, IL23A, CCR2, CTLA-4, LAG-3, BTLA, PD-L1, CD27, CD28, CD47, CD137, CD154, TIGIT, TIM-3, GITR, SIRPα or OX40.

46. The method of claim 44, wherein the additional human or chimeric protein is NKG2A, and the animal expresses the human or chimeric NKG2A.

47. The method of claim 46, wherein the animal further comprises a sequence encoding human or chimeric PD-1, and the animal expresses the human or chimeric PD-1.

48. A genetically-modified, non-human animal whose genome comprises at least one chromosome comprising a sequence encoding a human or chimeric Killer cell lectin-like receptor subfamily C, member 1 (NKG2A).

49. The animal of claim 48, wherein the sequence encoding the human or chimeric NKG2A is operably linked to an endogenous regulatory element at the endogenous NKG2A gene locus in the at least one chromosome.

50. The animal of claim 48 or 49, wherein the sequence encoding a human or chimeric NKG2A comprises a sequence encoding an amino acid sequence that is at least 70%, 75%, 80%, 85%, 90%, 95%, 99%, or 100% identical to human NKG2A (NP_998823.1 (SEQ ID NO: 29)).

51. The animal of claim 48 or 49, wherein the sequence encoding a human or chimeric NKG2A comprises a sequence encoding an amino acid sequence that is at least 70%, 75%, 80%, 85%, 90%, 95%, 99%, or 100% identical to SEQ ID NO: 35.

52. The animal of claim 48 or 49, wherein the sequence encoding a human or chimeric NKG2A comprises a sequence that is at least 70%, 75%, 80%, 85%, 90%, 95%, 99%, or 100% identical to amino acids of 94-233 of SEQ ID NO: 29.

53. The animal of any one of claims 48-52, wherein the animal is a mammal, e.g., a monkey, a rodent, or a mouse.

54. The animal of any one of claims 48-53, wherein the animal is a mouse.

55. The animal of any one of claims 48-54, wherein the animal does not express endogenous NKG2A or expresses a decreased level of endogenous NKG2A.

56. The animal of any one of claims 48-55, wherein the animal has one or more cells expressing human or chimeric NKG2A.

57. The animal of any one of claims 48-56, wherein the animal has one or more cells expressing human or chimeric NKG2A, and a human CD94 can interact with the expressed human or chimeric NKG2A, forming a heterodimer that can recognize MHC class I molecules.

58. The animal of any one of claims 48-56, wherein the animal has one or more cells expressing human or chimeric NKG2A, and an endogenous CD94 can interact with the expressed human or chimeric NKG2A, forming a heterodimer that can recognized MHC class I molecules.

59. A genetically-modified, non-human animal, wherein the genome of the animal comprises a replacement of a sequence encoding a region of endogenous NKG2A with a sequence encoding a corresponding region of human NKG2A at an endogenous NKG2A gene locus.

60. The animal of claim 59, wherein the sequence encoding the corresponding region of human NKG2A is operably linked to an endogenous regulatory element at the endogenous NKG2A locus, and one or more cells of the animal expresses a chimeric NKG2A.

61. The animal of claim 59 or 60, wherein the animal does not express endogenous NKG2A.

62. The animal of any one of claims 59-61, wherein the replaced sequence encodes all or a portion of the extracellular region of endogenous NKG2A.

63. The animal of any one of claims 59-62, wherein the animal has one or more cells expressing a chimeric NKG2A having a cytoplasmic region, a transmembrane region, and an extracellular region, wherein the extracellular region comprises a sequence that is at least 50%, 60%, 70%, 80%, 90%, 95%, or 99% identical to the extracellular region of human NKG2A.

64. The animal of claim 63, wherein the extracellular region of the chimeric NKG2A has a sequence that has at least 10, 20, 30, 40, 50, 60, 70, 80, 90, 100, 110, 120, 130, or 140 contiguous amino acids that are identical to a contiguous sequence present in the extracellular region of human NKG2A.

65. The animal of any one of claims 59-64, wherein the sequence encoding a region of endogenous NKG2A comprises exon 1, exon 2, exon 3, exon 4, exon 5, and/or exon 6, or a part thereof, of the endogenous NKG2A gene.

66. The animal of claim 65, wherein the animal is a mouse, and the sequence encoding a region of endogenous NKG2A starts within exon 2 and ends within exon 6 of the endogenous mouse NKG2A gene.

67. The animal of any one of claims 59-66, wherein the animal is heterozygous with respect to the replacement at the endogenous NKG2A gene locus.

68. The animal of any one of claims 59-66, wherein the animal is homozygous with respect to the replacement at the endogenous NKG2A gene locus.

69. A method for making a genetically-modified, non-human animal, comprising:
replacing in at least one cell of the animal, at an endogenous NKG2A gene locus, a sequence encoding a region of an endogenous NKG2A with a sequence encoding a corresponding region of human NKG2A.

70. The method of claim 69, wherein the sequence encoding the corresponding region of human NKG2A comprises exon 3, exon 4, exon 5, exon 6, exon 7, and/or exon 8, or a part thereof, of a human NKG2A gene.

71. The method of claim 69 or 70, wherein the sequence encoding the corresponding region of human NKG2A starts within exon 4 and ends within exon 8 of a human NKG2A gene.

72. The method of any one of claims 69-71, wherein the sequence encoding the corresponding region of human NKG2A encodes amino acids 94-233 of SEQ ID NO: 29.

73. The method of any one of claims 69-72, wherein the region of an endogenous NKG2A is located within the extracellular region.

74. The method of any one of claims 69-73, wherein the sequence encoding a region of endogenous NKG2A comprises exon 1, exon 2, exon 3, exon 4, exon 5, and/or exon 6, or a part thereof, of the endogenous NKG2A gene.

75. The method of claim 74, wherein the animal is a mouse, and the sequence encoding a region of an endogenous NKG2A starts within exon 2 and ends within exon 6 of the endogenous mouse NKG2A gene.

76. A non-human animal comprising at least one cell comprising a nucleotide sequence encoding a chimeric NKG2A polypeptide, wherein the chimeric NKG2A polypeptide comprises at least 50 contiguous amino acid residues that are identical to the corresponding contiguous amino acid sequence of a human NKG2A, wherein the animal expresses the chimeric NKG2A.

77. The animal of claim 76, wherein the chimeric NKG2A polypeptide has at least 50, at least 60, at least 70, at least 80, at least 90, at least 100, at least 110, at least 120, at least 130, or at least 140 contiguous amino acid residues that are identical to the corresponding contiguous amino acid sequence of a human NKG2A extracellular region.

78. The animal of claim 76 or 77, wherein the chimeric NKG2A polypeptide comprises a sequence that is at least 90%, 95%, or 99% identical to amino acids 94-233 of SEQ ID NO: 29.

79. The animal of any one of claims 76-78, wherein the nucleotide sequence is operably linked to an endogenous NKG2A regulatory element of the animal.

80. The animal of any one of claims 76-79, wherein the chimeric NKG2A polypeptide comprises an endogenous NKG2A cytoplasmic region and/or an endogenous NKG2A transmembrane region.

81. The animal of any one of claims 76-80, wherein the nucleotide sequence is integrated to an endogenous NKG2A gene locus of the animal.

82. The animal of any one of claims 76-81, wherein the chimeric NKG2A has at least one mouse NKG2A activity and/or at least one human NKG2A activity.

83. A method of making a genetically-modified non-human animal cell that expresses a chimeric NKG2A, the method comprising:
replacing at an endogenous NKG2A gene locus, a nucleotide sequence encoding a region of endogenous NKG2A with a nucleotide sequence encoding a corresponding region of human NKG2A, thereby generating a genetically-modified non-human animal cell that includes a nucleotide sequence that encodes the chimeric NKG2A, wherein the non-human animal cell expresses the chimeric NKG2A.

84. The method of claim 83, wherein the animal is a mammal, e.g., a monkey, a rodent, or a mouse.

85. The method of claim 83 or 84, wherein the chimeric NKG2A comprises:
a cytoplasmic region and/or a transmembrane region of endogenous NKG2A; and
an extracellular region of human NKG2A.

86. The method of any one of claims 83-85, wherein the nucleotide sequence encoding the chimeric NKG2A is operably linked to an endogenous NKG2A regulatory region, e.g., promoter.

87. The animal of any one of claims 48-68 and 76-82, wherein the animal further comprises a sequence encoding an additional human or chimeric protein.

88. The animal of claim 87, wherein the additional human or chimeric protein is Cluster of Differentiation 94 (CD94), programmed cell death protein 1 (PD-1), B7 Homolog 3 (B7-H3), V-set domain-containing T-cell activation inhibitor 1 (B7-H4), Interleukin-2 (IL-2), Interleukin-23 subunit alpha (IL23A), C-C Motif Chemokine Receptor 2 (CCR2), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α (SIRPα) or TNF Receptor Superfamily Member 4 (OX40).

89. The animal of claim 87, wherein the additional human or chimeric protein is CD94, and the animal expresses the human or chimeric CD94.

90. The animal of claim 89, wherein the animal further comprises a sequence encoding human or chimeric PD-1, and the animal expresses the human or chimeric PD-1.

91. The method of any one of claims 69-75 and 83-86, wherein the animal or mouse further comprises a sequence encoding an additional human or chimeric protein.

92. The method of claim 91, wherein the additional human or chimeric protein is CD94, PD-1, B7-H3, B7-H4, IL-2, IL23A, CCR2, CTLA-4, LAG-3, BTLA, PD-L1, CD27, CD28, CD47, CD137, CD154, TIGIT, TIM-3, GITR, SIRPα or OX40.

93. The method of claim 91, wherein the additional human or chimeric protein is CD94, and the animal expresses the human or chimeric CD94.

94. The method of claim 93, wherein the animal further comprises a sequence encoding human or chimeric PD-1, and the animal expresses the human or chimeric PD-1.

95. A method of determining effectiveness of a therapeutic agent recognizing a NKG2A/CD94 receptor for treating cancer, comprising:
administering the therapeutic agent to the animal of any one of claims 1-21, 29-35, 40-43, 48-68, 76-82, and 87-90, wherein the animal has a cancer; and
determining the inhibitory effects of the therapeutic agent to the cancer.

96. The method of claim 95, wherein the therapeutic agent is an anti-CD94 antibody or anti-NKG2A antibody.

97. The method of claim 95 or 96, wherein the cancer comprises one or more cells that express MHC class I molecules (e.g., human HLA-E or mouse Qa-1).

98. The method of any one of claims 95-97, wherein the cancer comprises one or more cancer cells that are injected into the animal.

99. The method of any one of claims 95-98, wherein determining the inhibitory effects of the therapeutic agent to the cancer involves measuring the tumor volume in the animal.

100. The method of any one of claims 95-99, wherein the cancer is gynecologic cancer, ovarian cancer, uterine cancer, vaginal cancer, cervical cancer, vulvar cancer, head and neck cancer, non-small cell lung cancer (NSCLC), hematological cancer, solid tumor, breast cancer, chronic lymphocytic leukemia, squamous cell carcinoma of the oral cavity, colorectal cancer, liver cancer, glioblastoma, Hodgkin lymphoma, esophagus cancer, gastric cancer, pancreas cancer, renal cancer, lung cancer, or melanoma.

101. A method of determining effectiveness of an therapeutic agent recognizing a NKG2A/CD94 receptor and an additional therapeutic agent for the treating cancer, comprising
administering the therapeutic agent and the additional therapeutic agent to the animal of any one of claims 1-21, 29-35, 40-43, 48-68, 76-82, and 87-90, wherein the animal has a cancer; and
determining the inhibitory effects on the cancer.

102. The method of claim 101, wherein the animal further comprises a sequence encoding a human or chimeric programmed cell death protein 1 (PD-1).

103. The method of claim 101 or 102, wherein the animal further comprises a sequence encoding a human or chimeric programmed death-ligand 1 (PD-L1).

104. The method of any one of claims 101-103, wherein the additional therapeutic agent is an anti-PD-1 antibody, an anti-PD-L1 antibody, or an anti-EGFR (epidermal growth factor receptor) antibody..

105. The method of claim any one of claims 101-104, wherein the cancer comprises one or more cancer cells that express PD-L1, or PD-L2.

106. The method of any one of claims 101-105, wherein the cancer is caused by injection of one or more cancer cells into the animal.

107. The method of any one of claims 101-106, wherein determining the inhibitory effects of the treatment involves measuring the tumor volume in the animal.

108. The method of any one of claims 101-107, wherein the animal has gynecologic cancer, ovarian cancer, uterine cancer, vaginal cancer, cervical cancer, vulvar cancer, head and neck cancer, non-small cell lung cancer (NSCLC), hematological cancer, solid tumor, breast cancer, chronic lymphocytic leukemia, squamous cell carcinoma of the oral cavity, colorectal cancer, liver cancer, glioblastoma, Hodgkin lymphoma, esophagus cancer, gastric cancer, pancreas cancer, renal cancer, lung cancer, or melanoma.

109. A method of determining effectiveness of a therapeutic agent recognizing a NKG2A/CD94 receptor for treating an immune disorder, comprising:
administering the therapeutic agent to the animal of any one of claims 1-21, 29-35, 40-43, 48-68, 76-82, and 87-90, wherein the animal has the immune disorder; and
determining the inhibitory effects of the therapeutic agent to the immune disorder.

110. The method of claim 109, wherein the therapeutic agent is an anti-CD94 antibody or anti-NKG2A antibody.

111. The method of claim 109 or 110, wherein the cancer comprises one or more cells that express MHC class I molecules (e.g., human HLA-E or mouse Qa-1).

112. The method of any one of claims 109-111, wherein the immune disorder is rheumatoid arthritis.

113. A protein comprising an amino acid sequence, wherein the amino acid sequence is one of the following:
(a) an amino acid sequence set forth in SEQ ID NO: 1, 2, 8, 28, 29, or 35;
(b) an amino acid sequence that is at least 90% identical to SEQ ID NO: 1, 2, 8, 28, 29, or 35;
(c) an amino acid sequence that is at least 91%, 92%, 93%, 94%, 95%, 96%, 97%, 98%, or 99% identical to SEQ ID NO: 1, 2, 8, 28, 29, or 35;
(d) an amino acid sequence that is different from the amino acid sequence set forth in SEQ ID NO: 1, 2, 8, 28, 29, or 35 by no more than 10, 9, 8, 7, 6, 5, 4, 3, 2 or 1 amino acid; and
(e) an amino acid sequence that comprises a substitution, a deletion and /or insertion of one, two, three, four, five or more amino acids to the amino acid sequence set forth in SEQ ID NO: 1, 2, 8, 28, 29, or 35.

114. A nucleic acid comprising a nucleotide sequence, wherein the nucleotide sequence is one of the following:
(a) a sequence that encodes the protein of claim 113;
(b) SEQ ID NO: 3, 4, 5, 6, 7, 9, 10, 30, 31, 32, 33, 34, 36, or 37;
(c) a sequence that is at least 90% identical to SEQ ID NO: 3, 4, 5, 6, 7, 9, 10, 30, 31, 32, 33, 34, 36, or 37; and
(d) a sequence that is at least 91%, 92%, 93%, 94%, 95%, 96%, 97%, 98%, or 99% identical to SEQ ID NO: 3, 4, 5, 6, 7, 9, 10, 30, 31, 32, 33, 34, 36, or 37.

115. A cell comprising the protein of claim 113 and/or the nucleic acid of claim 114.

116. An animal comprising the protein of claim 113 and/or the nucleic acid of claim 114.


SEQUENCE LISTING (66 sequences)

SEQ ID NO: 1 - protein, 179 aa, Mus musculus
MAVSRITRWRLMSVIFGIKCLFLMVTLGVLLINSFTIQNIQSTPSPTTTVEFQEVSECCVCLDKWVGHQCNCYFISKEEKSWKRSRDFCASQNSSLLQPQSRNELSFMNFSQTFFWIGMHYSEKRNAWLWEDGTVPSKDLFPEFSVIRPEHCIVYSPSKSVSAESCENKNRYICKKLPI

SEQ ID NO: 2 - protein, 179 aa, Homo sapiens
MAVFKTTLWRLISGTLGIICLSLMSTLGILLKNSFTKLSIEPAFTPGPNIELQKDSDCCSCQEKWVGYRCNCYFISSEQKTWNESRHLCASQKSSLLQLQNTDELDFMSSSQQFYWIGLSYSEEHTAWLWENGSALSQYLFPSFETFNTKNCIAYNPNGNALDESCEDKNRYICKQQLI

SEQ ID NO: 3 - DNA, 3601 bp, Artificial Sequence
  5' homologous arm
taacaagcaaaacctactagagtttatgagggtttttcatcaccatgattacaagtgactttatggcaggccagaatcttccttttcaggaagcctccttaatggagtgtgctttgtagcctggtgcacaatgggagggcacagtctttacaagcactgaaaaaaaaataagtatctccattgggatgcttttaccaagctgttgtacagtctaggtgcacatgtctaggtatagagaactctccattgctcacatcttcagtgggcattaaattccctttctcggatctttcctcaatctttcaaagatgtaattattaaagtcaccacttgctttgaacaaattctctttgttcaaagagcagtagacttaagagcaggggaagcagagggaagagatgaacattggaagaatttattattatctgaaacagattcagtgaagctcaatatcccactgagtattatgtgagttatgagatgctcaacaccctatgttctgtgatctcaattccaatgttactgaactgtatcaaatcctgatttatttccttaaaggccagcctggtgataatttgtcagcactgtgtactcccatgtgttggaacatcaaggtttccagaagttttaagagaaaaaaaaaagtaaaggtcatatatataggggtgtatacagtatttgtccagagtttatcagatattcaagagagatgattggggaacatgagatgtgtgttgagtggagatgagtaggagggatggcaaggagtgtaaacaattcatttacctgtgtacaaaatgagagcaaaatcaaggatagctagtagtttagaaagaattaatgggaagattaaaagtgtgtgttggggagagagagatgggaagagagaaggtggggagagggagagagatcagggaagaaagaaaagtgagaaaacacaataatactgaattctttctgggtcttgctgactatggttggggtcagtgatcagggagcagaaacacagaacaagtattggatatcccaacagtttgtactaaagatgggaaatatcaacccagaatgtgcaagtccaaggatgtgcttggtttgagataaattccatgtatgctttggtaaaagactgggcatcaagcttaagtaaccttagtcaaatcagcacttctgtgcctcagtttccccactatcaaatttggtattttctttattaagcgatcagataatatgtgatttaaaatttgtttaacacaaaataaataagtatttctttattttgaaggtagtaattctgtgtgggattttatgataccaagtgagtttaccatggtttatacacctctccattattttctaaactgcttttagggacgactttgatgtgtgcacattgaaaacaatcttcctccaaccttattttgctgtggtgttttgtaccaaaactaaaagagcaaatttcctgtgtgcagtggttcctgggtcttgttcaaagcttgcagtctttcttctgctcattcccactgtgcatctgtgtcccaccaacagatgattcaatagcaccgccacaacacacagtgtttcttcctcttttaatgcttactgtctcctccgtagcttcctgctcagccctgtggtccacaccgttctttaaaaagcacaaacacatctctgcaccatccttggaacatcacttctcatggcaggtatgtgtgcccctgtcagcagatgtaatagtagtgataaaataataaccaggaaggatgcctcttgtattaactaacttcaaggagggtttaaaaaaataaaacagaaagcatataatctgcagtagatatttttagactttctgtgctttgatgctttctgtgttcattgttttctcgattctgtgttttgaaacttgaacatgattttatgaagtgcatacttttgtaaacttttcctaactgagttagcaagtggtgtctattaggcatcatgttaaattcagaataaaacagaaattaaattgatcaacacacaccatatttctaacatggggacaaagatggccaaatataaaggacacttaaaactttataacttttcttgctgtgttgtggccttgcaaaatcactcaggctcactgacactgtgtgctgttgttagcagtagaactatttttgacagtattgcgttactaatacagattcttctcatatcgaaatatcttagccaataggcaagtgattttgaatatgacctaacataatgtttaagaacagaaattcaaaggctggctttgtgtcataattcaaggtgctgttaattgaacccagatcctcaatttatgcttgttatggaacctcctgccataccaggtctagaaggttcctttttattttcctgcagacattaacaaaccactaatcaaaaaatggtctgtgttcaaagggaccccattccatcatggtattctcctgacaaggatggtatataataaataacagactctaggtcttgacaatattctgtttggttttgagtgggcccttactaactttatatcttaagaaaatagcattgtgattactgcccctccccctttaaaaaatcatctctttaacacaaaactttttattcatgacattcttgagagaaaaaaatgattattaagcaagcccaccaagcgtcatgactgcttgtggatctattctaaccatagagataagtttcactgctgatttaaagagtcagaggtaaatgactctaatgaggcagaaaatatgtatgaataacctcatcaaagaacaaggtataccacttcaaaagattgctaatgggtggtcttctgaaggaacatcttcgttctccagtgttccactagagagaggtgtctgcaccactaaaggaagaagttttcaggttcattcttaatggtttctgttcttaagaacgtcatccacaatgaaggagctagagaaagtacccaaggatgggaaggggtctgcaaccctataggaggaacaacaatatgaactaaccagtaaccccagagctcgtgtctctacctgcatatgtagcaaaggatagcctagttggccataaatgggatgagaggtctttggtcttgcaaagatgatatgccccagtatagggtaatgccagggccaggaagtgggagtgggtgggttggggagcagggggaggggaagggtatagggatttttggagaggaaactaggaaaggggatagcatttgaaatgtaaatgaataaaatatctaataaaaaaaaggaaaaaaaagaacgtcatcttctatgtctgcactttcatctgttagtcattaatgtatgtgtttggtatttttttatagtttctaggatcactcggtggagactgatgtctgtcatctttggaataaaatgtcttttcttgatggttactttgggagttttgctgataaattgtaagtgttcctaagcaagtatataggaaaaaaatcaacattgtgattaaataatttaataataaacatttaatttttctaatgtgtaatattttatttcataaatacatgtcaaactaagcaagtctataataatttcttatagcatttact

SEQ ID NO: 4 - DNA, 4488 bp, Artificial Sequence
  3' homologous arm
gatgtggtttgattggttctgttcctcagtgtatatcttttatttgttcttcctgaacaatttctatgattctaagcactatattaaattgtagagatccttgtcccattcctgattttaagtgaaacaatgtcagtttatccccatttgtagtcattttggccataggtttgtcatctattaagacacatattatactaaagtgtgctcctaatatttctgatttatccagggtttgtattatgatgggatgttgaactttgtcaaagtccttttctgcatctatacaattgagaacagtctccccaaagtaatctgtaaaaaatgatagattacttgatttcagtatgtatttgctaagaaagttataacttagtaaattatgcattgtgaccaacttgcaagttcctcaccaatgcagcactgagtcattttcttctgcatcatatccatgtgaataaccatggcaaagcaactgaacagaaagcagagtttattatttaaaaggctagatcatacgtattattaaccattttctatctcctcagttgatctagaaggagccagcgacagtaccattagctgtcctgttaagcagactgtgtaaagaagaaagactgaatgacagtctgcagggattgatatctgcttttgtcatctttgtaatgagcaagctttttttgcattgactttgtagcccagggaaagctttcaaatgatcataccacagtcaaggtcttggtgacaatcttatctcttgaacatttccagtaggggcagggaaatagcacagatagtaattctcttcacacctcattagcatgattctggtggtatcagctaaatataacctggctctaaatattaaaatagaagagtgaactgtttccaattttctgaggaactgccagattgatttccaaagtggttttatcagcttgtaaactcactagcaatggaggagtgttccttttttctccacatcatcaccagcatctgctgttatctgcagtttagatcttagccatccttattggtctgaggtgaaacctaaggtcattttgatttgtatttcccagatgactaaggatgttgaacatttctttacgtgtttctcagccattcgagattcctcagttgaaaattctctgtttagctctgaactccatttttcatagggttatttggttctctggagtttacacacacacacacacacacacacacacacacacacacacacacacacacacacacacacactgtttttgttttatcctattgacagtgttctttgcattacagaagcttttcaattttatgaggtcccatttgttgattgttgatcttagaggagcatgagccattggtgttctcttcaggaaaatttcccctgtgctgatgtgttcgaggttgtttcacattttctcttctattatattcagcatacctggctttatgtggaggtccttgatccacttggacttgcgctttgtataaggagattagagtagatcaatttgcattcttctacatgctgatctccagttgaaccagcacaatttgatgaatattctgtcttttttccaatggattgtttgagcttctttgtcaaagatcataagtgtgtgggttcatttctgggttttcaattctattttgttgatctacctgcctgtctctgtaccaataccatgcagttcttatcactttttctctgtagtacagcttgaggtcaagggttgtaattcccccagaagttcttctattgttgagaatagttattgctcttttggggtttttattattccagatgaatttgacaattgctctttctatctctgtgaagaattgagttggaattttagtgggaattgcatggaatctgcaggttgcttttggtaagatggccatttttactatgttaatcctgacaatccatgagcatgggagatctttccatcttctgaggtcttctttgatttctttcttcagacttgaagttcttgtcatacagatctttcacttgcctgggtaaagtcacaccaaggtattttatattatttgtgactattgagaagggtgttgtttccccaatttctttctcagtccgtttatgagtagaggaaggctactgatttgttagagttatttttatatccagccactttgctgatgttgtttatcagctgtaggaattaattggtagaatttttggggtcacttatgtatactgtcatattatctgtaaatagtgatatcttgacttcctccttcccaacctgaagacccagtgttttagtcagggtttctattcctgcacgaacatcatgaccaagaagcaagttagggaggaaagggtttattcaacttacacttttcacattgctgttaatcaccaaaggatatcaggattggaactcaagcaagtcaggaagcaggagctgatgcagaggccatggagggatgttccttactggcttgcttcccctggcatgctcagcctactctcttatagaacccaagactaccagcccagggatggtcccatccacaaggggcctttcccccttgatcactaattgagaaaatgccttacagctggatctcatggaggcatttcctcaactgaagctcctttctctgtgataactccagcttgtatcaagtttatacataattagccagtacactgagctataccattactgggcatataccaaaaaagatgctccaacatataacaagaacctatgctccactatgttatagcagcctcatgtataatagccacaagctggaaacaactcagatattcttaaacagagaaatggatacaggaaatgtgatacaattacacaatggagtactactcagctcttaaaaacaaagaactcatgaaattctgaggcaaatagatggaactagaaaatatcatcctgagtgaggtaaccaagacacaaaaggacaaacttggtgtgtactcactgataagataatattagctcaaaagatcagaatacctatgatgcaacccacaaaccatatggaacttaagaaggaaaaccaaaatggggatgcttcaatcttacataaaagaaggaacaaaataatcacaggaggtagatggagggagggaccttggagggatagaggaacaggagggaaaaagtgggggaagaaacaagtattaggaggggcagaagtacagaaggccagaaaatgaaatagaaatatatagcagtgggggatggggaaattcactagaaagtcccagactccatggaagcgagagatcccagtactcaactggaatgaatttagccaaaataatgcaacaaatggggagatagaacgtcctgaagacaggcatagctcccagttgagggatggggccacctacccatctcaaaattgttaacccagaaatgttcctgtccaaaggaaagacagggacaaaaaatggaacagagattgaatgaaagaccatccagagattgccccacctagggatccatctcatttgcagacactaaaccctgacactattgccgatgtgaagaagttcttgcttacaggagcctggtatggttatttcctgagaggttctaccagcacctggtcaatacagatgcagatactcacagccaaccatcagactgagcctggacacctgagtggtagaggtcggggaaggactgaaggaaatgaaggggatttcaaccttgttggaagaagaatatcaactggacaacctagagctctcagtgtatacatggagatatttgtggctccagatacacatgtagcagaggatggtcttatctgacatcagtggaaggagagacccttggccctgtggaggattgatgcctcagggaaggggaatgctagaatagtttggtgggagtggatgagtaggtagaggagcaccctcatagagacaaaggggaggggggggagagggaagatgtgatgggacatttgtggaggggtgactaggaaggagaatatcatttgaagtgtaaatgaataaaatgattaataaaagagtggaaaagagaaagtatttgtacagcagagattgcatctaaacaaccagactaagtcagtgataaagctaatgtgaacaatgatacttcattaaattagcagattaactgaatgaaattaatatgcctaaaccaaatagcattgctttacattaatagtaaccagttaggaaaacataagagatgataaatcctcattaacagcaataaaaaagaaaaaatatgtaaaaatgaaatttaaaaacatatcagaaacatatgtaaaggaactactgaaagatacaaaatgagacaagaatataaaggcatttaattttgaaactttttaattaactttttttaaaaattaaactgttctctttaaaaacctagtgatatatttagtggagctgagcaatttgacagagataatatgtaaatgtagcctcagtgttta

SEQ ID NO: 5 - DNA, 5157 bp, Artificial Sequence
  synthesized sequence
aaactgagtattgagccagcatttactccaggacccaacatagaactccagaaaggtaggtcacattttttggaaaacttagcattggtaaaagattaaataggcagtttcttgtttttcacacagagaggcatgatggaatattatacttagtaatagaaatttgcctaccagtgtaggatagtctcattttacattgctcaatctaatgtaaaaatgattaaattacactgaatgtgtatcatttgtacatttaactaatcaggaaaatagtagcctgaaacgaaagatatgtttcctatatgagtgctcataattaagtaacaaaaaataaaccacaggaaatgttacagttaatcctcattacctgcctgaatttgcttataacctatacttttgcatctcactacttttccttttccagaatatactgcacagatttctaaaccttacttatccagtgaaagccagctcaaagtccaattttcgtttttggagatggagttttgctcttgtcgtccaggctggagtgcagtggcacgatcttggctcactgccacatcagcctccctggttcaagtgattctcccaccttcctgcctcagcctcccaagtagctgggactacaggcgcccgccaccatgcccagctaatttttgtgttattagtagagtcagggtttcgccatgttggccaggctggtctcgaactcctgacctcaggtcatccacctgcctcggcttccaaagtgttgggattatgccgtacccagcagagccactgcgcccggcctcaattttctaaatatgtatttttcggaatgcgccccaagttgtctcaccatcatcttgattatatagtgtttatttacagtgcaacatcttggccaggcacagtggctcacgtctgtaatcccagctctttgggaggctgaggtaggcatattacttgaggtcagaagttcaagacaggccaaccccgtctgtactaaaaatacaaaaattagccaggcatggcggcacgcacctgtagtatcagttactcaggaggctgaggcatgagaatcacttgaacctgggaggcggaggttgaagggagctgagatggtgccactgcactccagcctgggcaacacagcaagactctgtctcaaaaacaattaaaaaatataaaaataatagtgcaacatcttcagcactacttatatggcaaagatttgagtgcctatgacgtgtcatgtttttattaccgtgggtaacaaacgttgaagacactattattgataaactgtataattaagcctcaagataattggtttatttatattcccttttcaaaaataagatatccaaacatatatcaaaattctccaaatgtaccatattcttttctgactcagaaagtatgctaaaatgttttcctcctttaaatctttttcatctagttaactcctatttatgctacagtacttagttttgagtcacttcctcaaaaaaaggttcttgtaactgcccctcatcttacactctgctacatgctgttttaacatacttaatttttttgtacttatctaaatcatcactaaatatcgttaataaattattgaaaattatataattgtaattctatctctagaatataatctccatgaggaaaaagcatcatgttcatgtctaattagttcacattagtagcccctgaagtgtggtgtatacagtagattctgaatgttaaaagaatacacagcaaataatttttgaataattaaatagcattgaaaaaaatgtctagtctccagtgtcattagtcatgctttatgttttctgtcagactctgactgctgttcttgccaagaaaaatgggttgggtaccggtgcaactgttacttcatttccagtgaacagaaaacttggaacgaaagtcggcatctctgtgcttctcagaaatccagcctgcttcagcttcaaaacacagatgaactggcatgtgctgagtctgattttctacattttctttgatctagaaaaatatactatctaaacaagttaaatactttggtttaagtcattaatcacatagtaagtggatggttatattggaattgagttatctgttctgtgtaccttaaagtagtcattgtaaaatctttattaaattccttaatgattatatcatgggaatacaaatcaagataggtgccaaataaagtgaaagaaacttttcaggaagaattcaaagaagataataggtcattatatttacccattttatttgaagaaccaactaaatgactgtgattcttaactcttttaattgaatttataatatcttttataattttatagtttttgtcatctttctgggatataagctagtatggaaatatggtaagaaaacgtacccaattcttttcttttctttttttttttttttttttgagacagagttttactctgttgcccaggctggagtgcactggtgcaatttcagctcactgcagcctccacctcctgggttcaagtgattctcctgcctcagcctcctgagtagctgggattacagacacctgccaccaggcctggcactttttgtatttttagtagagacagggtttcaccgtattggcgaggctggtcttgaactcctgacctcgggtgatctaccctccttggcctcccaaagtgctaggattacaggcatgagccaccgtgcctggccctaattacccaattcttttttgtttgtttgtttgtttgtttgtttgagatggagtctcgctctgtcgcccaggctggagtgcagtggcgtgatcttggctcactgcaagctcctcctcccgggttcacgccattctcctgcctcagcctcccgagtagctgggactacaggcgcccacaaccacgcccagttaattttgtgtatttttagtagagacgcggtttcaccgtgttagccaggatggtctcgatcttctgacctcatgatctgcccacctctgcctcccaaagtgctgggattacaggcgtgagccaccgtgcccggcccctaattacccagttcttaagaaaaagtgcggccgggcgcagtggctgaaacctgtaatcccagcactttgggaggccgaggagggcggatcacagtcaggagatcgagaccatcctggctaacacggtgaaaccacgtctctactaaaaatacaaaaaattagccgggcgtggtggcgggcgcctgtagtcccagctactcgggaggcggaggcaggagaatggcgtgaacctgggaggcagagcttgcagtgagctgagatcgtcccactgcactccagcctgggcgacagagcaagactccctctcaacaaaaagaaaaaagaaaaagcgaaaaacttctcctgctgcacgtggcacctttgcggattataggttgagtagattatacccatgggattcctccttcacaataccatgattttggaatcaaatttaatctttgatcccccgtgaagaaagtgttccatgaaaactctatctccttttagaaggtttttaattcttcaggaagtgagattttgtggtattttttctccatatatgaatgggcctgataaatgagaactttcagtcttgtgattataccttttaaataggaagttaaaaataatttccgtttgtgtgatggacaggctgaatcttattctaaacactgactttccctcaaaatatgttattcccagaatagattaaaattagattaatgtgattgtcttttacttgaagcaggattttatgagctccagtcaacaattttactggattggactctcttacagtgaggagcacaccgcctggttgtgggagaatggctctgcactctcccagtatctgtaagtttctggcaatcatggcgttttttgctctttatgaatttgtccttaaatgtgactagtaaaggtaacatccaggagcactgtggctgaaataaccttgtctagggcatgagaatacagtaaaaggaaaaagtacaacaaaataacaattttagatctaggcagatctaaaccactctaagtgttaaaatcaaaacagaagataatgcctaatgaaaggaggtcggggtataggatagttggtgcaaaaaaatactgataaccattacatgcttgcctgtattattttaaattttttaattaaaaatatattattttgtagtcacatggacaacaaaaccaaaacattacaaatctccagaaagtgcaatgcataagctctcttagttcaaattaaacatttgaaaggtattatatttcatcctatatcttttgtcagttaactgaagtgcatttttcctaaatcaatcttcatacaagaatgtgaagagggccagggatggtggcttgagcatccaagttaatgctcaagtgacatgaaatttagtgtattcgtggccataaaagaacttttaacatatgctttatttttgccttagtaccagcacttggaagtatatgtgcatgtacagatactcatatacacagaggcagtccaaaaaaatagtatatttattattttatttattctggtagaccccaatattctcagcatgctcttatgttttatatactatagactcattgtctgtatgtgtgtactttttgaatctcattttcatttataaacatgagatttgcagcatcacacagggacaaagtttatgctctggtttgttttcctagactagatcagataatacatcttaaataaaatagaagaaaagcatatcacatatttaaaccattaatgacaacagaaaaacagcaaggaaaaaatccctggcctcaatgctattgattagtgagaaaataactgggtattttgtagcattacactagaaaatcatgaaaattgtggttactgaaaaaaaaaggactcaatatgttagtatctcactcaaatgcttttaaaatttatatcatttaattgaaaaatgccctgaacattcttacttcctttttgtgtatgtgaacattttcttcttcattacagatttccatcatttgaaacttttaatacaaagaactgcatagcgtataatccaaatggaaatgctttagatgaatcctgtgaagataaaaatcgttatatctgtaagcaacagctcatt

SEQ ID NO: 6 - DNA, 80 bp, Artificial Sequence
  synthesized sequence
gaagataaaaatcgttatatctgtaagcaacagctcatttaaatgtttcttaaggcaaagggtatagacaaggaaggtcc

SEQ ID NO: 7 - DNA, 867 bp, Artificial Sequence
  CD94 mRNA sequence of the modified humanized mouse
atccttggaacatcacttctcatggcagtttctaggatcactcggtggagactgatgtctgtcatctttggaataaaatgtcttttcttgatggttactttgggagttttgctgataaattcatttactaaactgagtattgagccagcatttactccaggacccaacatagaactccagaaagactctgactgctgttcttgccaagaaaaatgggttgggtaccggtgcaactgttacttcatttccagtgaacagaaaacttggaacgaaagtcggcatctctgtgcttctcagaaatccagcctgcttcagcttcaaaacacagatgaactggattttatgagctccagtcaacaattttactggattggactctcttacagtgaggagcacaccgcctggttgtgggagaatggctctgcactctcccagtatctatttccatcatttgaaacttttaatacaaagaactgcatagcgtataatccaaatggaaatgctttagatgaatcctgtgaagataaaaatcgttatatctgtaagcaacagctcatttaaatgtttcttaaggcaaagggtatagacaaggaaggtccacggttactaaaaatagtaacagctctcaatattactacctttccatcttgtgtctgtagaatgtttttgtttggtagtatataattctcatacatttgtacccaagtaatttatacttgatggcttcttcacacgtgtgcataaagttcagatgaagcaaaaatagagaggttttataaatattttgacttatgaaatataattttattcaatattcctgtaaagttttcttggaaacattaaataaaatttattctacaccaagtc

SEQ ID NO: 8 - protein, 179 aa, Artificial Sequence
  CD94 protein expressed by the modified humanized mouse
MAVSRITRWRLMSVIFGIKCLFLMVTLGVLLINSFTKLSIEPAFTPGPNIELQKDSDCCSCQEKWVGYRCNCYFISSEQKTWNESRHLCASQKSSLLQLQNTDELDFMSSSQQFYWIGLSYSEEHTAWLWENGSALSQYLFPSFETFNTKNCIAYNPNGNALDESCEDKNRYICKQQLI

SEQ ID NO: 9 - DNA, 80 bp, Artificial Sequence
  synthesized sequence
aagtatggtaacatatcatctgcggatgaagcttgatatcgaattccgaagttcctattctctagaaagtataggaactt

SEQ ID NO: 10 - DNA, 85 bp, Artificial Sequence
  synthesized sequence
tattctctagaaagtataggaacttcatcagtcaggtacataatggtggatccaggcctgatgtggtttgattggttctgttcct

SEQ ID NO: 11 - DNA, 24 bp, Artificial Sequence
  PCR primer CD94 F1
ttcctggcaagaaatgatactcca

SEQ ID NO: 12 - DNA, 24 bp, Artificial Sequence
  PCR primer CD94 R1
atgcctctctgtgtgaaaaacaag

SEQ ID NO: 13 - DNA, 20 bp, Artificial Sequence
  PCR primer CD94 F2
caggacatagcgttggctac

SEQ ID NO: 14 - DNA, 23 bp, Artificial Sequence
  PCR primer CD94 R2
ggaagagccatcactgttaaggt

SEQ ID NO: 15 - DNA, 24 bp, Artificial Sequence
  CD94-5' probe F
acaagccaaacactaaattggcat

SEQ ID NO: 16 - DNA, 22 bp, Artificial Sequence
  CD94-5' probe R
gtgggccaagtagacacttcct

SEQ ID NO: 17 - DNA, 25 bp, Artificial Sequence
  CD94-3 probe F
cacaacattaagttttccctctagt

SEQ ID NO: 18 - DNA, 25 bp, Artificial Sequence
  CD94-3' probe R
gataatccagtactgccttgatagt

SEQ ID NO: 19 - DNA, 23 bp, Artificial Sequence
  Neo probe F
ggatcggccattgaacaagatgg

SEQ ID NO: 20 - DNA, 23 bp, Artificial Sequence
  Neo probe R
cagaagaactcgtcaagaaggcg

SEQ ID NO: 21 - DNA, 23 bp, Artificial Sequence
  CD94 gene detection primer WT-F
tagtttctaggatcactcggtgg

SEQ ID NO: 22 - DNA, 24 bp, Artificial Sequence
  CD94 gene detection primer WT-R
tgctgtagaatgagcttctctgtt

SEQ ID NO: 23 - DNA, 23 bp, Artificial Sequence
  CD94 gene detection primer Mut-R
attccatcatgcctctctgtgtg

SEQ ID NO: 24 - DNA, 21 bp, Artificial Sequence
  hCD94-RT-PCR-F2
ccagcatttactccaggaccc

SEQ ID NO: 25 - DNA, 20 bp, Artificial Sequence
  hCD94-RT-PCR-R2
ggagagtgcagagccattct

SEQ ID NO: 26 - DNA, 22 bp, Artificial Sequence
  mCD94-RT-PCR-F1
agtttctaggatcactcggtgg

SEQ ID NO: 27 - DNA, 20 bp, Artificial Sequence
  mCD94-RT-PCR-R1
tgcagtgctctggcctgata

SEQ ID NO: 28 - protein, 244 aa, Mus musculus
MSNERVTYAELKVAKNSRNQHRKPRGPRSSISVIEQEIIYSDFSFQNPSQEHPWICRNCPCKGFPSPPEKLIAGTLGLICFVLIVAVVVITTVATPYTEAKAQINSSMTRTHRDINYTLSSAQPCPHCPKEWISYSHNCYFIGMERKSWNDSLVSCISKNCSLLYIDSEEEQDFLQSLSLISWTGILRKGRGQPWVWKEDSIFKPKIAEILHDECNCAMMSASGLTADNCTTLHPYLCKCKFPI

SEQ ID NO: 29 - protein, 233 aa, Homo sapiens
MDNQGVIYSDLNLPPNPKRQQRKPKGNKSSILATEQEITYAELNLQKASQDFQGNDKTYHCKDLPSAPEKLIVGILGIICLILMASVVTIVVIPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 30 - DNA, 4052 bp, Artificial Sequence
  5' homologous arm
ttggaggctggaagaatcagattccaccccagaccccagcttccaactggagttactgatggttctgacttgtcatgtaagtgttgaacacatgtcctctgaaaaagcagccagggctgttaactgctgattcttaccccttcaaaatgtgagaatgttccttgaattgggtaggggcttcatagagcctggcttggtttttttttttttttttttttttttttttggttttgtttgttttgctttttttgttgttgttgttgttgttgtctctgtttttgtttgcttgcttgcttgcttacttgcttgttttgacagaactgtgctgcttcttgccagtgtacctattgcacatccagccacaaaccctctaacttttctgtattcccaacctcttcattttaaccaataaaactgtgttacctactccttctttatgatgttatttttaaaggcacttttttcagtttctgacttctaaattaaaatgctactgagtttttattcaatccctaaattttaaaaactgaatttgcttatttccttctaagtgttttgttactgtgtttaggcttccatgccaagattatgtaatgtcaaaaataggtgatttaatttaaatcctcctgatagtgacagttttgtcagagaatcctataaattactattttaattatgaaataaaacataagggtgttgggaacatatcttaattgttaagagcacactctgatcatccagaggtttcatgttcaatccccaggacccacatggtggtatacattcttctatagctccagtttaaggagatccaatgccattttctgccctccaagaacactaagcatagacatggtacagagacatacatgaagaaacttctatacacaatgaaacaccttcacacataaaattaaataaatgattaattataagatctcaaacataaacataggagtttgaaaacatgagtgtgcagtggtgtcttctgtgccagccataagagtttgaaaatatgctcacttctcaggaatatcagtaacaaatagcactatacatgtatgtatttataatattcctatgattttaaaatttttccatcatggtatagttaaaatttaagatatagtatctatctatctctctaaataaatatgtctctgtgtgtattataatgttggatatagatatagattgtgtgtgtgtgtgtgtgtgttataatgttatattcacttgacacaagaagtctaaggctgaagctcttgtaaaattaggaaagaagagagagaggatggggatgaagggaacagaaaacaaaccagtatactagtgtaaaatcaaggcttacaaatcttaagtttctcttctatcaggataattattatgattgcggtagaagacaattaatttcatgtatggccacttttctagccccaatggctttatcttatacatatacaattttagggaaacagttggaaaagaccacactgtgcaaaacactgagagacactttgtaccttccacaagaacccaaagcattctctgctgtaaaggtgtgtgatgaaaaaactgatttcaaaatcagcttctctgagtatgagaatttcctgttcaaccttcaatgacacattagtgagaaatctccttctccacacttgactcactctgaggctcatggggcaaccttcaaagttcacagtcactgctgccctagatttctgccacagacaaaccctttggtgcttggctctcacagactgcctgtgacaaccacacctcactgcaggaagcctggctttctgccaatacagggagcttatcactgcttccttctctccacctgcccagtcagttaagggaacactgcaaatgctacatgtggagcaactgaggcaccgttcagatgcagaggaacaaactggaacatagatgcctttaaacagctcagataaagccaagtgcatggaacacacaggggaccgtctcaaagtgcttgtcatcgtctatggagagttagccatcatctggaacacactggggaccatctcatagttctgtccttgttgtctatggagagttagtcaccctcttgcatttctgttcctttatgaatataattaaacctactactacggaggacctgatgatgaagtgtgaattctacgtgttgaaatctatgtgctgtggctctttggatgaaaccatatcaatttatcccagagggtgcagattcttaaaaggattctctcactgagtggtgaagaacaatagtgaaagtggaaactaaaatattgatttaaataggtagtatttattgagagctctgcatgccagaggggagtactaattgccttacatgatttttttttcattttagattaatgcacagggctattactaacttctcaagagtcacacaagttttaaaaccagaattaattctagatctcacttattccagggctaactgattgaattgttaaattatattgggttattacaattgtattttatgtgactaaaatgacactcaatttggcagccttggactctttgggaaattttgaatcatcattctactatgaatcattttttcatgttatagtttcacttgttggctaatttttttaaataaaatggaattaaaatggtctttgaaatattttgaatatcatttgttttaagcatgatttttttgacatagcattttcaagaatgtacaatatcctttaagttttagtcatttctctgaactcaattattccaaataaaataaattttacatcaaacatattttaataaatacattagctattaaaatcatatgcatgttttatacacatatatatgtatcaaccttcctaggttacctatctagagaagtcagattttctcattattggattttaatttattactaaagtttagctgattgctgtgtgtgcatgcacagataaaaatatagcaatgataattaaagaggagatcatgaatttcagagagggaggaaggaatgaaacaggttggaagggaagagagggggatagaaattatgtgaaattctgaaaataattttaaaaagtaatttgaaatgaattcaacattcatttctattagtgttgggatctttggtgtaagcattacctatttcatggtgttagtcctatgaaaactaaatttaatttataaaacaattctttggtttgtcagcatctttgttccccattaagttgttgccttatcaaaaatattttgccaaaagttgttgttgctgttgttgttgttgttaccacagcactcgtattcattgtccaagggaagaaaaatagattaggtttaaatttccaactgcaaaatgagtgccattgttccaaactcctcctcctttggcctcatcagattgtgtacctttgcctcagcacatcactagctgcagacatgagtaatgaacgcgtcacctatgcagaactgaaggtggcaaagaactcaaggaaccagcacaggaaaccaaggggtcctcgcagctccatttcagtcatcgagcaggaaatcatctattcggacttcagctttcaaaatccttctcaggagcatccctggatctgcaggaattgcccctgcaaaggtgagccactgggtggaccctgcactgcgatgccagggccatgaagaggtgtgggcagaggtagaaatggtcttttttgggagggccgtagttctcacttgacctatgcttttgtaaattgggagcgtagatttcactctgttgtaagactttctggatcgctctgttcaccaaaagtaaaacagtactgcctacaaacagatcactgcatggagaagagccggggatcagaggttatctgtttgtctcaatgtgtatgtaggttttttgtgttcataaaccttgtgaacagccaccttcataaacccttgcatttctctctgaaggttttccatctcctccagagaaactcattgctggtaccctgggcctcatctgttttgtcttaattgtcgctgtggttgtaattactacagttgccaca

SEQ ID NO: 31 - DNA, 4631 bp, Artificial Sequence
  3' homologous arm
acccacttttagtcaataagtaatattatatataaataattaatgtaaaatatttaatataaaataattaatataaaaaattatatttttgtatctatacataaacatatacaactacttctgtgaatatgtatgtgtgtatacatgaaaatgtgttctaagactttttatccactagatacactgagaagttttattaacatagtttcagtatctaaggtctgtaaaacttataaaactttttaaaatatagacatcaaatatttacaccttaatactttttcaggggcctttgttcaggtgcgtgggtgtattgtttcttctgggttttttcactctctttcacacatcatttacttatatctttttgttttaatgtgtgtgtgtgtgtgtgtgtgtgtgtgtgtgtgtgtgtgtgtgtgcgcgcgtgcgcataaatgcttccatggagcctgtacaaagttgaggacaacttacagaaggtggctccctcctaacattcagggatcaaactgagattagtatgtgtgatagcaaacagtgttacctgatgaacaatctccaaaccctttgttttgttttgctttggtttgtttgagatgagctctctctatgcagcacagaatgacatcaaactgtcagcagttattttgtctcttggagcagaggcttacaagcctttgttgccaatggaggccttcttctctcttttcaattaaagggactttggtctttattgaacttttctgttggaatatttttcttgattataattttgtgtagtttattgctttctgctttttgcttcatttgttgtgaacttcaattatttagccttcctgattttaatttcttttaatacatgttcatatcaaaatatcttttcattttttcttttttaattaattaattaatttttttacactccatattttatcgctcccctgatccaccctcagattggtccacatcccatacatcctctctacccacctgtctccacgtgtatgtccccaccgccactccacctgacccataaactccctggggcctccagtctcttgagggttaagtgcatcatctctgaatgaacacagaccctgcaatcatatactgtgtgtgtgttgaaggcctcatatcagccgatgaatgctgcctgtttggtggtccaatcttttacagatcttggaggtccagattagttgagtttgctggtcctacaggattgcccttctcctcagcttctttcagctttcgctaattcaacaacaggtgtcagatgcttctgtccattggttgggtacaaatatctgcatctgactctttcagatgcttgttgggtctttcggagggcagtcatgctaggtccctttttgtgagcactccatagcctcagtaacagtgccaggccttgggtcctccccttgagatggatcccacttcaggcctgtcagtggaccttcttttcctcaggtttctttccatttccatccctgtaatttcaaagaggaacgattaaggctcagagttgtgactgtaggatggcatattaaaaatatctctttttctgtaataatacctttgaagaaatcaattattcgtgagggaaattttgctatgttttccttataatttttaattgtgaggatatttaatgataatatgtttgtcctattattatttaaaaatatgctacagaattagatgatacatacttcaaaatgttggccatttattgtttgttctttttttttgataaaccaaaaggaatttttagtgatgcttccatgtaactgtgaatatggctactaagactgaaaagtttactttgcaacttattttatagtttattttccttttaattaagtttacacatgaatagctacaatttgaacatcttatgtaagcatggccatggctattctatggatagatcagtttaaatactttctaattatatgccgagaaatatttgcatatttctgtgttaatttcccatgtgtatctaagaacctaagccttctgtgctcacagaagtgagtatgtaagtgggagccagtgttaactctccagcttaaatgttttcataacctcacaaattcatctgctggcagaggtgtggtgagatatatctaggtcccattcaggattaaactcttgtcttccaattctatgttttgttttgttttattatttctttacttttatgtatataaaagctgatgaaatattattactggtttgaatgacaatcttattattcaacattcttctttatctccaataatgatttctgtcatcatatgagagtacactattggttcatttttattttgttactttcatctttccttatgcctatgttgagaggtttatattcatatgcaaacaagacaagaccatgttatttaagttaaatattatttagctcatatttaatgtattattagattattgtaagatgttatttctaagttgcttgcatatataattaattctaatcttcacttaattttgaacacttctatcccataaactctactttttcttcttccttatcttctcacaatatttttggtgatcacattccattacataaatttattattttaacttcaatttgtttttttttctttctttccattttttattaggtatttagctcatttacatttccaatgcaataccaaaagtcccccatacccacccacccccactcccctacccgcccactccccatttttggccctggtgttcccctgttctggggcatataaagtttgcgtgtccaatgggcctctctttccagtgatggccgactaggccatcttttgatacatatgcagctagagtcaagagctccggggtactggttagttcataatgttgatccaccgatagggttgcagatccctttagctccttgggtactttctctagctcctccattgggagccctgtgatccatcctttagctgactgtgagcatccacttctgtgtttgctaggccccggcatagtctcacaagagacagctacatctgggtcctttcgataaaatcttgctattgtatgcaatggtgtcagcgtttggatgctgattatggggtggatccctggatatggcagtctctacatggtccatcctttcatctcagctccaaactttgtctctgtaactccttccaagggtgttttgttcccacttctaaggaggggcatagtgtccacacttcagtcttcatttttcttgagtttcatgtgtttaggaaattgtatcttatatcttgggtatcctaggttttgggctaatatccacttatcagtgagtacatattgtgtgagttcctttgtgaatgtgttacctcactcaggatgatgccctccaggtccatccatttggctaggaatttcataaattcgttctttttaatagctgagtagtactccattgtgtagatgtaccacattttctgtatccattcctctgttgagggccatctgggttctttccagcttctggctattataaataaggcttctatgaacatagtggagcatgtgtccttcttacctgttggggcatcttaacttcaatttgttaatttaactttaagtattcatgtctcacatcttagactataattctttactccttaaaattcaataaatcagtaatttcactctttttcaaacaaacaaaagttccatcaatcttcaacttttactcactatccattcaagtttttattctgaaatgctatgatgtaattcctggatactaaagtacatttcagtttgataattcacatcaatattgcactcattcaatatctatttaagaaaatttgaataattccttctgctcttctttttgcatagaattctctcatttaaaagaagcattaatcattaaatgttaaagtgtttcctgtagtcttagtcactaattttgctacttcttggaaaatgaactaattttactatcatagtaaaaaataaataaataaataagaccaatatacttccttttcacttccaattattcatctttaagtgtcaatattccttcaataagagccaaaaagttaaattcacagtagtgtccttagagatgactaatttggaaaaattaaactacccttttcaaaagttagttttatatacatttaataaagtacaaattctaactattcctattatattaaatcccttcactttgtttttcaatttttgttgtattattgtctgaatgaagatttaatagcattaaatgcccaaagagtgaaatcccacaatgttatatataataaaattatgttctgtataacagataaaggattatgtaatttaaaactgtaagtcaatgtctatactgggaataaatataattacatcaaataagaaaaagaaacgttaatgaatagatcatggtttaccttatttgcaagctaaatcttgaagtgcactaatacgtttgtttaacaaaataaatttagttgtaataaattgaatatacataaagtccatatatggaacacttaaaataagatctaatttataaaaataaaatataatgaatagacatgtaggtggacttattgtacaaggcatgtaagtccacacactca

SEQ ID NO: 32 - DNA, 3934 bp, Artificial Sequence
  synthesized sequence
ccctgtaagtctattttcgaagattacaaggggaattttcacgttaatgattgaatgtgcctctaaacatttcatattttcagggaatagagttctcattgtaatgtatatatttggactaaatgtggaatgattattctgaatttgtcaaagaataaatgaaagaataattgttgaaagtattcgcttctgatgcaatcgtatgtatatatttggatttcataactcaaaaatatgttctaggagtctgaaaaaccttactgagaaatagaaattaatttttgaaagtagttaaatcaagaattataagaactatatgagatggtgaaatttggttctttagatctatgaaatacttttccaaaaaaccaccattactttatcaaatttttcttaaaatcaattttattccatattattctaactctaaataatacaaaaaaattcaaacaaaaacttaaaattattatgattgatttagatgctaatttttttacaaagattactttaatttttctagctacattaatacagaggcacaacaattcttccctgaatacaagaactcagaaaggtatataataatttttaaagttttaatattgtacagtttattttttcttgatcttaggctgtacaaaaataaaattttgggtgaaaaattataaaatttggcaataagtgttcataaataaaattagagtaaggcattcacttgtcatcaactataagtaaaatcactatgctttctttttatctgttgtgttcaaattcttactgctataatatgataagatacaagttatttattgttccttaaaaatcagattagttcattgatttttcaagacatgtatagagtggatttttgtttgctggtttgctttatatgggaacacaattaggagatgaaaggctgaccctttattgcgcatgtgtgtataagtgactgggtattttgacactatatatttaccagcccatgaagatgtatagatatgttgcatacgtataggtttatatgtttgcaaatatgtgaactaattttcatttttaaaaattcatattggtctagatagtaattcatatctttatttagcacgtcattgtggccattgtcctgaggagtggattacatattccaacagttgttactacattggtaaggaaagaagaacttgggaagagagtttgctggcctgtacttcgaagaactccagtctgctttctatagataatgaagaagaaatggtaagatgtaaatgttttaaacactttatgaaaagcttctttcggtcgataatatatttgtagaaatcatccatatgtgtgggtatatacatttagcttatatgttttcaagtttatgtagtatttaattgattgacttaataatgttttaaaattcatatactgctaatgtacttttgattattttcagtttttgcttttcatggaaaaccatgcttctagaaatgctttcaatccacaatacattttgctatctaattttatcgggcatgatgtgatctggtcatgcagattgatcacaaagtgaatgaactcctgtgatacaagtcagatcatgaaataaaagtttccagctctagtagttccacccctgtgtatgccctcatcacttatcctgactcctctccaaaacactgtcttgacttttaatgttataaataacgtttacctgcttttgaatttatataaagggaatcatacactgtgaatttcatgtctgtgtttttcactcctatctgatatttatgcaattcctccatattattgcggttatctgtcgtttattactgttcactgctgtagtatgtacaaagaacactaagaatccattcaatcctgtgtctctggatggggaagtgagtctcatgccctcagggacaaagaggaccctgggtggtgcactggtagtcattgggttcctttgctgatcctcctcacccacatccactctggtgtctcttggtatgagaaggaagtacttcctctggctgtattggtagcaagtctcctggtagatcatccttgccagtggtaccagccttgcctgttgttgcggaggggactccccttcaatacagaacaggagtgagctttgctgggcctcctcctattgccaggttgggtgtagggaaacagcaggcctaggtcaccttcttctgtcgtgtggaggacttaacatgctcgctcggacacttggttgatccctgatgctagggtcccagacaatttcatctttctctttccacctttcagagttctccattgcttttgtctttcattaatcccagagtttatagttgtttttagtagggagtagcagagagagacgagtctacaccacctggtcaggatcactgttattccaccaaaaccaaatcagataaaaaagtgagggcttatctagttaaagaatggtgtggtacccagaaaacccaatatgtagcttccatgtcatttatttctgaatcacaacctctaatttctcttctaaatctccaactctgagaaatatagcacaaaaatagtttgatttagtcacagtatctggaggaatgaatgcacagtatcaggagacttatttaaatccttactgtgtttattcagtcaattggggtaactattataatgcaagaattaaaacttctttattaacatgagaagaataaaagtactaagtataaacattgacgggttcatttatatcaaaattataaacatttatgaaagtttttggcactgcaaatagtggttttcaaatttaatatattgtttttgtaatgttttcataattattatttaagtgaaaattctttcttttcttttagaaatttctgtccatcatttcaccatcctcatggattggtgtgtttcgtaacagcagtcatcatccatgggtgacaatgaatggtttggctttcaaacatgagtaagttgttttgtatggcgctatataacaatatatatataaaggataaattcagaagaataatatgaataaatctatgtggaatcatagagatgaagaaagatgtggaaagttagtgaaatgctgacataaatattttacaatagaccatagtagtccatatatttcaaccgctcattggtcagctagtaaccttcttgattatcaggtgtccaatctttggcttctgtgggccacattggaagaagaagagtcttgggccacacataaaatacactaacactaacgatagctgacgagcaaaaaaaagaaaaaaaaaatcacagaatgttttaagaaagtttacgtatatgtattgggccgcatttgaagctgtcctgggtcacatgcggcccgtgggcagcgagttggacaagctcgagctggactatcagggaattgcagtgcttgtttttattaaaaagccacacttactttttttcttaagaatatcctcaaagcacaagagtagtgctgttggcatattgctataattttgttattagtagttattgttgtcaatctcttattgtgcctaatttataaattaaattttatcacagttatgaatgtgtagagaaaacataatctctctataggttctgcactatctgccgtttcaggcatccactggaatcttgaaacatatccctcgtggatgaagggggactactctgttgagtgttcagaataatgactcttactaatattatgaaaaatttaattacccttttccatgaaattattttcttacagtacatggaaaatgctttcgtctcatgaatcatttgcttaaaatgtaacagaatatggatttttctccattacaggataaaagactcagataatgctgaacttaactgtgcagtgctacaagtaaatcgacttaaatcagcccagtgtggatcttcaataatatatcattgtaagcataagctt

SEQ ID NO: 33 - DNA, 47 bp, Artificial Sequence
  synthesized sequence
tcaataatatatcattgtaagcataagctttgaaacacctgcactgg

SEQ ID NO: 34 - DNA, 1966 bp, Artificial Sequence
  The mRNA sequence of the engineered mouse NKG2A after humanization
ctcctcctcctttggcctcatcagattgtgtacctttgcctcagcacatcactagctgcagacatgagtaatgaacgcgtcacctatgcagaactgaaggtggcaaagaactcaaggaaccagcacaggaaaccaaggggtcctcgcagctccatttcagtcatcgagcaggaaatcatctattcggacttcagctttcaaaatccttctcaggagcatccctggatctgcaggaattgcccctgcaaaggttttccatctcctccagagaaactcattgctggtaccctgggcctcatctgttttgtcttaattgtcgctgtggttgtaattactacagttgccacaccctctacattaatacagaggcacaacaattcttccctgaatacaagaactcagaaagcacgtcattgtggccattgtcctgaggagtggattacatattccaacagttgttactacattggtaaggaaagaagaacttgggaagagagtttgctggcctgtacttcgaagaactccagtctgctttctatagataatgaagaagaaatgaaatttctgtccatcatttcaccatcctcatggattggtgtgtttcgtaacagcagtcatcatccatgggtgacaatgaatggtttggctttcaaacatgagataaaagactcagataatgctgaacttaactgtgcagtgctacaagtaaatcgacttaaatcagcccagtgtggatcttcaataatatatcattgtaagcataagctttagaacacctgcactggaagtctgccagatctctggctccctttagatgaatttgcaactagaatccaaaggatcatcagctttctctaactgtcctgttcgacggtaacaaacacagttattgttacctataagaatgccttttgaaagattatggggaatttttcattcagaaggatggaagtctctgtacattcttctctgaacaaaatcatcagagaacacatttaatatattcctatttatgtaattttgttaaaaatctttccaatgtttattcttaattttgtcaaaagtacaataacaaaattattgcaaagtggtttttagtctatatgctgtgattttgtaactcaatgtcatgctggaaatatttcttaagatgctaattagtatttataagtggttataataattattgtgaaatttggtctgttttagttgtgagcctagcctttaacagctgagtcatctatccagtccaaattttggtcagttttgaatctttttccacataaaatagttttattaaatactttaatattttaatataaaataaatttcaaactaagaaattaacatttgattttcaggaatattaattgtttaagatctcctttatctttttctagctattttaactaaggagcagaacattaaaccctaataaaccccaaaaaggtacacaataattttcaaagttccagtgtttgcacaggtttaactttgtcttgatctcagcctaggcaaaagtgattgtgactttcaaaggaaatgaaccaaagaatcagtcaatgaatcttcacaaatattttacaaaaacatgtattcttgtacaatattcaaagaacatgcttaaggtcaaactgtccattggtatcaatcacctcaaaatttgctactgttgttataagacaagatttaaatactttttcctgtttcacagaatgacttctgtgcagagactatttagaacatgtataggatgagattttgcttgttttcttcatatgcacatagatttggggatgagtgcctgactttttctatgtatatatttattagtgtgtgggctgttttatttgtaacaggctacaagaatgcatgtctataatttgtatgtgcctgattatacctgtgcaaagatacgaagtcattattaaataccaatgctgcttctgagacaaaaaaaaaaaaa

SEQ ID NO: 35 - protein, 235 aa, Artificial Sequence
  The encoded protein sequence of the engineered mouse NKG2A after humanization
MSNERVTYAELKVAKNSRNQHRKPRGPRSSISVIEQEIIYSDFSFQNPSQEHPWICRNCPCKGFPSPPEKLIAGTLGLICFVLIVAVVVITTVATPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 36 - DNA, 80 bp, Artificial Sequence
  synthesized sequence
attgccagttgtatattgcaacttcagcttctgtagtacatttgggtcgaattccgaagttcctattctctagaaagtat

SEQ ID NO: 37 - DNA, 70 bp, Artificial Sequence
  synthesized sequence
aggaacttcatcagtcaggtacataattaggtggatccacccacttttagtcaataagtaatattatata

SEQ ID NO: 38 - DNA, 25 bp, Artificial Sequence
  PCR primers NKG2A F1
atgccagaggggagtactaattgcc

SEQ ID NO: 39 - DNA, 25 bp, Artificial Sequence
  PCR primers NKG2A R1
tgggttttctgggtaccacaccatt

SEQ ID NO: 40 - DNA, 25 bp, Artificial Sequence
  PCR primers NKG2A F2
ttgatccctgatgctagggtcccag

SEQ ID NO: 41 - DNA, 25 bp, Artificial Sequence
  PCR primers NKG2A R2
ctgacacctgttgttgaattagcga

SEQ ID NO: 42 - DNA, 26 bp, Artificial Sequence
  PCR primers NKG2A R3
tgcatcagaagcgaatactttcaaca

SEQ ID NO: 43 - DNA, 26 bp, Artificial Sequence
  PCR primers NKG2A F3
agggggactactctgttgagtgttca

SEQ ID NO: 44 - DNA, 23 bp, Artificial Sequence
  sgRNA targeting site
ggcacttccagtcattaaagtgg

SEQ ID NO: 45 - DNA, 23 bp, Artificial Sequence
  sgRNA targeting site
aaaagcatgcttacatggtgtgg

SEQ ID NO: 46 - DNA, 23 bp, Artificial Sequence
  sgRNA targeting site
ccttgataggaagttttgaaagg

SEQ ID NO: 47 - DNA, 23 bp, Artificial Sequence
  sgRNA targeting site
taaactaaactgtccttgatagg

SEQ ID NO: 48 - DNA, 23 bp, Artificial Sequence
  sgRNA targeting site
ccagcaatgagtttctctggagg

SEQ ID NO: 49 - DNA, 23 bp, Artificial Sequence
  sgRNA targeting site
gaaactcattgctggtaccctgg

SEQ ID NO: 50 - DNA, 23 bp, Artificial Sequence
  sgRNA targeting site
gtaccagcaatgagtttctctgg

SEQ ID NO: 51 - DNA, 23 bp, Artificial Sequence
  sgRNA targeting site
cctccagagaaactcattgctgg

SEQ ID NO: 52 - DNA, 23 bp, Artificial Sequence
  sgRNA targeting site
tgcttctgcataaagcctgctgg

SEQ ID NO: 53 - DNA, 23 bp, Artificial Sequence
  sgRNA targeting site
gcttctgcataaagcctgctggg

SEQ ID NO: 54 - DNA, 23 bp, Artificial Sequence
  sgRNA targeting site
tgactttcatgactgtcgttagg

SEQ ID NO: 55 - DNA, 23 bp, Artificial Sequence
  sgRNA targeting site
ttcagcttctgtagtacatttgg

SEQ ID NO: 56 - DNA, 23 bp, Artificial Sequence
  sgRNA targeting site
tcagcttctgtagtacatttggg

SEQ ID NO: 57 - DNA, 23 bp, Artificial Sequence
  sgRNA targeting site
tattacttattgactaaaagtgg

SEQ ID NO: 58 - DNA, 23 bp, Artificial Sequence
  sgRNA targeting site
aatctctttcactgatctcatgg

SEQ ID NO: 59 - DNA, 20 bp, Artificial Sequence
  hNKG2A-RT-PCR-F1
ctcgcagctccatttcagtc

SEQ ID NO: 60 - DNA, 21 bp, Artificial Sequence
  hNKG2A-RT-PCR-R1
cagggaagaattgttgtgcct

SEQ ID NO: 61 - DNA, 20 bp, Artificial Sequence
  mNKG2A-RT-PCR-F1
atgcagaactgaaggtggca

SEQ ID NO: 62 - DNA, 20 bp, Artificial Sequence
  mNKG2A-RT-PCR-R1
tctgctgtgagaccagaagc

SEQ ID NO: 63 - DNA, 22 bp, Artificial Sequence
  GAPDH-F
tcaccatcttccaggagcgaga

SEQ ID NO: 64 - DNA, 21 bp, Artificial Sequence
  GAPDH-R
gaaggccatgccagtgagctt

SEQ ID NO: 65 - protein, 179 aa, Rattus norvegicus
MAVSRITRWRLMSMFFGIKCLFLIVALGVLVKNSFTIQNIQSTPSSTPIVEFQKVSKCCACLEKWIGHQCSCYFISKEEKSWKGSREFCASQNSSLLQLQTRNELSFMSSSQAFFWIGIHYNEERSAWLWEDGTFPSKDLFPEFSKFRQDHCIGYSISREISSESCENKNRFICKQLPT

SEQ ID NO: 66 - protein, 236 aa, Rattus norvegicus
MSNDRVTYAELNVAKNSRYQHNKPRGSRSSISVIEQEIVYADFSFKNPSQEQTRICRDCHCKGFPSPPEKLIAGTLGFISFVLIVAVVVITTVATPYTETKAQINSSMTRTYQARPCTRCPDDWISYSHNCYYISVERKSWNDGLTSCISKNCSLLHIDSEEEQAFLQSFSLVSWTGFFRKSRSQPWVWENGSTFKPKITEMLHDEYNCIMMSTSGLTAENCTILHPYVCKCKLTN